(19) United States
(12) Patent Application Publication
(10) Pub. No.: US 2018/0259508 A1
(43) Pub. Date: Sep. 13, 2018

(54) GLYCAN-SPECIFIC ANALYTICAL TOOLS

(71) Applicants: UNIVERSITY OF GEORGIA RESEARCH FOUNDATION, INC., Athens, GA (US); GLYCOSENSORS AND DIAGNOSTICS, LLC, Athens, GA (US)

(72) Inventors: Robert J. Woods, Athens, GA (US); Loretta Yang, San Diego, CA (US)

(21) Appl. No.: 15/916,578

(22) Filed: Mar. 9, 2018

Related U.S. Application Data
(63) Continuation-in-part of application No. 13/148,289, filed on Oct. 28, 2011, now Pat. No. 9,926,612.
Application No. 13/148,289 is a continuation-in-part of application No. 14/001,702, filed on Sep. 18, 2013, now abandoned.
(60) Provisional application No. 61/193,608, filed on Dec. 10, 2008.
(60) Provisional application No. 61/447,925, filed on Mar. 1, 2011.

(51) Int. Cl. G01N 33/53

(52) U.S. Cl. CPC G01N 33/5308; G01N 2400/00

(57) ABSTRACT

Provided are lectenz molecules, which are mutated carbohydrate processing enzyme enzymes that are catalytically inactive and that have had their substrate affinity increased by at least 1.2 fold. Further provided are methods for making and methods of using such lectenz. Further provided are compositions and methods directed to the multiplexed analysis of carbohydrates and carbohydrate containing compounds. The compositions and methods utilize suspension array technology (SAT) and an array of different carbohydrate binding molecules, each carbohydrate binding molecules with a known carbohydrate binding specificity, to obtain a glycoprofile of the carbohydrate structure(s) in a sample. Each carbohydrate binding molecule of a given specificity is linked to the external surface of a population of individually addressable particles.

### CONTINUING APPLICATION DATA

This application is a continuation-in-part of U.S. National Stage application Ser. No. 13/148,289, which is the § 371 U.S. National Stage of International Application No. PCT/US2009/067582, filed 10 Dec. 2009, which claims the benefit of U.S. Provisional Application Ser. No. 61/193,608, filed 10 Dec. 2008, and a continuation-in-part of U.S. National Stage application Ser. No. 14/001,702, which is the § 371 U.S. National Stage of International Application No. PCT/US2012/027211, filed 1 Mar. 2012, which claims the benefit of U.S. Provisional Application Ser. No. 61/447,925, filed 1 Mar. 2011, all of which are incorporated by reference herein.

### GOVERNMENT FUNDING

Part of the work performed during development of this invention utilized U.S. Government funds under R41GM086991 awarded by the National Institutes of Health. Therefore, the U.S. Government has certain rights in this invention.

### BACKGROUND

Unlike protein sequences, which are encoded by the organism's genetic material, the subsequent attachment of complex carbohydrates (glycans) in eukaryotes is controlled by enzymes that either trim or extend the glycan core. A single protein frequently exhibits multiple versions of the glycan, depending on the age or location of the protein. Variations in the glycosylation pattern (glycoprofile) can also result from a range of diseases that introduce mutations into gene sequences, or that alter regulatory control pathways. Aberrant protein glycosylation is therefore a hallmark of several disease states, including diabetes (Coppo and Amore, 2004, Kidney International; 65(5):1544-1547), IgA nephropathy (Amore and Coppo, 2000, Nephron; 86(3):255-259), and various cancers (Krengel et al., 2004, J Biol Chem; 279(7):5597-5603). Because of their exposure on cell surfaces, the glycan chains frequently also serve as receptors for viral and bacterial pathogens (Lim et al., 2008, J Proteome Res; 7(3):1251-63). The ability to characterize glycoprofiles is therefore relevant to disease marker discovery, the development of therapeutics, the study of infectious diseases, and glycobiology research in general. Moreover, the Food and Drug Administration (FDA) requires that the glycoprofiles of all therapeutic glycoproteins fall within accepted limits (Comer et al., 2001, Anal Biochem; 293:169-177). The biologics market is estimated at $100B-$117B annually and is the most rapidly growing sector of the pharmaceutical industry (Abbott et al., 2008, J Proteome Res; 7(4):1470-80; Kaneda et al., 2002, J Biol Chem; 277(19):16928-16935).

Currently, rapid and affordable tools for determining or monitoring protein glycosylation patterns do not exist. Instead, glycoprofiling typically employs techniques, such as mass spectrometry (MS) (Bechtel et al., 1990, J Biol Chem; 265(4):2028-2037), which are dependent on costly instrumentation and highly trained personnel. These technologies are also poorly suited for real-time monitoring of glycoprofiles, as for example during the expression of therapeutic proteins. There is a need for rapid, simple, reliable, and affordable tools for determining or monitoring protein glycosylation patterns.

The present invention is directed to glycan-specific analytical tools, their methods of use, and processes for making glycan-specific analytical tools. Other analytical tools are further provided herein.

Glycans are complex carbohydrates commonly found attached to lipids and proteins. Because of their presence on protein and cell surfaces, complex carbohydrates often occupy a functional position in biological recognition processes. The complex shape, functionality, and dynamic properties of oligo- and polysaccharides allow these molecules to function in intermolecular interactions as encoders of biological information.

Carbohydrate recognition is an integral part of normal biological development, but can also be used by the innate immune system to allow a host organism to identify a foreign pathogen, on the basis of the carbohydrates presented on the surface of the pathogen. Conversely, many bacterial and viral pathogens initially adhere to host tissues by binding specifically to carbohydrates on the host's cell surfaces. Thus, there is an interest in developing therapeutic agents that can interfere with carbohydrate-based host-pathogen interactions or that can function as antibacterial vaccines. Abnormal glycosylation is also a marker for certain types of cancer and other diseases, making them targets for diagnostic and therapeutic applications. For example, the state of modification of intracellular proteins by O-linked N-acetylglucosamine (O-GlcNAcylation) is an important biomarker of changes caused by disease, notably type-2 diabetes mellitus.

Despite the importance of glycans in biological development and disease, there is at present a lack of sufficient glycan-specific analytical tools, which has delayed exploiting aberrant glycosylation in the diagnosis and treatment of disease. For example, a current method for monitoring O-GlcNAc incorporation in cells, and subsequent presentation on proteins, is based on exogenous uptake of labeling reagents, such as N-azidoacetylglucosamine (GlcNAz). Unfortunately, this method is not applicable to the analysis of O-GlcNAc in isolated tissue or protein samples. An alternative O-GlcNAc labeling approach that can be applied in glycomic/proteomic analyses uses chemoenzymatic tagging. A serious limitation of this method is that it also labels other GlcNAc-terminated complex glycans. Thus, there remains a need for analytical tools with defined carbohydrate specificity that can be used to interrogate biological samples in the search for abnormal glycosylation.

Currently, two major types of biomolecules used in glycan-specific analytical applications are sugar-binding proteins (lectins) and antibodies. A major drawback associated with either of these types of reagents is the characteristically weak interactions between carbohydrates and proteins, with dissociation constants typically in the range of milli- to micromolar for lectins and micro- to nanomolar for antibodies. Additionally, a significant difficulty in using antibodies is that carbohydrates are very poor immunogens. They are generally unable to generate a T-cell dependent response and so produce most often IgM class antibodies, which are inconvenient for analytical and diagnostic applications. Single chain chimeras consisting of the variable domains of the heavy and light chains (scFv) can suffer from instability. Additionally, glycan-specific analytical techniques employing antibodies suffer a drawback due to the selectivity of antibodies being context dependent. Alternatively, lectins, with their broad specificity, are limited in their use for analytical applications. Therefore, there exists a need for developing analytical reagents that possess sufficient specificities to the carbohydrate sequence, yet are able to recognize the sequence within a broad range of glycans.

### SUMMARY OF THE INVENTION

The present invention provides a lectenz comprising a carbohydrate-processing enzyme that has been mutated to eliminate its catalytic activity while maintaining its substrate specificity. In certain embodiments, the lectenz of the present invention has an affinity to glycans that is higher than the Kₘ of the wild-type enzyme. The lectenz of the present invention may also have a markedly decreased k_{off} rate. In other embodiments, the present invention presents a lectenz comprising a catalytically inactive carbohydrate-processing enzyme, wherein the inactive enzyme comprises one or more amino acid residues that differ from the wild-type residues, said residues are selected from a list consisting of the residues that are proximal to the bound substrate, but which contribute less than about |0.5 kcal/mol| to a gas-phase (ΔE_{MM}) interaction energy, that contribute less than about |0.5 kcal/mol| to a total (ΔG) interaction energy, or any residues that contribute unfavorably to the binding interaction energy, and combinations thereof Residues that are proximal to the substrate are generally considered here to be within 5 Å of the substrate, but could be farther or closer.

In another aspect, the present invention provides a method for generating a lectenz. In some embodiments, the method comprises: (a) analyzing a sequence of a carbohydrate-processing enzyme for one or more amino acid residues that, when mutated, could affect the affinity of the carbohydrate-processing enzyme to a glycan or the stability of a enzyme-glycan complex; (b) performing a computational simulation to predict binding energies of the enzyme-glycan complex, wherein the carbohydrate-processing enzyme has at least one mutated amino acid identified in step (a); (c) testing carbohydrate-processing enzymes comprising mutations identified in steps (a) and (b) for their ability to form the complex; and (d) identifying mutants from step (c) that exhibit binding affinities to the glycan that are greater than those of WT enzyme.

Another aspect of the present invention provides methods of using lectenz for glycan-specific analytical applications. In certain embodiments, lectenz of the present invention can be used as affinity reagents or as vehicles for tissue staining. In other embodiments, lectenz can be used for enriching a biological sample with a particular glycoform. In yet other embodiments, lectenz find their application for determining specific glycosylation sites on glycoproteins. Other aspects of the present invention involve use of lectenz as vehicles for targeted delivery of active therapeutic agents.

In certain embodiments, the carbohydrate-processing enzyme is a glycosidase enzyme. In other embodiments, the carbohydrate-processing enzyme is a glycosyltransferase enzyme. In other embodiments, the carbohydrate-processing enzyme is a polysaccharide lyase enzyme. In other embodiments, the carbohydrate-processing enzyme is a sulfatase enzyme. In other embodiments, the carbohydrate-processing enzyme is a sulfotransferase enzyme. In other embodiments, the carbohydrate-processing enzyme is a ligase enzyme. In further embodiments, the carbohydrate-processing enzyme is an amidase enzyme. In yet further embodiments, the carbohydrate-processing enzyme is an epimerase enzyme.

The present invention includes a composition having a plurality of individually addressable particles, each individually addressable particle having an external surface and having linked to said external surface a separate carbohydrate binding molecule.

In some embodiments of the composition, the carbohydrate binding molecules are independently selected from the group consisting of lectins, antibodies, LECTENZ molecules (carbohydrate processing enzymes that have been inactivated but still bind to carbohydrate(s) with high specificity), carbohydrate-binding proteins, carbohydrate binding domains of proteins, pathogen adhesion domains, and aptamers. In some embodiments, the LECTENZ molecule is derived from an enzyme selected from the group consisting of a glycosidase enzyme, a glycosyltransferase enzyme, polysaccharide lyase enzyme, sulfatase enzyme, a sulfotransferase enzyme, a ligase enzyme, an amidase enzyme, and an epimerase enzyme. In some embodiments, the LECTENZ molecule is derived from PNGaseF or O-GlcNAcase.

In some embodiments of the composition, individually addressable particles include beads or nanoparticles.

In some embodiments of the composition, each individually addressable particle is separately labeled with a detectable label. In some embodiments, the detectable label is an optically encoded fluorescent dye.

In some embodiments, the composition is formulated for flow cytometry analysis.

In some embodiments, the composition is formulated for image based analysis.

In some embodiments, the composition is formulated for research, industrial, medical, or veterinary use.

The present invention includes kits including a composition as described herein, packaging materials and instructions for use.

The present invention includes kits having one or more compositions, each composition having individually addressable particles; each individually addressable particle having an external surface and having linked to said external surface a separate carbohydrate binding molecule; and each individually addressable particle separately labeled with a detectable label.

In some embodiments, a kit further includes a secondary detection reagent for detectably labeling an analyte.

In some embodiments, a kit further includes positive and/or negative analyte controls.

In some embodiments, a kit further includes instructions for use.

In some embodiments, a kit is formulated for research, industrial, medical, or veterinary use.

In some embodiments, a kit is formulated for flow cytometry analysis.

In some embodiments, a kit is formulated for image based analysis.

In some embodiments, a kit further includes a software component to assist in the calculation of relative glycan proportions in a sample.

The present invention includes a multiplex detection method for detecting a carbohydrate or a carbohydrate containing compound in a sample, the method including contacting the sample with a solution having a plurality of individually addressable particles, each individually addressable particle having an external surface and having linked to said external surface a separate carbohydrate binding molecule; and detecting the binding of the carbohydrate or carbohydrate containing compound to one more individually addressable particles; wherein the carbohydrate or carbohydrate containing compound bound to one more individually addressable particles remains in suspension.

In some embodiments of the method, detecting a carbohydrate or carbohydrate containing compound includes detecting the structure of the carbohydrate.

In some embodiments of the method, each separate carbohydrate binding molecules is independently selected from the group consisting of lectins, antibodies, LECTENZ molecules (carbohydrate processing enzymes that have been inactivated but still bind to carbohydrate(s) with high specificity), carbohydrate-binding proteins, carbohydrate binding domains of proteins, pathogen adhesion domains (such as cholera toxin B, other toxins, and hemagglutinin), aptamers including protein, RNA or other small molecule aptamers, and any other molecule that naturally binds or is engineered to bind a carbohydrate.

In some embodiments of the method, the individually addressable particles include beads and/or nanoparticles.

In some embodiments of the method, each individually addressable particle is separately labeled with a detectable label. In some embodiments, the detectable label is an optically encoded fluorescent dye.

In some embodiments of the method, detection is by flow cytometry analysis.

In some embodiments of the method, detection is by image based analysis.

In some embodiments of the method, at least one of the detected carbohydrates or carbohydrate containing compounds is detectable labeled. In some embodiments, the method further includes co-detecting the detectably labeled individually addressable particle and the detectably labeled carbohydrates or carbohydrate containing compounds.

In some embodiments of the method, the carbohydrate includes at least one monosaccharide.

In some embodiments of the method, the carbohydrate includes a polymer including at least two monosaccharides, and wherein detecting the structure of the carbohydrate includes detecting at least one feature selected from the group consisting of constituent monomer, functional group, linkage position, linkage stereochemistry, presence or absence of branching, branch position.

In some embodiments of the method, the carbohydrate or carbohydrate containing compound is selected from the group consisting of a monosacharide, disaccharide, trisaccharide, oligosaccharide, polysaccharide, glycoside, glycan, glycosaminoglycan, glycoprotein, glycopeptide, glycolipid, glycolipopeptide, nucleotide, nucleoside, nucleoside phosphate, and nucleic acid.

In some embodiments of the method, the sample is obtained during the production of a recombinant glycoprotein in the pharmaceutical or research industries.

In some embodiments of the method, glycosylation profiles are monitored during bioprocessing.

In some embodiments, the sample includes at least one chemically or enzymatically synthesized carbohydrate or carbohydrate containing compound.

In some embodiments, a sample is an environmental or biological sample.

In some embodiments, a sample is or is from a microorganism. In some embodiments, the microorganism is a virus, bacterium, yeast, fungus or protozoan.

In some embodiments, the sample is from a plant or an animal. In some embodiments, the animal is a mammal. In some embodiments, the mammal is a human.

The present invention includes software that the converts one or more intensities measured in a method described herein into a percentage of glycan present in the sample.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

The term “and/or” means one or all of the listed elements or a combination of any two or more of the listed elements.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

The terms “comprises” and variations thereof do not have a limiting meaning where these terms appear in the description and claims.

Unless otherwise specified, “a,” “an,” “the,” and “at least one” are used interchangeably and mean one or more than one.

Also herein, the recitations of numerical ranges by endpoints include all numbers subsumed within that range (e.g., 1 to 5 includes 1, 1.5, 2, 2.75, 3, 3.80, 4, 5, etc.).

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

### BRIEF DESCRIPTION OF THE DRAWINGS

FIG. 1 depicts the relationship between a carbohydrate processing enzyme (a neuraminidase in the example) and its carbohydrate binding lectenz analog.

FIG. 2 depicts a protocol combining computational and in vitro display library methods to optimize the affinities of lectenz.

FIG. 3 depicts the RMSD in the Cα positions in the PNGase F complex.

FIG. 4 depicts the hydrogen-bond interaction scheme for the binding of chitobiose to PNGase F.

FIG. 5 depicts, in the left image: residues within 4.5 Å of the disaccharide ligand (dark grey) in the binding site of PNGase F. In the right image: the solvent accessible surface with the residues identified as most significant for binding labeled.

FIG. 6 depicts SPR sensograms indicating the variations in kinetic on- and off-rates as a function of mutation and temperature (10° C. and 25° C.).

FIG. 7 depicts a protein display library fused to the Aga2 protein in yeast. Detection of a fluorescently labeled antigen binding to c-myc tagged protein is illustrated.

FIG. 8 depicts a cell sorting via flow cytometry indicating the selection of high affinity clones.

FIG. 9 depicts the enhanced affinity of a preliminary lectenz relative to the inactive enzyme.

FIG. 10 shows a schematic representation of the multiplexed interactions between multiple suspension array technology (SAT) reagents and a glycoprotein analyte. Glycan specific lectins are conjugated to red fluorescent multiplex microspheres (beads), and then incubated with a green fluorescently labeled glycoprotein. The amount of glycoprotein bound to each bead is measured using flow cytometry.

FIG. 11 shows how in flow cytometry particles in a sample are hydrodynamically focused and flow in a single file through a detector, as light scatter and fluorescence emission are measured for each particle.

FIG. 12 shows a conceptual representation of real-time monitoring of glycosylation during protein expression.

FIG. 13 shows a representative scatter dot plot of Multiplexed Suspension Glycoprofiling Array beads (left) and GlcNAcβ1-4GlcNAcβ-PAA-fluorescein bound (right). Bead 1-ethanolamine quenched; Bead 2-SNA I; Bead 3-MAL II; Bead 4-GS II; Bead 5-ConA; and Bead 6-ECA.

FIG. 14 shows specific detection of directly-labeled GlcNAcβ1-4GlcNAcβ-PAA-fluorescein by MSA element GSII, which is specific for terminal GlcNAc. Intensities for beads with no reagent were subtracted.

FIG. 15 shows secondary detection of GlcNAcβ1-4GlcNAcβ-PAA-biotin by MSA element GSII, which is specific for terminal GlcNAc. Intensities for beads with no reagent were subtracted.

FIG. 16 shows secondary detection of Neu5Acα2-6[Galβ1-4GlcNAcβ1-3]2β-Sp-Biotin by MSA element SNA I, which is specific for the terminal Neu5Acα2-6Gal sequence. Intensities for unlabeled (blank) beads were subtracted.

FIGS. 17A and 17B show binding of GM1 (GM1-LC-LC-biotin). Intensities for beads with no reagents were subtracted.

FIGS. 18A and 18B show binding of biotinylated fetuin and asialofetuin glycoproteins. FIG. 18A shows binding of fluorescently labeled fetuin and asialofetuin glycoproteins, average of three experiments. FIG. 18B shown the difference in binding between fetuin and asialofetuin. Intensities for beads with no reagents were subtracted.

It is understood that the illustrations and figures of the present application are not necessarily drawn to scale and that these figures and illustrations merely illustrate, but do not limit, the present invention.

### DETAILED DESCRIPTION OF THE INVENTION

In the following description, for purposes of explanation, specific numbers, parameters and reagents are set forth in order to provide a thorough understanding of the invention. It will be apparent, however, that the invention may be practiced without these specific details. In some instances, well-known features may be omitted or simplified so as not to obscure the present invention.

The embodiment(s) described, and references in the specification to “one embodiment”, “an embodiment”, “an example embodiment”, etc., indicate that the embodiment(s) described can include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is understood that it is within the knowledge of one skilled in the art to effect such feature, structure, or characteristic in connection with other embodiments whether or not explicitly described.

### Definitions

As used herein, a carbohydrate-processing enzyme is a term used to refer to any enzyme that acts on a carbohydrate-containing substrate. Examples include glycosidases, glycosyltransferases, but are not limited to those.

As used herein, the term “glycosidase” is used to refer to an enzyme that catalyzes a hydrolysis of a glycosidic bond. The term “glycosidic bond” refers to a type of a functional group that joins a carbohydrate molecule to another carbohydrate molecule or that joins a carbohydrate molecule to a protein molecule or that joins a carbohydrate molecule to a lipid molecule. The term “carbohydrate” is meant to refer to an organic compound of a general formula Cₘ(H₂O)ₙ. For the purposes of the present invention, terms “carbohydrate”, “complex carbohydrate”, and “glycan” are used interchangeably.

The terms “catalytically inactive mutant” or “mutant” or “inactive enzyme” are used interchangeably, and refer to an enzyme that has lost at least 95% of its catalytic activity, and that has an amino acid composition different than the catalytically active enzyme. Stated otherwise, a rate of chemical bond cleavage by the catalytically inactive mutant is, at the most, 5% greater than the rate of the bond cleavage measured under the identical conditions in the absence of any catalyst. By “catalytically active enzyme” it is meant to refer to a protein capable of catalyzing a hydrolysis of a chemical bond. The term “wild-type (WT) enzyme” refers to an enzyme encoded by a gene that has a sequence of a gene as it naturally occurs in an organism, and that has not been altered by human intervention. It is of course understood that a naturally occurring polymorphic form of wild-type enzyme is included within this definition. It is further understood that modifications such as tags or other modifications used in the purification or isolation of a protein that do not otherwise change the natural start or stop codon of a protein fall within the definition of a WT enzyme for purposes of this invention. As used herein, the term “ligand” and “substrate” are used interchangeably, and refer to a molecule to which WT or mutant enzymes can bind.

The lectenz of the present invention have an affinity for the glycan that is higher than the Kₘ of the wild-type enzyme. To understand the meaning of Kₘ, you need to have a model of enzyme action. The simplest model is the classic model of Michaelis and Menten, which has proven useful with many kinds of enzymes (Equation 1).

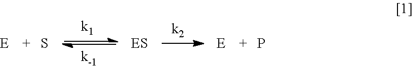

The substrate (S) binds reversibly to the enzyme (E) in the first reaction. In most cases, you can not measure this step. What you measure is production of product (P), created by the second reaction. The Michaelis and Menten constant (Km) is defined in Equation 2.

$${K}_{m} = \frac{{k}_{2} - {k}_{- 1}}{{k}_{1}} \qquad [ 2 ]$$

Note that Km is not a binding constant that measures the strength of binding between the enzyme and substrate. Its value includes the affinity of substrate for enzyme, but also the rate at which the substrate bound to the enzyme is converted to product. Only if k2 is much smaller than k−1 will KM equal a binding affinity. It is understood that in the context of a wild-type enzyme that it is difficult to directly measure affinity values, because the WT enzyme is acting on the ligand, for this reason it is convenient to compare the K_{d} of the inactive enzyme to the Kₘ of the wild-type enzyme. As used herein, the term “affinity” means a force of attraction between two molecules. Although normally measured relative to the Kₘ of the WT enzyme, the affinity of the lectenz can also be expressed in terms of a decrease in the dissociation constant, K_{d} for its ligand relative to an inactive form of the WT enzyme. The dissociation constant, K_{d}, is an equilibrium constant that measures the propensity of a complex to dissociate into its constituents. For a general reaction:

iE⇄E+S [3]

Wherein, iE and S designate inactive enzyme and substrate, respectively, and iES is the inactive-enzyme-substrate complex. The corresponding dissociation constant, K_{d}, is then defined as:

$${K}_{d} = \frac{[ iE ] [ S ]}{[ iES ]} \qquad [ 4 ]$$

wherein [iE] and [S] designate concentrations of free inactive-enzyme and substrate, respectively, and [iES] is a concentration of the inactive-enzyme-substrate complex. Therefore, in certain embodiments, the increase in affinity of the lectenz is measured by comparing the affinity of the lectenz to a catalytically-inactive form of the WT enzyme that has not been subjected to additional affinity-optimizing mutations. For the purposes of the present invention, the affinity of a protein for its ligand can be expressed in dissociation (K_{d}) or association (Kₐ) constants.

The expression “ΔG” is referred to the Gibbs free energy of binding. The Gibbs free energy is a thermodynamic potential that represents the work which must be done in acting against the forces which hold a complex together, while disassembling the complex into component parts separated by sufficient distance that further separation requires negligible additional work. The expression “ΔE_{MM}” refers to molecular mechanics free energy in gas-phase. Terms “specificity” or “enzyme specificity” are used interchangeably, and refer to an ability of an enzyme to recognize and select ligands containing specific molecular structures from a population of different ligands. The term “non-specific” binding or interaction refers to an event of weak interactions between molecules or residues that is not based on any specific recognition or discrimination of individual molecules or residues.

The term “Molecular Dynamics (MD)” is meant to refer to a form of computer simulation in which atoms and molecules are allowed to interact for a period of time by approximations of known physics, giving a view of the motion of the particles. Classical MD simulations are governed by Newton's equations of motion employing energies and forces derived from a classical force field. A classical force field is a mathematic model that relates the atomic positions in a molecule or aggregate of molecules to the potential energy of the molecule or aggregate. The terms “Ewald treatment” or “Ewald summation”, as used herein, describes a method for computing the interaction energies of periodic systems (e.g. crystals), particularly electrostatic energies. By the terms “Verlet algorithm” or “Verlet integration”, it is meant a numerical method used to integrate Newton's equations of motion.

### Lectenz

One aspect of the present invention provides a lectenz. A lectenz of the present invention comprises a catalytically inactive mutant of a carbohydrate-processing enzyme that has substantially the same specificity for a given glycan as the wild-type enzyme, and an increased affinity towards the glycan as compared to the WT enzyme. As used herein, the term “substantially the same” is meant to describe a specificity of the glycosidase mutant that is at least 60% of the wild-type enzyme. In some embodiments, the specificity of the mutant is at least 70% of the WT enzyme. In at least one embodiment, the mutated glycosidase is at least 85% as specific to its substrate as the wild-type enzyme to the same substrate. In other embodiments, the mutated glycosidase is at least 95% as specific to its substrate as the wild-type enzyme to the same substrate.

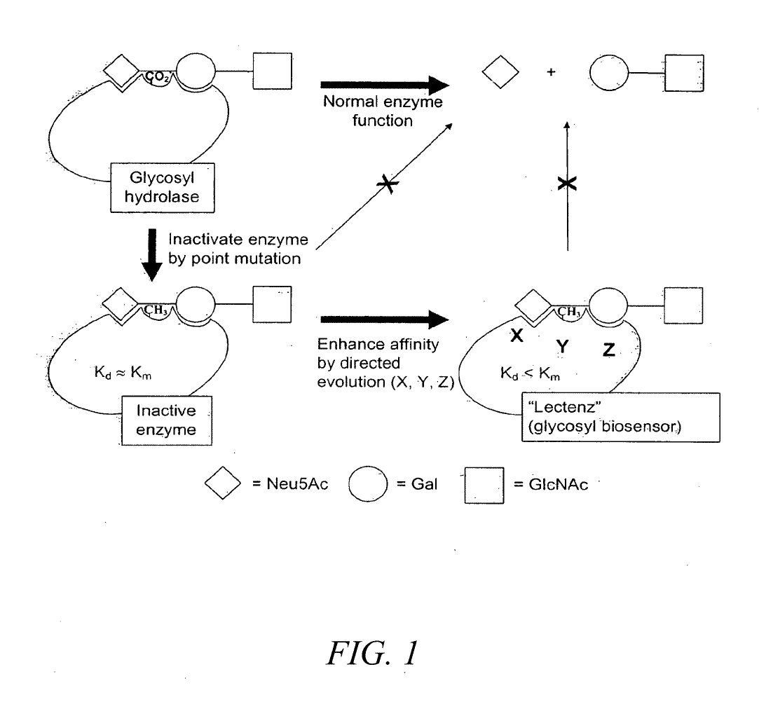

For an engineered lectenz the mutation of the active site residues offers a route not only to inactivating the enzyme, but potentially to enhancing affinity (FIG. 1). In some embodiments, the affinity of the lectenz towards the glycan is increased from that of the wild-type enzyme. In certain embodiments, the affinity of an lectenz to its substrate can be expressed in terms of a dissociation constant, K_{d}, (See Equations 3 and 4). The smaller the dissociation constant, the more tightly the lectenz is bound to the substrate. In some embodiments of the present invention, the dissociation constant (K_{d}) of the lectenz towards the glycan is at least about 1.2 to about 1,000-fold less than the Kₘ of the WT carbohydrate-processing enzyme. In other embodiments, the dissociation constant of the lectenz towards the glycan is at least about 2 fold less than the Kₘ the WT carbohydrate-processing enzyme. In certain embodiments, the dissociation constant of the lectenz towards the glycan is at least about 10 fold less than the Kₘ of the WT carbohydrate-processing enzyme. In certain embodiments the dissociation constant of the lectenz towards the glycan is at least about 10,000 fold less than the Kₘ of the WT carbohydrate-processing enzyme. In further embodiments the dissociation constant of the lectenz towards the glycan is at least about 100,000 fold less than the Kₘ of the WT carbohydrate-processing enzyme.

It is also understood that the affinity improvement of the lectenz of the present invention can be expressed in terms of a decrease in K_{d} relative to that of an inactive mutant of the WT enzyme. Thus, the K_{d} of the lectenz towards the glycan is at least about 1.2 to about 1,000-fold less than that of the inactive WT carbohydrate-processing enzyme. In other embodiments, the dissociation constant of the lectenz towards the glycan is at least about 2 fold less than the K_{d} of the WT carbohydrate-processing enzyme. In certain embodiments, the dissociation constant of the lectenz towards the glycan is at least about 10 fold less than the K_{d} of the WT carbohydrate-processing enzyme. In certain embodiments the dissociation constant of the lectenz towards the glycan is at least about 10,000 fold less than the K_{d} of the WT carbohydrate-processing enzyme. In further embodiments the dissociation constant of the lectenz towards the glycan is at least about 100,000 fold less than the K_{d} of the WT carbohydrate-processing enzyme.

Lectenz of the present invention are not limited to any specific carbohydrate-processing enzyme. Rather, the present invention is broadly applicable to any glycosidase or glycosyltrasferase enzyme, protein, or polypeptide capable of specifically recognizing a carbohydrate. Examples of glycosidases suitable for the present inventions include, but are not limited to, lactase, amylase, chitinase, sucrase, maltase, neuraminidase, invertase, hyaluronidase, and lysozyme. It is understood that glycosidases categorized by the Enzyme Commission (EC) number 3.2.-.-, wherein “-” is a number, are included in the present invention. Glycosidases of the present invention can be inverting or retaining glycosidases. In one embodiment, the lectenz of the present invention is prepared from PNGase F, isolated from Flavobacterium meningosepticum. In another embodiment, the lectenz is prepared from recombinant β-O-GlcNAcase, with the WT sequence as determined for β-O-GlcNAcase isolated from Bacteroides thetaiotaomicron. In yet another embodiment, neuraminidase from Clostridium perfringens is used to prepare the lectenz. In addition to glycosidases, carbohydrate-processing enzymes suitable for use in the present invention include glycosyltransfeases, including those designated under EC number 2.4.-.-, and polysacharide lyases, including those designated under EC number 4.2.2.-. Other carbohydrate-processing enzymes include carbohydrate esterases, sulfatases, sulfotransferases, or any other enzyme that acts on a carbohydrate substrate. Catalytically inactive carbohydrate-processing enzymes of the present invention can be prepared from carbohydrate-processing enzymes isolated from prokaryotic or eukaryotic organisms, as well as others.

In certain embodiments, the lectenz of the present invention will have high thermal stability. By high thermal stability it is meant that the lectenz retains its tertiary structure at a temperature of at least 40° C. for at least thirty minutes in a physiologically acceptable buffer. A physiologically acceptable buffer generally refers to a 0.01-0.2 M phosphate buffer of pH 6-8, 0-1% NaCl concentration, and 0-20 mM glucose concentration. In certain embodiments, the lectenz remains properly folded at a temperature of at least 60° C. for at least thirty minutes in a physiologically acceptable buffer. In other embodiments, the lectenz retains its tertiary structure at a temperature of at least 80° C. for at least thirty minutes in a physiologically acceptable buffer. If needed, lectenz of the present invention can be prepared from glycosidases isolated from thermophilic or hyperthermophilic organisms. Examples of thermophilic and hyperthermophilic organisms from which carbohydrate-processing enzymes suitable herein can be isolated include, but are not limited to, Thermus thermophilus, Spirochaeta americana, Pyrococcus furiosus, Methanopyrus kandleri, Pyrolobus fumarii, Geothermobacterium ferrireducens, and Archaeoglobus fulgidus.

In some embodiments, a lectenz of the present invention is characterized by long kinetic off-rates. Kinetic off-rate is measured by a dissociation rate constant (k_{off}), or a speed with with a ligand will dissociate from a protein (see Equation 5). Generally, substrate-inactive-enzyme complex formation can be described by the following kinetic mechanism:

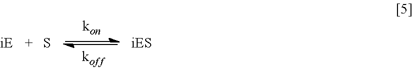

wherein iE and S designate inactive-enzyme and substrate, respectively, iES is the inactive-enzyme-substrate complex, and kₒₙ and k_{off} are rate constants for complex formation and dissociation, respectively. In at least some embodiments, a lectenz of the present invention will dissociate from its ligand at a rate that is at least about 2-fold slower than that of the WT enzyme. In one embodiment, a rate of a lectenz-glycan complex dissociation is at least 5-fold slower than that of a WT-glycan complex. In yet another embodiment, the rate of dissociation of the lectenz-glycan complex is at least 10-fold slower than that of WT. Comparisons can also be made between the off rate of the lectenz and that of an inactivated WT enzyme. In at least some embodiments, a lectenz of the present invention will dissociate from its ligand at a rate that is at least about 2-fold slower than that of the inactive WT enzyme. In one embodiment, a rate of a lectenz-glycan complex dissociation is at least 5-fold slower than that of a complex between an inactive WT enzyme and a glycan. In yet another embodiment, the rate of dissociation of the lectenz-glycan complex is at least 10-fold slower than that of inactive WT enzyme.

In certain embodiments, the catalytically inactive mutant of the present invention has one or more amino acid residues that differ from the WT residues, and that are selected from residues that are proximal to the substrate in the enzyme-substrate complex, but that contribute more than about −0.5 kcal/mol to total (ΔG) interaction energy, or any residues that contribute unfavorably to the binding interaction energy, and combinations thereof. This set of residues that is close to the substrate, but not forming strong interactions may be termed a tepid set of residues. In certain embodiments, the mutant glycosidase has two or more mutations in the tepid residue set. In other embodiments, the mutant glycosidase has three or more, four or more, or even five or more mutations at these positions.

### Computer-Aided Methods for Generating a Lectenz

Another aspect of the present invention provides a computer-aided method for generating a lectenz, wherein the lectenz comprises a catalytically inactive mutant of a carbohydrate-processing enzyme, the method comprising:
(a) analyzing a sequence of a carbohydrate-processing enzyme for one or more amino acid residues that, when mutated, could inactivate the enzyme;
(b) performing a computational simulation to predict binding energies of the WT enzyme-glycan complex, or of a complex wherein the carbohydrate-processing enzyme has at least one mutated amino acid identified in step (a);
(c) subdividing the residues on the basis of their predicted interaction energies into two groups, namely, a first group of residues that are essential to defining the specificity of the enzyme, and a second group of residues that are proximal to the substrate but not found to be essential to defining specificity (this second set is referred to herein as tepid residues);
(d) testing carbohydrate-processing enzymes comprising mutations identified in steps (a), (b) and (c) for their ability to form the enzyme-glycan complex; and
(e) identifying mutants from step (d) that exhibit binding affinities to the glycan that are at least 1.2-fold greater than those of WT glycosidase.

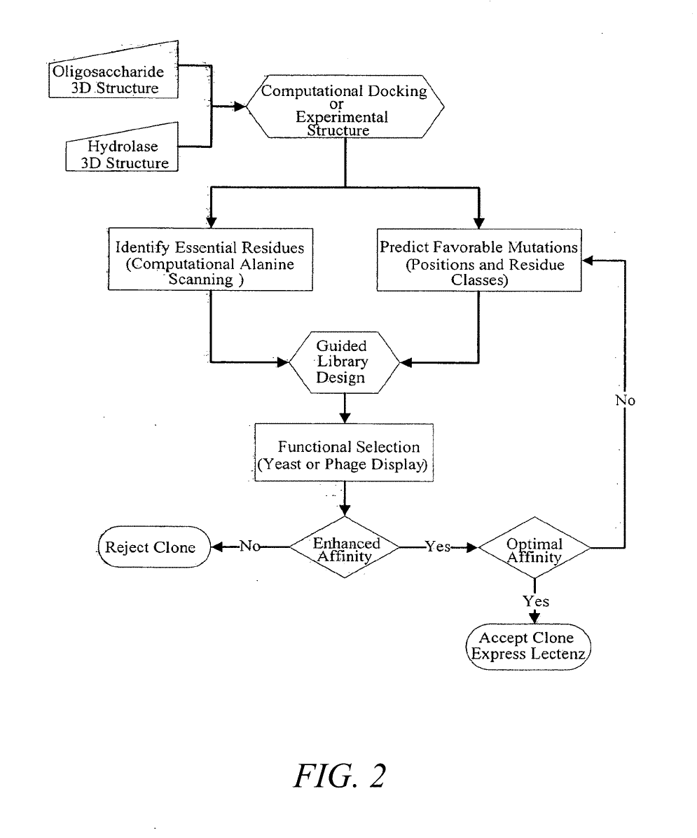

This embodiment, which is outlined in material form in FIG. 2, is understood to represent only a single embodiment for arriving at the lectenz of the present invention. The steps of FIG. 2, which are described in greater detail herein, can be modified as explained herein to arrive at the lectenz of the present invention. Specifically, for example, to the extent that a particular step in FIG. 2 makes reference to a particular technique, such as “Computational Alanine Scanning” for the “Identify Essential Residues” step, as explained in greater detail elsewhere herein, that is a mere embodiment of the invention and there are other approaches for performing the same step.

Certain embodiments of the present invention involve the identification of carbohydrate-processing enzymes suitable for conversion into a lectenz. While production of a lectenz is not limited to a specific carbohydrate-processing enzyme, it might be preferable to select a carbohydrate-processing enzyme(s) wherein its active site residues are known, and/or for which catalytically inactive mutants have been described. Identification of the catalytic residues has been performed for many carbohydrate-processing enzymes using site-directed mutagenesis and confirmed in many cases by measurements of enzyme kinetics. However, the inactive enzymes (first-generation lectenz) have affinities that are often comparable to lectins.

Generally, before conducting computer-aided mutagenesis techniques, it is preferable to predict the binding affinities of a catalytically inactive carbohydrate-processing enzyme for a ligand of interest. There are a number of well known techniques for characterizing the affinity of a carbohydrate-processing enzyme to its ligand. One such technique is a molecular dynamics (MD) technique that employs the AMBER/GLYCAM protein/carbohydrate force field.

Prior to energy analysis, the root mean squared difference (RMSD) in the positions of the Cα atoms can be determined as a function of the simulation time to determine the stability of the MD simulation and the level of conformational equilibration. On the basis of such data, it can be determined whether the average RMSD was stable and within a range of about 0 Å to 4 Å. It is also possible to discern the time required to reach conformational equilibrium through this approach. It is generally preferable to omit the non-equilibrated portion of the data set in regards to subsequent analyses. In one embodiment, where 5 nanoseconds of data were collected and the system took about a nanosecond to equilibrate, the first nanosecond of data was omitted. It is understood that the 5/1 nanosecond embodiment described above is merely exemplary and is not limiting of the present invention.

Ligand stability in the binding site can be assessed by evaluating intermolecular hydrogen bonds between the glycan and the carbohydrate-processing enzyme. Average values for the hydrogen bonds and their percentage occupancies can be collected, and if possible they are collected along with the crystallographically determined values. By monitoring the RMSD of the position of the ligand in the binding site it is also possible to determine ligand stability in the binding site. Having confirmed that the MD simulation is stable and able to reproduce the experimental interactions between the ligand and the protein, one can then employ that system in subsequent analyses. Typical simulations can be performed under constant pressure and temperature (NPT) conditions or under constant volume and temperature (NVT) conditions. These simulations can be performed with the SANDER module of AMBER and the TIP3P water model. In certain embodiments, protein force field parameters are taken from the Parm99 set and carbohydrate parameters from GLYCAM06. It is also possible to perform the simulations with implicit solvent models under non-periodic boundary conditions.

AMBER is a molecular modeling and simulation package that provides simulation-based methods for structure-based ligand design and understanding of structure and free energy in any complex molecular system. AMBER was developed at and is available from University of California, San Francisco. Other modules within AMBER can be employed to perform these MD simulations. Indeed, programs other than AMBER exist for performing MD simulations. Such programs are also applicable to the present invention.

Under certain embodiments, initial coordinates for the glycan-protein complexes can be selected from crystallographic data from inactive enzyme-substrate, active enzyme-inhibitor, or enzyme-product complexes, if available. A theoretical model for the protein can also be employed, such as a model generated by homology or comparative modeling. When only a structure of the free enzyme is available, a co-complex can be predicted using AutoDock or another equivalent program. AutoDock is a suite of automated docking tools designed to predict how small molecules bind to a receptor of known 3D structure. Other docking programs exist and would be applicable to the present invention.

In certain embodiments, histidine protonation states can be inferred from intramolecular hydrogen bonds where possible, otherwise the histidine can be treated as neutral, protonated at N. epsilon. Any net charge on the complex can be neutralized by the addition of the appropriate number of counter ions (Cl⁻ or Na⁺). Typically, the oligosaccharide-protein complexes will be solvated by, for example, ˜10,000 TIP3P water molecules, in a periodic cube with a minimum distance between the edge of the box and the closest atom of the solute of 10 Å. Periodic boundary conditions can be applied together with Ewald treatment of long-range electrostatics with a direct space cutoff distance of 12 Å. It is understood that these parameters are not limiting of the invention. Indeed, it is understood that TIP3P is but just one of the classical water models used for computational chemistry. Other water models, such as TIP4P, TIP5P, SPC, BNS, and others, can be used in the present invention. The water can also be approximated using implicit solvation models such as a dielectric constant, a distance-dependent dielectric constant, a generalized Born model, or by the Poisson-Boltzmann approximation.

In certain embodiments, the initial configurations can be energy minimized with the SANDER module. In one embodiment, the initial configurations comprise 5,000 cycles of steepest descent and 25,000 cycles of conjugate gradient energy minimization with the SANDER module. The entire system can then be subjected to simulated annealing by heating followed by cooling. In certain embodiments, the simulated annealing comprises from 5 to 300K in 50 ps, followed by cooling to 5K in another 50 ps. Initial atomic velocities can be assigned from a Boltzmann distribution, generally at 5K. Prior to the production dynamics stage, the entire system can be thermally equilibrated by heating again from 5 to 300K in 150 ps. A 2 fs time step can be used to integrate the equations of motion, using the Verlet algorithm. Bonds containing hydrogen can be constrained to their equilibrium lengths using the SHAKE algorithm. It is understood that these parameters are exemplary only and are not limiting of the invention.

The method of the present invention is not limited to any particular ligand. Ligands suitable for present invention include any natural or synthetic carbohydrate or derivative thereof. Examples of suitable ligands include, but are not limited to, lactose, sucrose, maltose, trehalose, cellobiose, chitobiose, N-linked oligosaccharides, O-linked oligosaccharides, oligosaccharides, monosaccharides, terminal branched and non-branched α-(2,3) and α-(2,6)-Neu5Ac, α-(1-2)-man on high mannose N-glycans, α-Gal on glycoproteins and glycolipids, glycosaminoglycans (such as heparin, heparan, chondroitin, hyaluronic acid and their sulfated analogs), β-N- and β- or α-O-GlcNAc on glycoproteins and glycolipids, β-Gal on glycoproteins and glycolipids, α-1,2/3/6 Man on N-glycans, α-Fuc on N- or O-linked glycans.

Computer-Aided Methods for Analyzing Residues that could Affect Carbohydrate-Processing Enzyme-Glycan Complex Stability

Once a carbohydrate-processing enzyme(s) for conversion into a lectenz has been selected, its sequence can be analyzed for amino acid residues that, when mutated, could affect the affinity or stability or specificity of an enzyme-glycan complex.

In some embodiments, the sequence analysis can be performed by computational mutagenesis. In one embodiment, key protein residues affecting protein stability and/or ligand affinity can be identified using computational saturation mutagenesis experiments. The computational saturation mutagenesis is conducted at the amino acid sequence level and involves the replacement of one amino acid side chain by another, followed by computational analysis of the effect of the replacement on the affinity or stability or specificity of the interaction between the substrate and the enzyme. In other embodiments, targeted or random computational mutagenesis can be performed. In other embodiments the contribution made to the stability or the specificity of the enzyme-glycan complex by each amino acid in the enzyme can be computed directly for the glycan-enzyme complex.

In certain embodiments, “hotspots” key protein residues that affect protein stability or ligand affinity or ligand specificity are identified using computational alanine scanning mutagenesis (ASM). ASM can be performed by sequential replacement of individual residues by alanine. ASM can identify residues involved in protein function, stability and shape. Each alanine substitution examines the contribution of an individual amino acid to the functionality of the protein. A general overview of this technique is provided in Kollman, P. A., et al., Calculating Structures and Free Energies of Complex Molecules: Combining Molecular Mechanics and Continuum Models. Acc. Chem. Res., 2000. 33(12): p. 889-97 and Arakat, N., et al., Exploiting Elements of Transcriptional Machinery to Enhance Protein Stability JMB, 2007. 366(103-116). In a typical example, an MD simulation of the WT enzyme or enzyme-substrate complex is performed and the data collected. Subsequently, the side chains of all (or only selected) residues are truncated to the Cβ position, resulting in their conversion thereby to alanine. Any missing hydrogen atoms are added to form an intact alanine. This procedure is repeated for all of the structures collected in the MD simulation. Once converted to alanine, the effect of the mutation on the stability of the enzyme or the complex can be computed from the MD data. These effects are determined, by monitoring changes in the structure of the complex or protein, or by monitoring changes in the energies associated with the protein or complex. Separate MD simulations can also be performed after the alanine mutation has been introduced. Alanine scanning mutagenesis is not the only scanning mutagenesis method known in the art. Therefore, the discussion of this technique is exemplary only and not limiting of the present invention.

In yet other embodiments, key residues can be identified by the magnitude of their energetic contributions in the wild type complex, or by their proximity to the bound ligand. In one such embodiment, a subset of amino acid residues can be created that comprises identified “hotspot” key residues that directly interact with the substrate through hydrogen-bonds, van der Waals contacts, and/or through water mediated contacts. Any residue that is located no more than about 3.2 Å between non-hydrogen (or heavy) atoms can be considered to directly interact with the substrate through van der walls contacts or through hydrogen-bonds. Any residue that is located no more than about 4.5 Å between non-hydrogen atoms may be considered to interact with the substrate through water mediated contacts or non-specific electrostatic interactions. In other embodiments, the subset will comprise any residue that is identified as contributing less than at least about −0.5 kcal/mol to either the gas-phase (ΔE_{MM}) or total (ΔG) interaction energies.

Alternatively, the hotspots can be subjected to further theoretical analysis to predict either specific favorable mutations or identify classes (neutral, charged, hydrophobic, etc.) of potentially favorable mutations (class-focusing). Hayes, R. J., et al., Combining computational and experimental screening for rapid optimization of protein properties. Proc Natl Acad Sci USA, 2002. 99(25): p. 15926-31, describes the class-focusing technique.

In some embodiments, it might be preferred to avoid undue chance of degrading lectenz specificity. In these embodiments, residues that are directly involved in interactions with the substrate will not be initially selected for mutagenesis. In other embodiments, residues that interact non-specifically, but contribute significantly favorably to ligand binding (as identified by interaction energy calculations) will also be excluded from initial mutagenesis studies. All remaining residues, and particularly any that contribute unfavorably to binding, can then be considered for a first round of mutagenesis. In some embodiments, the previously excluded subset members can be subjected to mutagenesis after the initial round experimental and theoretical mutagenesis is complete.

### Computer-Aided Computational Simulation Methods for Predicting Carbohydrate-Processing Enzyme-Glycan Complex Binding Energies

In some embodiments, it will be important to predict the effect of the theoretical mutations performed by any of the computational mutagenesis methods described above on the binding energies of the carbohydrate-processing enzyme-glycan complex. In certain embodiments, the binding energies can be calculated using a free energy perturbation method, also known as thermodynamic integration (TI). TI can be used to quantify the energetic contributions to binding of key structural moieties. Straatsma, Holonomic Constraint Contributions to Free Energy Differences from Thermodynamic Integration Molecular Dynamics Simulations. Chem. Phys. Lett., 1992. 196: p. 297-302, Zacharias et al., Inversion of Receptor Binding Preferences by Mutagenesis: Free Energy Thermodynamic Integration Studies of Sugar Binding to L-Arabinose Binding Proteins. Biochemistry, 1993. 32: p. 7428-7434, and Chipot and Kollman, Alternative Approaches to Potential of Mean Force Calculations: Free Energy Perturbation versus Thermodynamic Integration Case Study of Some Representative Nonpolar Interactions. J Comput Chem, 1996. 17(9): p. 1112-1131, describe the TI methodology. Although TI is generally limited to examining relative binding energies for very similar ligands, it is capable of quantifying the energetic contributions to binding of key structural moieties. In a typical example of a TI simulation, the simulation is performed under modified MD conditions, in which the free energy is computed for the theoretical process of converting the initial residue into the final one through a series of incremental steps, during which the percentage contribution from each state is varied. This non-physical process is performed by mathematically mixing the energy functions for each state and is sometimes referred to as computational alchemy.

In yet other embodiments, the total free energy of binding (ΔG) can be calculated by direct decomposition of the interaction energies between the substrate and the protein (the reactants). Direct ΔG calculations combine molecular mechanics (MM) energy estimates with continuum solvent models, such as Poisson Boltzmann (PB) or generalized Born (GB) that attempt to capture the desolvation free energy. These calculations generally require the additional contributions from conformational entropy to be separately computed.

By way of example, and not by way of limitation, in a typical MM-GB/PB calculation the free energy is computed for the protein (ΔGₚᵣₒₜₑᵢₙ), ligand (ΔG_{ligand}), and complex (ΔG_{complex}) for each structural “snapshot” extracted from the MD trajectories. Depending on the enzyme of interest, the initial portion of the data is discarded to allow the system to equilibrate. For example, in a 5 ns trajectory, the first 1 ns can be discarded. In these models, snapshots of data can be collected at set intervals. By way of illustration only, 2,000 snapshots can selected (at 2 ps intervals) from the remaining 4 ns for molecular mechanical (MM) binding energy analysis. The binding free energy (ΔG) can then be computed by subtraction (see Equation 6). Averaging over the entire trajectory results in the final average interaction energies (<ΔG_{bind}>):

<ΔG>=<ΔG_{complex}>−<ΔGₚᵣₒₜₑᵢₙ>−<ΔG_{ligand}>, [6]

where the averaging is over the MD snapshots.

The free energies of the components can be computed by separating the energies into three categories, namely molecular mechanical (electrostatic and van der Waals), solvation, and entropic (see Equation 7):

<ΔG>=<ΔE_{MM}>−T<ΔS_{MM}>+<ΔG_{Solvation}> [7]

Prior to the analyses, the water molecules can be removed from the solvated trajectories. The energy contribution from solvation can then be obtained through application of the generalized Born (GB) implicit solvation model, which due to its relative speed, is well suited for application to large protein-carbohydrate complexes. The MM-GBSA results compare well with those from the more rigorous MM-PBSA analysis (based on the Poisson-Boltzman implicit solvent approximation). The GB approximation has also been shown recently to work well in computational alanine scanning. In at least one embodiment, the GB method for computing carbohydrate-protein interaction energies employs the GB parameterization of Tsui and Case, Theory and Applications of the Generalized Born Solvation Model in Macromolecular Simulations. Biopolymers, 2001. 56: p. 275-291.

In certain embodiments, vibrational, translational, and rotational contributions to the entropy can be derived from a normal mode analysis of the energy-minimized coordinates, while the conformational entropy is estimated from an analysis of the covariance matrix of the relevant internal coordinates. See Karplus and Kushick, Method for Estimating the Configurational Entropy of Macromolecules. Macromol., 1981. 14: p. 325-332. In the case of carbohydrates, it is particularly convenient and appropriate to focus on the conformational entropy associated with the inter-glycosidic torsion angles. Changes in conformational entropy, arising primarily from hindered rotations, can be estimated from the motions of the backbone torsion angles in the free and bound forms of each oligosaccharide. From the determinants of the covariance matrices for the torsion angles in the bound and free states the relative conformational entropies can be derived.

In some embodiments, the binding energies are calculated using a classical mechanical force field. Generally, the inter-atomic properties pertinent to the molecules involved are parameterized into the force field. To use the AMBER force field, the values for the parameters of the force field (e.g. force constants, equilibrium bond lengths and angles, charges are inputted). A fairly large number of these parameter sets exist, and are described in detail in the AMBER software user manual. Each parameter set has a name, and provides parameters for certain types of molecules.

In one embodiment, the binding analysis is conducted using GLYCAM/AMBER carbohydrate force field. The GLYCAM06 parameters can be used with a number of biomolecular force fields. Examples of force fields compatible with GLYCAM06 include, but are not limited to, AMBER, CHARMM, NWCHEM, etc. In certain embodiments, the GLYCAM parameters can be augmented by the AMBER parameters for proteins. GLYCAM06 does not employ any default or generic parameters and is no longer limited to any particular class of biomolecules, but is fully extendible in the spirit of a small-molecule force field. GLYCAM06 parameters are described, for example, in Kirschner et al., GLYCAM06: A Generalizable Biomolecular Force Field. Carbohydrates. J. Comput. Chem., 2007. Early View (DOI 10.1002/jcc.20820).

In certain additional embodiments, the computational simulation is performed to achieve conformational sampling. Such techniques include molecular dynamics simulation, Monte carlo simulation, or side-chain rotamer searching.

### Expression and Testing of Carbohydrate-Processing Enzyme Mutants

Upon identification of carbohydrate-processing enzyme mutants with predicted desirable ligand binding characteristics using computational mutagenesis and molecular simulations methods described herein, the affinity and complex stability predictions can be confirmed using experimental mutagenesis. In some embodiments, the coding sequence of a carbohydrate-processing enzyme of interest is amplified from genomic DNA isolated from a suitable species and subcloned into a suitable vector. Routine methods of gene cloning and protein overexpression have been described. The coding sequence from genomic DNA for a carbohydrate-processing enzyme of interest can be isolated from the chosen species and subcloned into any suitable vector. In some embodiments, the vector can be engineered to express a carbohydrate-processing enzyme of interest together with a suitable affinity tag. Tagging of the protein will facilitate its purification using affinity chromatography techniques. In one embodiment, a carbohydrate-processing enzyme can be tagged with a hexahistidine tag. In another embodiment, the carbohydrate-processing enzyme can be engineered to contain an antigen peptide tag. Examples of suitable vectors include, but are not limited to, pOPH6, pET, and pBAD. The pOPH6 can be transformed into the chosen E. coli strain for expression. The present invention is not limited to a particular strain of E. coli for overexpression of a protein.

Examples of suitable strain include DH5α.

Overnight cell culture (5-10 ml) can be inoculated into a suitable amount of nutrient broth (e.g., Luria-Bertani broth) containing adequate amounts of carbon source, minerals, ions, antibiotics, and other reagents. Generally, these batch productions are small scale, i.e., 100-200 ml, but larger volume batches can be prepared. Selection of antibiotics will depend on the engineered resistance of the E. coli strain and cloned vector. For example, for a pOPH6 vector cloned into DH5a one might use a Luria-Bertani broth containing 80-120 μg/ml ampicillin, 0.8-1.5% v/v glycerol, 80-150 mM potassium phosphate (pH 7.0), and 0.2-1.5 mM isopropyl β-D-1-thiogalactopyranoside (IPTG). The culture media can be harvested after 4-25 hrs of induction.

In some embodiments, overexpressed enzyme can be isolated. A variety of methods are available for protein purification. In one embodiment, clarified media containing over expressed glycosidase can be passed through an appropriate affinity column. A hexahistidine affinity tagged protein can be purified using a resin immobilized with nickel or cobalt. For example, clarified media can be diluted with cold loading buffer containing an appropriate amount of salt and imidazole, and passed through a Hi-Trap IMAC column (Amersham Pharmacia Biotech) at 2 ml/min. An antigen-tagged protein can be purified by passing it through a resin immobilized with an antigen-specific antibody. The recombinant enzyme can be desalted using an appropriate gel filtration column.

In certain embodiments, experimental mutagenesis is conducted using site-directed mutagenesis according to established protocols. By a way of example, site-directed mutagenesis is performed on the vector comprising the gene for an appropriate glycosidase (e.g., pOPH6) using the QuikChange™ mutagenesis kit from Stratagene (La Jolla, Calif.). The sense and antisense primers for each mutant can be designed based on the sequence of the gene and can be synthesized by an automated DNA synthesizer (Integrated DNA Technologies, Inc.). About 10 to about 20 ng of the plasmid and about 5 to about 20 pmole of sense and antisense primers can be added to the polymerase chain reaction (PCR) mixture, as per manufacturer protocol. PCR can be performed on a thermocycle control unit (MJ Research, Cambridge, Mass.). The DNA template can be digested by the addition of an appropriate endonuclease, as per manufacturer protocol. In some embodiments, the full coding region of each mutant will be fully sequenced to confirm that only the desired mutation is generated. In some embodiments, confirmed mutant DNAs can be used as a template to create the multiple mutations by the same procedure as used in the single amino acid mutation.

Once amino acids are mutants identified via computational methods for saturation mutagenesis, a library of mutant proteins can be screened for mutants for desirable binding characteristics. A number of technologies used for high throughput screening of protein-ligand interactions are available in the art. Examples of such technologies suitable for the present invention include, but are not limited to, two-hybrid system, mRNA display, phage display, yeast display, ribosome display, and bacterial display. The approach of the present invention provides an additional subjective way to identify sites in the protein that should be randomized in the library. Thus, by combining the computational analysis of the present invention, one is able to design and then construct a focused biocombinatorial library. Such libraries by their design and construction provide a far more efficient approach for library screening.

In some embodiments, high throughput screening of protein-ligand interactions can be performed by creating a mutagenic display library. One such library system can be synthesized by GENEART. In one embodiment, the library can be displayed on a phage. The phage display library can be constructed using protocols well-established in the art. By way of example, the DNA library encoding the protein or peptide of interest is ligated into the pIII or pVIII gene of M13 filamentous phage. The phage gene and insert DNA hybrid is then transformed into E. coli bacterial cells such as TG1 or XL1-Blue E. coli. If a “phagemid” vector is used (a simplified display construct vector) phage particles will not be released from the E. coli cells until they are infected with helper phage, which enables packaging of the phage DNA and assembly of the mature virions with the relevant protein fragment as part of their outer coat on either the minor (pIII) or major (pVIII) coat protein. The incorporation of many different DNA fragments into the pIII or pVIII genes generates a library from which members of interest can be isolated. By immobilizing a relevant DNA or protein target(s) to the surface of a well, a phage that displays a protein that binds to one of those targets on its surface will remain while others are removed by washing. Those that remain can be eluted, used to produce more phage (by bacterial infection with helper phage) and so produce a phage mixture that is enriched with relevant (i.e. binding) phage. The repeated cycling of these steps is referred to as ‘panning’, in reference to the enrichment of a sample of gold by removing undesirable materials.

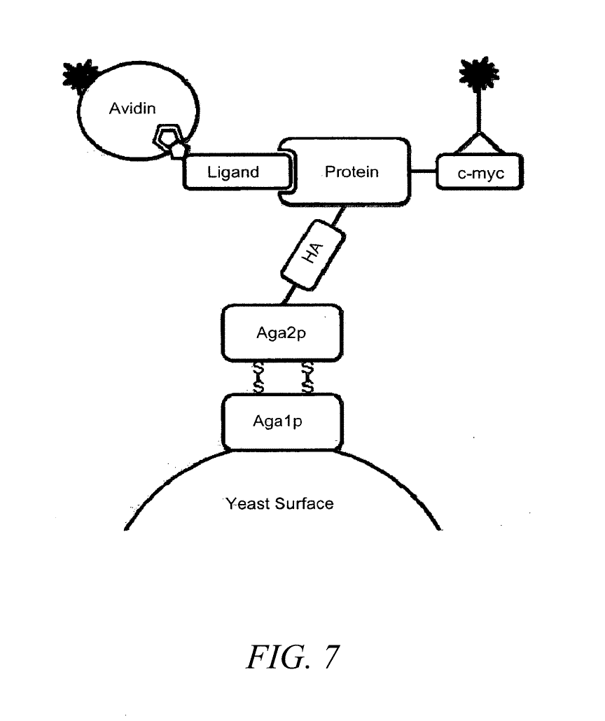

In other embodiments, the mutagenic display library can be displayed on yeast. In yeast display, a protein of interest can be displayed as a fusion to the Aga2p protein on the surface of yeast. The Aga2p protein is naturally used by yeast to mediate cell-cell contacts during yeast cell mating. As such, display of a protein via Aga2p projects the protein away from the cell surface, minimizing potential interactions with other molecules on the yeast cell wall. See FIG. 7, which illustrates the expression vector as a fusion of the Aga2 gene, a HA (hemagglutinin) tag, the displayed protein, and the c-myc tag. A yeast display library can be constructed using protocols well-established in the art. In some embodiments, the gene of interest can be cloned into a vector of choice in frame with the AGA2 gene. Examples of suitable vectors include, but are not limited to, pYD1 vector (Invitrogen) and pPNL6 (Pacific Northwest National Laboratory). The resulting construct is then transformed into suitable yeast strain (e.g., EBY100 S. cerevisiae) containing a chromosomal integrant of the AGA1 gene. Expression of both the Aga2 fusion protein from the vector and the Aga1 protein in the EBY100 host strain is regulated by the GAL1 promoter, a tightly regulated promoter that does not allow any detectable cloned protein expression in absence of galactose. Upon induction with galactose, the Aga1 protein and the Aga2 fusion protein associate within the secretory pathway, and the cloned mutant is displayed on the cell surface.

Once a mutagenic library displaying mutated carbohydrate-processing enzymes on cell surfaces is constructed, it can be screened to identify mutants that have desirable binding and complex-formation properties. The basic principle of the assay system used to identify mutants that are capable of high-affinity complex formation with a ligand of choice involves preparing a reaction mixture containing the display library and the ligand under conditions and for a time sufficient to allow the two reagents to interact and bind, thus forming a complex. The formation of any complexes between the binding partners is then captured. After the reaction is complete, unreacted components are removed (e.g., by washing) and any complexes formed will remain immobilized on the cell surfaces. The detection of complexes anchored on the cell surface can be accomplished in a number of ways. In some embodiment, the ligand can be pre-labeled, either directly or indirectly. Where the ligand is labeled, the detection of label immobilized on the cell surface indicates that complexes were formed. Where the binding partner is not pre-labeled, an indirect label can be used to detect complexes anchored on the surface. Labeling of molecules is well known, for example, a large number of biotinylation agents are known, including amine-reactive and thiol-reactive agents, for the biotinylation of proteins, nucleic acids, carbohydrates, carboxylic acids. A biotinylated substrate can be attached to a biotinylated component via avidin or streptavidin.

In some embodiments, the size of the display library can be enriched to comprise yeast that bind biotinylated N-linked glycopeptides with low to high affinity. Low affinity interactions are difficult to measure directly. By immobilizing biotinylated N-linked glycopeptides to the surface of streptavidin coated paramagnetic beads (e.g., Invitrogen), the library can be enriched for yeast displaying proteins that binds to the target on the bead surface. The yeast captured by the N-linked glycopeptide coated paramagnetic beads are isolated with a magnet, nonbinding yeast washed away, and the panning process repeated. In some embodiments, the library can be reduced to 10⁶-10⁸ cells depending on the initial size of the library and number of rounds of panning. In at least one embodiment, the library is reduced to about 1×10⁷.

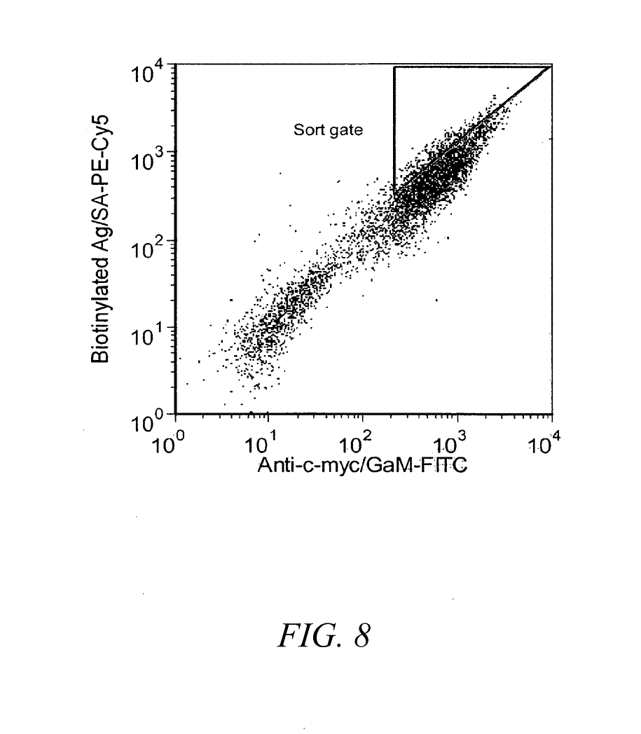

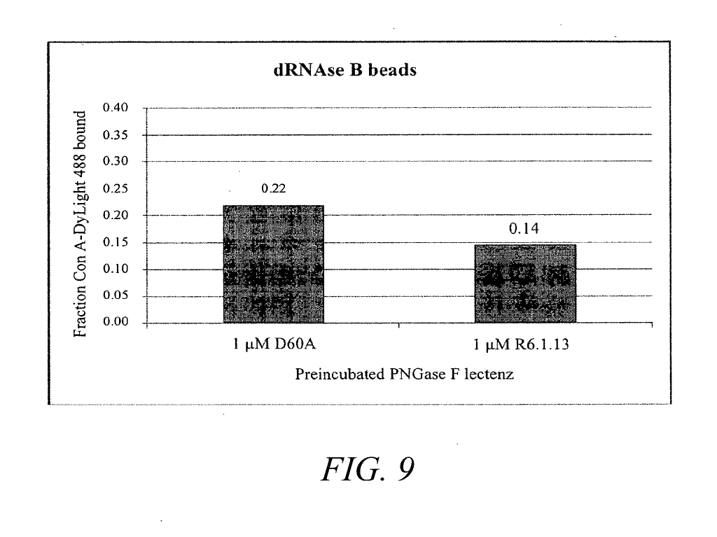

In certain embodiment, an initial screening of high-affinity mutants can be conducted. In these embodiments, the screening can be done by flow cytometry. The screening can be done by selecting only the mutants that bind to specific biotinylated glycopeptides. By a way of example, and as detailed in FIG. 9, the yeast expressing the Aga2p fusion protein with a C-terminal c-myc tag can be incubated with anti-c-myc mAb, followed by an addition of a fluorescent secondary mAb to detect the yeast that have expressed full-length glycosidase clones. In certain embodiments, detection of mutants that bind to the biotinylated glycopeptides can be accomplished by addition of streptavidin. In one embodiment, streptavidin can be labeled with a suitable fluorescent label (e.g., PE-Cy5). Streptavidin-PE-Cy5 can be used to fluorescently label the enzyme clones that bind the glycopeptides. Only the cells expressing c-myc that have a high affinity for the glycopeptides will be sorted (FIG. 8). In some embodiment, the dissociation constants of selected glycosidase mutants displayed on individual yeast clones can be determined. In at least one embodiment, the dissociation constant can be determined by flow cytometry.

In certain embodiments, high affinity binders identified in the preliminary screening assay can be expressed and purified for further binding studies. Binding affinities of generated lectenz can be analyzed by a variety of techniques known in the art (e.g., filter binding assay, electrophoretic mobility shift assay (EMSA), surface plasmon resonance (SPR), etc.). In one embodiment, binding constants are measured by SPR using a Biacore apparatus. A typical assay used to evaluate binding constants of a complex using SPR includes immobilization of a lectenz of interest on an SPR chip surfaces at 20-30° C. by a suitable coupling method (e.g., amine-coupling method), with mock-derivatized flow cells serving as reference surfaces. The binding analyses can be performed at various temperatures with continuous flow (10-50 μl/min) of running buffer. The running buffer can be 15-25 mM Tris-HCl (pH 7.5), 100-200 mM NaCl, 0.5-1.5 mM EDTA, 0.0025-0.0075% P20 detergent. Ligands can be prepared by serial dilution in the respective running buffers in to obtain an appropriate concentration range. The binding of ligand can be analyzed in a concentration series (0.625-10 μM) over a low-density immobilization surface of lectenz. The maximal equilibrium sensogram values can be used to plot a saturation binding curve and calculate values for the equilibrium dissociation constant (K_{d}) directly.

In one aspect of the present invention, the method for generating lectenz further comprises using crystallographic data for enzyme-substrate or enzyme inhibitor complexes as the basis for computational mutagenesis. In general, crystallization and crystal growth parameter optimization of the lectenz with substrates can be performed by the methods and procedures described in the art. Conditions suitable for crystallization will be determined on a case-by-case basis. Crystals can be tested for diffraction and the crystals which diffract to the highest resolution can be used for data collection. In certain embodiments, Molecular Replacement will be used to solve the structure of the complexes. In these embodiments, X-ray data of generated lectenz-glycan complexes can be used to validate the MD simulations and/or to initiate new simulations.

Although exemplified throughout the present invention in terms of a lectenz derived from a glycan processing enzyme, it is understood that the present invention is broadly applicable to any enzyme-substrate complex. Thus, without being limited to the following examples, and simply to further exemplify the scope of the present invention, the lectenz approach can be used to convert enzymes such as proteases, lipases, kineases, phosphatases, hydrolases, isomerases, and others, to receptor proteins maintaining specificity for the enzyme substrate.

Moreover, the present invention is not limited to carbohydrate processing enzymes. It is further applicable to carbohydrate binding proteins. Indeed, it is applicable to protein-ligand interactions in general.

### Methods of Use

Another aspect of the present invention provides methods of using lectenz described herein. The vast number of potential applications of lectenz described herein will be immediately apparent to persons skilled in the art. Below are but a few embodiments describing potential utilities of such reagents.

In certain embodiments, lectenz of the present invention can be used for application in glycan-specific analytical tools. Lectenz-based glycan-specific analytical tools of the present invention have potential use as a method of detection in many areas, including environmental, fermentation, food and medical areas and could be used for in vivo or in vitro sensing in humans or animals.

In some embodiments, lectenz with defined carbohydrate specificity described herein can be used to interrogate biological samples in the search for abnormal glycosylation. Examples of biological samples include, but are not limited to, any biological fluid, tissue, or organ. Examples of the biological fluids include, but are not limited to blood, urine, serum, saliva, cerebra-spinal fluid, and semen. In other embodiments, lectenz of the present invention can be used for a detection of a target carbohydrate-based analyte level in biological fluids. Examples of the target analytes include, but are not limited to, endogenously found molecules, such as N- or O-linked glycans, glycosaminoglycans (including heparin), exogenously consumed species, such as plant polysaccharides, carbohydrate-based drugs, and pathogens, whose surfaces are often coated in complex distinct glycans. In other embodiments, the lectenz described herein find their application in drug discovery and evaluation of biological activity of new glycan-based compounds.

In some specific embodiments, lectenz described herein can be used for diagnosing, and/or treating diseases manifested by abnormal glycosylation. In one embodiment, lectenz of the present invention can be used to detect certain tumor antigens comprising glycoproteins, glycolipids, and/or a variety of carbohydrate epitopes. A number of these tumor antigens have been found to be up-regulated in the neoplastic disease state. Examples of tumor antigens that can signal a development and progression of a neoplastic disorder, and that can be detected by lectenz of the present invention, include, but are not limited to, carcinoembryonic antigen (CEA), which is a glycoprotein associated with colorectal, gastric, pancreatic, lung, and breast carcinomas, and the developing fetus; carbohydrate antigen 19-9 (CA 19-9), or sialylated Lewis A antigen, which is present in a glycolipid found in patients with pancreatic cancer; and carbohydrate antigen 15-3 (CA15-3), associated with breast cancer.

The presence of the antigen does not necessarily indicate transformation to a cancerous cell, however, its localization in the cell is indicative, as in the case of CEA. For this reason, there is a need for highly selective and high affinity analytical tools. The diagnostic tests currently rely on antibodies that were often generated against the peptide portions of the glycoprotein or sugar portions of glycolipid, however, the exact epitopes are only now being defined. In the examples in which the glycans have been characterized, multiple glycoforms are often present (CEA, for example). Lacking reagents that are able to discriminate between glycoforms, it is currently impossible to determine the extent to which subtle variations in glycosylation correlate with disease state, cancer type, or tissue localization. At present, these questions can be addressed primarily by MS analyses of isolated glycoproteins, which are examined as mixtures of glycoforms. Typically, the only level of glycoform-focusing that is performed is the enrichment in high-mannose containing glycans using lectin (concanavalin A, (Con A)) affinity chromatography. More efficient laboratory analyses and routine clinical diagnostic techniques remain severely limited by the lack of glycoform-specific reagents.

Lectenz of the present invention are particularly useful for quantifying the relative abundances of each glycoform present in any given glycoprotein in a biological sample. As used herein, the term “glycoform” refers to type of protein with a specific type of glycoprotein attached. Two proteins would be of the same glycoform if they carried the same glycoprotein. In some embodiments, lectenz of the present invention can be used to enrich the biological sample with a particular glycoform. In other embodiments, lectenz generated by the methods described herein can be used to identify specific glycosylation sites on the protein surface to which the glycans are attached. In these embodiments, lectenz specific for particular oligosaccharides will be used to separate intact glycopeptides from a proteolytic digest of any glycoprotein. For example, a PNGase-F derived lectenz can be used to separate N-linked glycopeptides from other glycopeptides or peptides, as might arise from a typical protease digestion of a glycoprotein. Enriching the sample in the analyte of interest is of great assistance in the further characterization of the glycopeptides fractions. In particular, enrichment facilitates the identification of the peptide sequence and the glycan structure, which can enable the identification within the intact protein of the glycosylation sites and the characterization of the particular glycans present at each glycosylation site.

In other embodiments, lectenz of the present invention will find their use in monitoring specific glycan modifications of proteins in biological fluids, tissues, organs, or living cells. Lectenz engineered by the method of the present invention will not depend on the identity of the protein, that is they will be context independent, and will be able to recognize any protein that comprises a given glycan, and therefore will be very useful for detection of given glycan modifications.

In yet other embodiments, lectenz of the present invention can be used for in vitro or in vivo staining cells or tissues.

In other embodiments, the lectenz can be developed so as to be specific for a particular glycoprotein or glycosylation site in a glycoprotein. Such a lectenz could be employed to monitor a particular glycoprotein in a mixture, as might arise during the production of recombinant glycoproteins for use in the pharmaceutical or research industries.

In the foregoing embodiments, the lectenz can be tagged with a stain or a dye and applied to a biological sample comprising cells or tissues or glycoproteins or glycopeptides or oligosaccharides or polysaccharides of interest.

In certain embodiments, lectenz of the present invention can be used as therapeutic agents. In these embodiments, design of a particular lectenz can based on glycosidases for which human homologues exist. This will ensure that such lectenz lack immune reactivity. In certain embodiments, lectenz of the present invention can be modified for delivery of an active therapeutic agent. Since lectenz of the present invention have a defined glycan specificity, a delivery of the therapeutic agents can be targeted only to those cells, tissues, or organs that display a particular glycan. Examples of therapeutic agent that can be used for site-specific delivery include, but are not limited to, various chemotherapeutic, antibiotic, and antiviral agents, toxins, radioisotopes, cytokines, etc.

In certain embodiments, lectenz of the present invention can be used as reagents for affinity separation, including, for example, affinity chromatography. Affinity chromatography is a method of separating biochemical mixtures, based on a highly specific biological interaction such as that between lectenz and glycan. The present invention is not limited to any specific design or chromatographic system. In general, lectenz will be either covalently attached or otherwise immobilized to the solid support, and will constitute a stationary phase. In certain embodiments, the lectenz-derivativized stationary phase can be used in column chromatography. In these embodiments, the particles of the solid stationary phase will be used to fill the whole inside volume of the tube (packed column). Alternatively, the solid phase particles will be concentrated on or along the inside tube wall leaving an open, unrestricted path for a biological sample (i.e., the mobile phase) in the middle part of the tube (open tubular column). In other embodiments, the lectenz-derivativized stationary phase can be used for batch chromatography. In these embodiments, the stationary phase can be added to a vessel and mixed with the biological sample.

Glycoprofiling with Multiplexed Suspension Arrays

The present invention includes compositions and methods directed to the multiplexed analysis of carbohydrates and carbohydrate containing compounds. As used herein, the phrase “multiplex,” or grammatical equivalents, refers to the simultaneous detection of multiple analytes in a single assay. Multiplexed analysis is the ability to perform multiple discrete assays in a single tube with the same sample at the same time. In some embodiments of the multiplexed assays described herein, two, three, four, five, six, seven, eight, nine, ten, eleven, twelve, thirteen, fourteen, fifteen, sixteen, seventeen, eighteen, nineteen, twenty, or more analytes may be measured. In some embodiments, at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, or at least twenty analytes may be measured. In some embodiments, more than two, more than three, more than four, more than five, more that six, more than seven, more than eight, more than nine, more than ten, more than eleven, more than twelve, more than thirteen, more than fourteen, more than fifteen, more than sixteen, more than seventeen, more than eighteen, more than nineteen, or more than twenty analytes may be measured. In some embodiments, about ten, about twenty, about thirty, about forty, about fifty, about sixty, about seventy, about eighty, about ninety, about one hundred, or more analytes may be measured. In some embodiments, at least about ten, at least about twenty, at least about thirty, at least about forty, at least about fifty, at least about sixty, at least about seventy, at least about eighty, at least about ninety, or at least about one hundred analytes may be measured. In some embodiments, more than about ten, more than about twenty, more than about thirty, more than about forty, more than about fifty, more than about sixty, more than about seventy, more than about eighty, more than about ninety, or more than about one hundred analytes may be measured. In some embodiments, hundreds, or thousands of analytes may be measured.

Unlike protein sequences, which are encoded by the organism's genetic material, the subsequent attachment of complex carbohydrates (glycans) in eukaryotes is controlled by enzymes that either trim or extend the glycan core. A single protein frequently exhibits multiple versions of the glycan, depending on the age or location of the protein. Variations in the glycosylation pattern (glycoprofile) can also result from a range of diseases that introduce mutations into gene sequences, or that alter regulatory control pathways. Aberrant protein glycosylation is therefore a hallmark of several disease states, including diabetes (Coppo and Amore, 2004, Kidney International; 65(5):1544-1547), IgA nephropathy (Amore and Coppo, 2000, Nephron; 86(3):255-259), and various cancers (Krengel et al., 2004, J Blot Chem; 279(7):5597-5603). Because of their exposure on cell surfaces, the glycan chains frequently also serve as receptors for viral and bacterial pathogens (Lim et al., 2008 J. Proteome Res. 7(3):1251-63). The ability to characterize glycoprofiles is therefore relevant to disease marker discovery, the development of therapeutics, the study of infectious diseases, and glycobiology research in general.

The compositions and methods described herein utilize suspension array technology (SAT). With suspension array technology, an assay is carried out with the array elements suspended in a liquid or gel phase. The multiplex suspension assays described herein utilize an array of different carbohydrate binding molecules, each carbohydrate binding molecules with a known carbohydrate binding specificity, to obtain a glycoprofile of the carbohydrate structure(s) in a sample. As used herein, the term “carbohydrate,” also referred to herein as “glycan,” is meant to refer to an organic compound of a general formula Cₘ(H₂O)ₙ. Such multiplexed suspension arrays (MSA) will provide for the rapid, robust, and cost-effective characterization of glycosylation patterns. Such multiplexed suspension arrays for the characterization of glycosylation patterns are also referred to herein as “Glycoprofiling Multiplexed Suspension Arrays,” “glycoprofiling multiplexed suspension arrays,” “glycoprofiling multiplexed suspension arrays (MSA),” “glycoprofiling MSA,” “multiplexed suspension arrays glycoprofiling,” “multiplexed suspension arrays (MSA) glycoprofiling,” “MSA Glycoprofiling,” or “GlycoProf MSA™.”

Each carbohydrate binding molecule of a given specificity is linked to the external surface of a population of individually addressable particles. Preferably individually addressable microspheres such as beads or nanoparticles are employed. Normally the surface of each bead is functionalized with a single type of carbohydrate binding molecule, although in some embodiments a bead can be functionalized with two or more types of carbohydrate binding molecules. The array elements in suspension array technology are modular and suspended in a liquid or gel; typically the array elements take the form of individual particles.

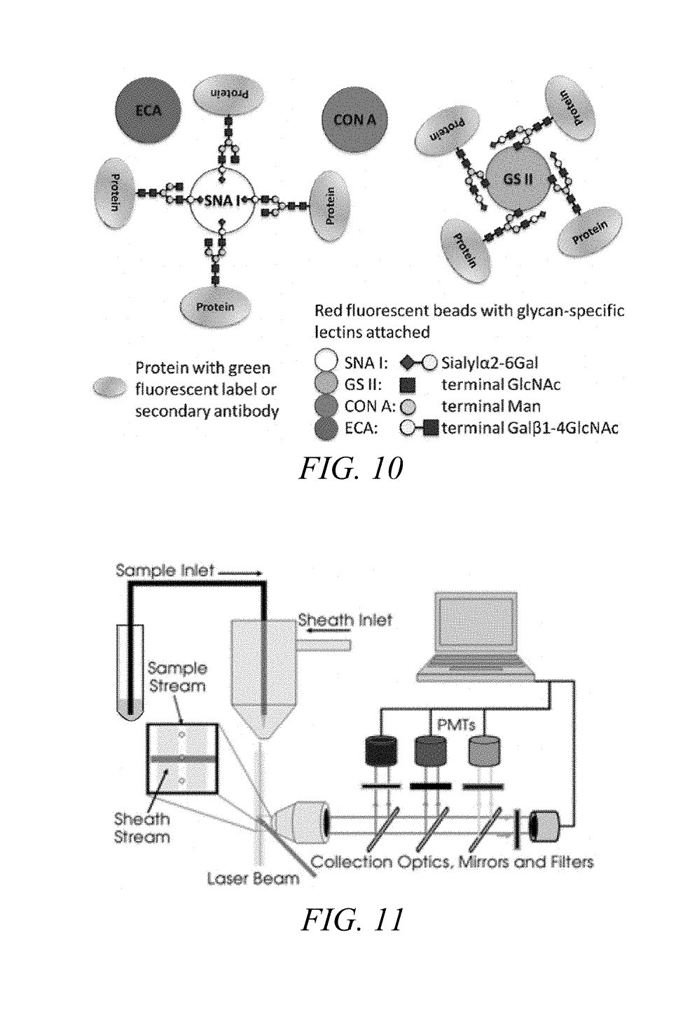

By judicious choice of carbohydrate-specific reagents, the glycoprofiling multiplexed suspension arrays described herein provide a simple but robust technology, able to resolve such differences as the termination state of glycan sequences. FIG. 10 shows a schematic representation of the multiplexed interactions between multiple suspension array technology (SAT) reagents and a glycoprotein analyte. In the embodiment shown in FIG. 10, glycan specific lectins are conjugated to red fluorescent multiplex microspheres (beads), and then incubated with a green fluorescently labeled glycoprotein.

The MSA glycoprofiling approach described herein combines suspension array technologies (SAT) with established high-throughput detection. Any of a variety of detection methods and addressable particles may be used, such as, for example, any of those reviewed in more detail in Braekmans et al., 2002, Drug Discovery; 1:447-456; Wilson et al., 2006, Agnew Chen Int Ed; 45:6104-6117; and Birtwell and Morgan, 2009, Integr Biol; 1:345-362 (which are herein incorporated by reference in their entireties).

In some embodiments, binding detection in SAT methods employs target-specific receptors that are conjugated to the surface of microspheres (beads) with distinct optical properties, such as light scatter based, for example, on bead size or granularity, and/or fluorescence from an internal agent. A fluorescent agent includes, for example, a fluorescent dye, quantum dots, and surface-enhanced raman scattering (SERS).

Any of a variety of protein-attachment chemistries may be used for attachment to an addressable particle, ranging from, for example, physical adsorption or covalent coupling, to specific noncovalent attachment using affinity tags (poly-his, biotin, glutathione-S-transferase, etc.).

The binding of a carbohydrate, carbohydrate containing compound, or glycoprotein bound to each bead maybe determined with the use of a secondary binding agent or an affinity partner with a binding specificity for the analyte, carbohydrate, carbohydrate containing compound, or glycopeptide being assayed. Such a secondary binding agent or affinity partner may be detectably labeled, for example a labeled antibody. Such an antibody may be labeled with, for example, a fluorophore, biotin, or an enzyme. A biotin-streptavidin based detection scheme may be used. Flurophores include, for example, fluorescent dyes such as phycoerythrin (PE), one of the many ALEZA FLUORs, and reactive water soluble fluorescent dyes of the cyanine dye family, such as Cy2, Cy3, or Cy5. See, for example, “Antibody labeling from A to Z,” Invitrogen 2008 (available on the world wide web at invitrogen.com/etc/medialib/en/filelibrary/cell_tissue_analysis/pdfs.Par.60486.File.dat/B-075469-Zenon%20Brochure-flr.pdf). Alternatively, the carbohydrate or glycopeptide being assayed may be directly labelled with such a detectable label.

While a variety of detection methods may be employed, including, but not limited to flow cytometry, image based systems, and microscope based systems. In some embodiments, an image based system may be used. Examples include, but are not limited to, Luminex's MAGPIX (see luminexcorp.com/Products/Instruments/index.htm), Amnis's ImageStream (see amnis.com/documents/brochures/ImageStreamx_brochure.pdf) and spectral flow cytometer (see onlinelibrary.wiley.com/doi/10.1002/cyto.a.20706/full), and Nexcelom Biosciences' Cellometer.

In some embodiments, flow cytometry is a preferred detection method. Flow cytometry is a powerful platform for high-throughput and quantitative functional analysis of cells, and of purified proteins and other biomolecules using microspheres. Flow cytometry rapidly measures the fluorescence and other optical properties of individual particles. The basic principles of flow cytometry, as well as the numerous variations, have been well described (Shapiro H M. Practical Flow Cytometry. 4th. New York: Wiley-Liss; 2004). See also, Nolan and Sklar, 1998, Nat Biotechnol; 16: 633-638; Nolan et al., 2006, Curr Protoc Cytom; Chapter 13: Unit 13.8; Yang and Nolan, 2007, Cytometry A; 71(8):625-31; and Nolan and Yang, 2007, Brief Funct Genomic Proteomic; 6(2):81-90.

In a typical flow cytometer (FIG. 11), sample is carried in a sheath stream through a laser beam where fluorescent dyes are excited. The emitted fluorescence is collected, spectrally filtered and detected using photomultiplier tubes. Flow cytometry provides for high speed single particle analysis and selection. Samples are hydrodynamically focused to a very thin sample stream, typically on the order of 10 μm in diameter. This focused sample stream is passed through a focused laser beam on the order of 10 μm in height. The intersection of the sample stream and laser beam (FIG. 11, inset), often called the probe volume, has dimensions of ˜10 μm³, or about 1 pl. Under these conditions, in a typical mammalian cell (diameter ˜10 μm) suspension, cells will be lined up single file and will pass one at a time through the probe volume, where fluorescence and light scatter signals are collected. Typical transit times through the probe volume are 10 μs or less for many commercial flow cytometers, enabling sample analysis rates of thousands of cells or beads per second. High speed cell sorters are capable of analysing tens of thousands of cells or beads per second (Ibrahim and van den Engh, 2003, Curr Opin Biotechnol; 14:5-12), and sorting selected sub-sets of cells or beads into tubes or microwell plates. Because the measurement probe volume is small, background signal, which often limits sensitivity, is low, making flow cytometry an especially sensitive fluorescence detection platform. While custom instruments have reported single molecule sensitivity (Keller et al., 2002, Anal Chem; 74:316A-324A; and Habbersett and Jett, 2004, Cytometry A; 60:125-34.3), most commercial cytometers have detection limits of a few hundred molecules of a small organic fluorophore such as fluorescein. Intensity standards and calibration protocols have been developed that allow fluorescence measurements to be expressed in absolute units of molecules per cell (Habbersett and Jett, 2004, Cytometry A; 60:125-34; Schwartz et al., 1996, Cytometry; 26:22-31; Schwartz et al., 1998, Cytometry; 33:106-14; Schwartz et al., 2004, Cytometry B Clin Cytom; 57:1-6; Wood and Hoffman, 1998, Cytometry; 33:256-9). These approaches consider instrument response, the properties of reagents used (the fluorophore to protein ratio of an antibody, for example), and spectral matching between calibrators and unknowns. Such absolute quantification facilitates assay development and mechanistic studies, and is critical for certain clinical applications.

Flow cytometry can make high speed, quantitative optical measurements of multiple fluorophores simultaneously. The simplest bench top instruments typically measure three or four colors of fluorescence excited by a single laser. Additional lasers and detectors enable the detection of additional fluorophores, and the past decade has seen a steady increase in the number of parameters measured (De Rosa et al., 2001, Nat Med; 7:245-8; Roederer et al., 1997, Cytometry; 29:328-39), such that three laser eight color experiments are not uncommon, and 19 parameter (fluorescence plus light scatter) measurements have been reported (Perfetto et al., 2004, Nat Rev Immunol; 4:648-55). The high information content provided by multiparameter measurements not only allows for more efficient analysis of samples, it is required to identify key sub-populations present in a complex mixture of cells. Because the probe volume in the flow cytometry measurement is small, signal from free fluorophore is often negligible, allowing samples to be measured without a wash step. In addition, homogeneous assays enable continuous kinetic resolution, allowing flow cytometry to be exploited for real-time mechanistic studies of biochemical processes. Such wash-less assays enable streamlined sample processing and are especially amenable to automated analysis.

Cytometric measurements (fluorescence channel) may be calibrated in terms of mean equivalent soluble fluorescein molecules (MESF) using calibrated FITC-labeled microspheres. Standard curves may be generated. Commercial software is available to for assist with data analysis. The prototypical multiplexed bead-based analysis is the antibody sandwich assay. Essentially, an ELISA performed on a microparticle instead of a microwell bottom, an immobilized antibody captures an analyte from a complex sample, and a labeled reporter antibody completes the sandwich allowing the analyte to be quantified via the fluorescence intensity of the microsphere. The principles and considerations for developing such multiplexed assays have been described in detail (Camilla et al., 2001, Clin Diagn Lab Immunol; 8:776-84; Carson and Vignali, 1999, J Immunol Methods; 227:41-52; Kellar et al., 2001, Cytometry; 45:27-36). In general, the bead-based assays offer sensitivity comparable to the standard colorimetric ELISA, with the advantages of smaller sample size, fewer processing steps, which combined with the efficiency of multiplexing constitute an extremely powerful approach to the detection of soluble proteins.

In terms of convenience, and cost, it is important to note that the most basic benchtop flow cytometers, with one or two lasers and four or five detectors, are capable of making sensitive (a few hundred to a few thousand molecules) and quantitative measurements of multiple different fluorescent probes simultaneously on individual particles. In the presently available systems, a 100-plex SAT assay can be performed approximately every 30 seconds. Current flat array technologies employ 11-45 target-specific receptors, which is within the current dynamic range of flow cytometry based SAT. Running continuously, such systems could process 288,000 assays per day, allowing the MSA glycoprofiling approach described herein to be used for real-time process monitoring, as well as for the analysis of large numbers of samples, as for example in a regulatory laboratory.

The MSA glycoprofiling approach described herein may make use of individually addressable particles. Such individually addressable particles include, for example, microspheres and nanoparticles. In preferred embodiments, individually addressable particles are optically encoded microspheres; microspheres with distinct optical properties, such as light scatter or fluorescence from an internal dye. Based on a dye color coded scheme, 100 or more distinct sets of optically encoded microspheres, also referred to as color coded beads, can be produced. Because of the dye ratio incorporated each bead, each unique bead population can be analyzed separately when lasers are used to excite the internal dyes that identify each microsphere particle. Each bead set will have a separate capture reagent, such as a separate carbohydrate binding molecule, attached to the surface, allowing for the capture and detection of specific analytes from a sample. Encoded microspheres and flow cytometry have been employed for a wide range of multiplexed molecular analysis, and detailed protocols for many of these have been developed. See, for example, Fulton et al., 1997, Clin Chem; 43:1749-56; Kettman et al, 1998, Cytometry; 33:234-43; and Oliver et al., 1998, Clin Chem; 44(9):2057-60. Encoded microspheres are commercially available from a number of sources, including, for example, Spherotech (Lake Forest, Ill.).

Each derivatized batch of microspheres (array element) may be prepared in bulk, and by virtue of the solution phase chemistry employed for conjugation, the receptors are dispersed evenly over the surface of the sphere. Because the target-receptors are conjugated to beads, the elements of the array may be combined and altered at will. Arrays with particular reagents may be created that target the interests of a particular research community, a particular pharmaceutical company, or a Federal regulatory body. In addition, SAT analyses may be performed on any flow cytometer (FIG. 11), without the need to dedicate it to SAT use. The use of flow cytometry has some very significant advantages in terms of statistical precision and reproducibility over flat array technologies.

With the MSA glycoprofiling approach described herein, each bead set will have a separate capture reagent, such as a separate carbohydrate binding molecule, attached to the surface, allowing for the capture and detection of specific analytes from a sample. Carbohydrate binding molecules include, but are not limited to, lectins, antibodies, LECTENZ molecules (carbohydrate processing enzymes that have been inactivated but still bind to carbohydrate(s) with high specificity), carbohydrate-binding proteins, carbohydrate binding domains of proteins, pathogen adhesion domains (such as cholera toxin B, other toxins, and hemagglutinin), aptamers including protein, RNA or other small molecule aptamers, and any other molecule that naturally binds or is engineered to bind a carbohydrate.

Lectins are widely used carbohydrate-binding molecules for glycoprofiling. Any of a variety of lectins (sugar-binding proteins), including, but not limited to, any of those described herein, may serve as a carbohydrate binding molecule. Lists of representative carbohydrate binding lectins are also included in the examples provided herewith. Lectins are not, however, ideal reagents. They are not generally high affinity, and some lectins display relatively broad specificity, or context dependency. As an illustration, the lectin MAL II which is known to prefer Sialylα2-3Gal linkages, displays strong context dependence; an examination of the CFG binding data indicates that MAL II will bind to the linear sequence Sialylα2-3Galβ1-4GlcNAcβ1-3Galβ1-4GlcNAcβ1-3Galβ1-4GlcNAcβ, but will not recognize the related branched sequence Sialylα2-3(Galb1-3GalNAcb1-4)Galb1-4Glcb. In direct contrast, the carbohydrate binding B domain from cholera toxin (CTB) binds the branched sequence, but not the linear. The CFG glycan array data provides an unrivalled source of experimental specificities from which to select reagents with well-defined specificities. Thus, to enhance the robustness of a glycoprofiling MSA methods, redundant MSA reagents may be employed, such as the lectins PSL and SNA I, both of which bind to Sialylα2-6Gal linkages.

In addition to lectins, other well-characterized carbohydrate-detection reagents, such as pathogen adhesion domains and antibodies, may serve as carbohydrate binding molecules. A carbohydrate binding molecule may be an antibody with a binding specificity for a carbohydrate determinant. Such antibodies, include, but are not limited to, any of those described herein. Lists of representative carbohydrate binding antibodies and lectins are also included in the examples provided herewith. Anti-carbohydrate antibodies provide an alternative to lectins, but they are also known to display cross-reactivities with dissimilar glycans. For these reasons, reagents with redundant binding properties will be employed for a robust glycoprofiling technology.

One or more of the antibodies or lectins employed as carbohydrate-specific receptors for glycoprofiling with microarrays may be used in the multiplex suspension array glycoprofiling approach of the present invention. See, for example, Chandrasekaran et al., 2002, Glycobiology; 12(3):153-162; Davidson et al., 2000, Hum Pathol; 31:1081-1087; and Prien et al., 2008, Glycobiology; 18(5):353-366.

Carbohydrate binding molecules used in the MSA glycoprofiling approach of the present invention include carbohydrate processing enzymes that have been inactivated but still bind to carbohydrate(s) with high specificity. Such molecules, also referred to herein as a “LECTENZ” molecule, a “Lectenz®” molecule, or a “lectenz,” include a catalytically inactive mutant of a carbohydrate-processing enzyme that has substantially the same specificity for a given glycan as the wild-type enzyme, and an increased affinity towards the glycan as compared to the WT enzyme. As used herein, the term “substantially the same” is meant to describe a specificity of the glycosidase mutant that is at least 60% of the wild-type enzyme. In some embodiments, the specificity of the mutant is at least 70% of the WT enzyme. In at least one embodiment, the mutated glycosidase is at least 85% as specific to its substrate as the wild-type enzyme to the same substrate. In other embodiments, the mutated glycosidase is at least 95% as specific to its substrate as the wild-type enzyme to the same substrate. LECTENZ molecules are based on the directed affinity evolution of inactivated carbohydrate-processing enzymes. As these reagents are derived from enzymes with very-high carbohydrate specificity, they do not suffer from the cross-reactivities frequently exhibited by both lectins and antibodies.

LECTENZ molecules are not limited to any specific carbohydrate processing enzyme. Rather, broadly applicable to any glycosidase or glycosyltrasferase enzyme, protein, or polypeptide capable of specifically recognizing a carbohydrate. Examples of glycosidases suitable for the present inventions include, but are not limited to, lactase, amylase, chitinase, sucrase, maltase, neuraminidase, invertase, hyaluronidase, and lysozyme. Glycosidases of the present invention can be inverting or retaining glycosidases. In one embodiment, a LECTENZ is prepared from PNGase F, isolated from Flavobacterium meningosepticum. In another embodiment, the lectenz is prepared from recombinant B-O-GlcNAcase, with the WT sequence as determined for β-O-GlcNAcase isolated from Bacteroides thetaiotaomicron. In yet another embodiment, neuraminidase from Clostridium perfringens is used to prepare a LECTENZ. In addition to glycosidases, carbohydrate-processing enzymes suitable for use in the present invention include glycosyltransfeases and polysacharide lyases. Other carbohydrate-processing enzymes include carbohydrate esterases, sulfatases, sulfotransferases, or any other enzyme that acts on a carbohydrate substrate. Catalytically inactive carbohydrate-processing enzymes of the present invention can be prepared from carbohydrate-processing enzymes isolated from prokaryotic or eukaryotic organisms, as well as others.

In certain embodiments, the carbohydrate-processing enzyme is a glycosidase enzyme. In other embodiments, the carbohydrate-processing enzyme is a glycosyltransferase enzyme. In other embodiments, the carbohydrate-processing enzyme is a polysaccharide lyase enzyme. In other embodiments, the carbohydrate-processing enzyme is a sulfatase enzyme. In other embodiments, the carbohydrate-processing enzyme is a sulfotransferase enzyme. In other embodiments, the carbohydrate-processing enzyme is a ligase enzyme. In further embodiments, the carbohydrate-processing enzyme is an amidase enzyme. In yet further embodiments, the carbohydrate-processing enzyme is an epimerase enzyme.

Representative carbohydrate-processing enzymes that can be used to form LECTENZ molecules suitable for use in the multiplexed assay of the invention include, without limitation, glycosidase enzymes, glycosyltransferase enzymes, polysaccharide lyase enzymes, sulfatase enzymes, sulfotransferase enzymes, ligase enzymes, amidase enzymes, and epimerase enzymes. Examples of LECTENZ molecules that make useful array elements include LECTENZ molecules derived from PNGase F (an amidase) and LECTENZ molecules derived from 0-GlcNAcase.

See WO2010/068817 (“Glycan-Specific Analytical Tools,” published Jun. 17, 2010), which is incorporated by reference herein in its entirety, for a more complete description of LECTENZ molecules.

A multiplexed suspension array according to the invention can be formed exclusively from lectins, antibodies or LECTENZ molecules; however it is expected that multiplexed arrays that incorporate multiple types of carbohydrate binding antibodies, such as both lectins and LECTENZ molecules, or both antibodies and lectins, or both antibodies and LECTENZ molecules, or all three types of carbohydrate binding molecules, with or without any other typed of carbohydrate binding molecules, will provide a more useful platform for glycoprofiling, as it will help to increase the certainty of identification of a particular glycan if one or more of the carbohydrate binding molecules that bind that glycan exhibit cross-reactivity with other glycans.

The MSA glycoprofiling approach of the present invention provides many advances and advantages over currently used technologies, including, MS, microplate assays, and solid phase microarrays. Some advantages include, but are not limited:

Storage and Handling is improved. Array elements have a long shelf life (>6 months at 4° C.), because the array elements are stored in buffer until use.

The addition on new elements is simplified. A suspension of microspheres typically contains tens of millions of particles per milliliter that, when coupled with the appropriate receptor can be used to prepare thousands of microsphere arrays. To reconfigure an array with new array elements, a new conjugation is performed on a particular microsphere subset and a new mixture of microspheres is prepared.

Array density is greatly increases. While the current generation of suspension arrays contain between a dozen and a hundred discrete array elements, optical encoding approaches make very high-density arrays possible.

Statistical reproducibility is improved. A few microliters of microspheres typically contain tens of thousands of array elements. Thus each element in the array is represented by several hundred individual microspheres, thus the flow-cytometric measurement represents a replicate analysis of each array element.

Throughput is increased. Using flow cytometry as a measurement platform, particle analysis rates can be as high as 10,000 s-1, making highly multiplexed analysis extremely rapid.

Ligand binding kinetics and thermodynamics are improved. The process is an equilibrium process, therefore making it possible to determine KA values. Liquid reaction kinetics gives faster, more reproducible results than with solid, planar arrays.

The approach is driven by increasing demand for analytical methods to measure large numbers of biomolecules quantitatively and sensitively in small volumes of sample.

Reduced cost and labor is obtained by multiplexing.

There is a shortened time to results by favorable reaction kinetics of liquid bead array approach, with smaller sample requirements.

A further advantage of the suspension array technology used with the present invention, both in terms of throughput and accuracy, is that, whereas procedures using flat microarrays often require extensive washing to reduce high background signals, the ability of flow cytometry to resolve free and bound probes enables assays to be performed with minimal or no wash steps, streamlining sample processing. In the particular case of glycoprofiling, it is notable that, lectins generally have low affinity for their carbohydrate ligands and the interactions may not be able to survive the extensive washing steps (Horimoto and Kawaoka, 2005, Nat Rev Microbial; 3(8):591-600).

The ability to perform multiplexed analyses of suspension arrays, in small sample volumes, for many target glycoprotein samples, makes the MSA glycoprofiling approach described herein a powerful alternative to less flexible flat surface arrays. By combining this technology with common and established cytometry instrumentation, there is a potential to make an almost immediate impact on the manner in which glycosylation analyses are performed. This approach should open the field of glycoprofiling up to laboratories that would otherwise find such analyses daunting, and should provide a tool to meet the unmet needs for real-time process control in the production of therapeutic glycoproteins.

Advantageously, the multiplexed suspension assay can include particles (array elements) with overlapping or redundant specificities, which can increase the level of confidence in the data obtained when analyzing or characterizing a carbohydrate containing sample.

It should be understood that the particular array elements used in the multiplexed suspension array technology are selected based upon the research or clinical interest of the user; indeed, the ability to formulate, in a modular fashion, a customized set of array elements is what imparts the unique flexibility to this technique. It is not possible to set forth herein every possible combination of array elements that might be of interest to a user nor should it be necessary, as one of skill in the art can readily imagine a vast number of permutations and can create a custom array of any number of array elements by functionalizing the desired number of beads with the desired number and type of carbohydrate binding molecules.

The present invention includes compositions and methods including any combination or subcombination of specific carbohydrate binding molecules described herein; for example, any two, any three, any four, any five, any six, any seven, any eight, any nine, any ten, any eleven, any twelve, any thirteen any fourteen, any fifteen, any sixteen, any seventeen, any eighteen, any nineteen, any twenty, or more of the a specific carbohydrate binding molecule described herein.

In some embodiments, the binding of the carbohydrates or carbohydrate containing compounds to the functionalized particles is conveniently detected or monitored using fluorescence-based techniques such as flow cytometry; however, other detection techniques are envisioned which may encompass both batch and flow process, and are selected based on the type of labeling agent used for the microspheres and/or the carbohydrate or carbohydrate containing compound (fluorescent, phosphorescent, magnetic, electromagnetic, radioactive, enzymatic, and the like). For example, any of the various detection methods and addressable particles reviewed in more detail in Braekmans et al., 2002, Drug Discovery; 1:447-456; Wilson et al., 2006, Agnew Chen Int Ed; 45:6104-6117; and Birtwell and Morgan, 2009, Integr Biol; 1:345-362 (which are herein incorporated by reference in their entireties) may be used.

Carbohydrates and carbohydrate containing compounds that can be detected using the multiplexed suspension assay of the invention include but are not limited to disaccharides, trisaccharides, oligosaccharides, polysaccharides, glycosides, glycans, glycosaminoglycans, glycoproteins, glycopeptides, glycolipids, glycoliopeptides, nucleotides, nucleosides and nucleic acids. A carbohydrate can include a monosaccharide, a disaccharide or a trisaccharide; it can include an oligosaccharide or a polysaccharide. An oligosaccharide is an oligomeric saccharide that contains two or more saccharides and is characterized by a well-defined structure. A well-defined structure is characterized by the particular identity, order, linkage positions (including branch points), and linkage stereochemistry (α, β) of the monomers, and as a result has a defined molecular weight and composition. An oligosaccharide typically contains about 2 to about 20 or more saccharide monomers. A polysaccharide, on the other hand, is a polymeric saccharide that does not have a well defined structure; the identity, order, linkage positions (including brand points) and/or linkage stereochemistry can vary from molecule to molecule. Polysaccharides typically contain a larger number of monomeric components than oligosaccharides and thus have higher molecular weights. The term “glycan” as used herein is inclusive of both oligosaccharides and polysaccharides, and includes both branched and unbranched polymers. When a carbohydrate contains three or more saccharide monomers, the carbohydrate can be a linear chain or it can be a branched chain.

Larger carbohydrate containing structures can also be detected using the multiplexed suspension assay of the invention. Examples of larger detectable structures include cell membrane components and cell wall components, components of an extracellular matrix, virions, virus particles, and partial or whole virus or partial or whole cells, including bacteria, yeast, protozoans and fungi.

Applications (and associated markets) of the glycoprofiling platform described herein include the characterization of isolated glycoproteins and the monitoring of glycosylation during glycoprotein expression.

Research groups and regulatory agencies need to characterize the glycoprofiles of specific, purified glycoproteins. The glycoprofiling platform described herein addresses this by providing insight into the relative levels of the terminal glycan components that define unique sequences associated with glycosylation. In addition, by careful choice of the carbohydrate-receptor proteins in the array, the linkages and configurations between the monosaccharides that comprise the glycans can be determined. This information will enable a researcher to elect whether or not to pursue more detailed analysis by MS. Moreover, when the carbohydrate-receptor protein is a reagent, such as a diagnostic antibody, the glycoprofiling platform described herein will be extremely useful in the screening of samples for the discovery of glycoproteins that carry disease marker glycans. The role of glycans in biological development and disease makes them obvious targets for detection, diagnostic, and therapeutic applications. A lack of sufficient glycan-specific analytical tools is responsible in part for the delay in fully exploiting aberrant glycosylation in the diagnosis and treatment of disease. There is an urgent need for biosensors with defined carbohydrate specificity that can be used to interrogate biological samples in the search for abnormal glycosylation. In a 2007 White Paper Report from Focus Groups at the NIH Workshop on Frontiers in Glycomics and Glycobiology, it was concluded that: “The analytical technology available for the specific analysis of glycoconjugates is lagging behind that of the technologies available to the scientific community for the study of genomics and proteomics and their function in disease and assigns the highest priority to the support of the development of glycan-specific analytical tools.”

From the perspective of a regulatory agency, or biopharmaceutical company, the glycoprofiling platform described herein provides a method for fingerprinting the glycosylation state, which would serve a key role in identifying batch variations in therapeutic glycoproteins. Such variations routinely occur, for example when a new cell-type is employed for expression, and may even arise from minor differences in growth medium.

Another major application for rapid glycoprofiling technologies is real-time monitoring of the glycosylation state of a protein during glycoprotein production. This need is unmet by existing technologies. An essential regulatory requirement in the commercial production of glycoproteins is maintaining uniform glycosylation profiles. Given that industrial fermentation scales may be up to 20,000 L per batch, post-production sample failure is an enormously costly event. The alternative industrial production mode, continuous flow, would equally benefit from real time glycoprofiling capability, particularly in that if variations in the glycoprofile were detected, the production stream could be diverted without contaminating the entire batch. Currently, it takes several weeks (months on occasion) to obtain protein quality data. As a result, it is difficult to efficiently incorporate these findings in routine process development

The multiplexed suspension assay described herein is especially useful in methods of glycoprofiling, including real-time analysis during synthesis of carbohydrate containing molecules, as described in more detail below. The multiplexed suspension assay described herein can provide complementary data to that from mass spectrometry (MS)-based methods. While not supplanting more precise techniques for final quality control, multiplexed suspension assay described provides a convenient method for monitoring glycosylation. Notably, the most sensitive methods, such MS are unable to directly determine the linkage type (1-2, 1-3, 1-4, etc.) or the anomeric configuration (α- or β-) between the monosaccharides in a glycan. Consequently, the glycoprofiles determined from MS methods always infer the glycan structure based on expected linkages and configurations. While this is adequate for certain portions of the glycan, which are invariant, it is inadequate for assigning the structures of variable regions. In particular, MS-based techniques cannot determine whether a sialylated glycan (a very common eukaryotic modification) terminates in a Sialylα2-3Gal or Sialylα2-6Gal linkage. Terminal sialylation is critical in determining the bioavailability of therapeutic glycoproteins Huang et al., 2006, Proc Natl Acad Sci USA; 103(1):15-20, can regulate protein function, particularly in the case of therapeutic antibodies (Wang et al., 2008, Proc Natl Acad Sci USA; 105(33):11661-11666; Werz et al., 2007, J Am Chem Soc; 129:2770-2771), can be a key virulence factor in pathogenic bacteria (Hakomori, 1984, Ann Rev Immunol; 2:103-26), and the difference between α2-6 and α2-3 linkages is responsible for defining whether pathogens, such as influenza, are transmissible between humans (α2-6) or not (α2-3).

The multiplexed suspension assay described herein can be used in a regulatory role to monitor batch consistency, as well as provide a routine tool for assessing protein glycosylation in a research environment. Providing the ability to rapidly monitor changes in the glycoprofile during glycoprotein expression would enhance the efficient production of commercial therapeutic glycoproteins.

The multiplexed suspension assays described herein have potential use as a method of detection in many areas, including environmental, fermentation, food and medical areas and could be used for in vivo or in vitro sensing in humans or animals. Environmental samples include, but are not limited to, air, agricultural, water and soil.

Glycans have several distinct properties that make them excellent targets for disease biomarkers. Firstly, the location of the glycans on the cell surface makes them the first point of contact of cellular interactions and thus crucial in the control of normal metabolic processes. Cell surface molecules are also strategically exposed for surveillance by the immune system allowing for the potential of immune recognition of abnormal cells. Secondly, specific glycan structures that are not present, or are in low amounts, in normal states proliferate in disease states. And lastly, changes in glycosylation involve many proteins, including those that are highly abundant. Therefore, a single change in a cell's glycosylation machinery can affect many different glycoconjugates.

In some embodiments, a multiplexed suspension assay as described herein can be used to interrogate biological samples in the search for abnormal glycosylation.

In other embodiments, a multiplexed suspension assay as described herein can be used for the detection of a target carbohydrate-based analyte level in biological fluids. Examples of the target analytes include, but are not limited to, endogenously found molecules, such as N- or O-linked glycans, glycosaminoglycans (including heparin), exogenously consumed species, such as plant polysaccharides, carbohydrate-based drugs, and pathogens, whose surfaces are often coated in complex distinct glycans.

Examples of biological samples include, but are not limited to, any biological fluid, tissue, or organ. Examples of the biological fluids include, but are not limited to blood, urine, serum, lymph, saliva, cerebra-spinal fluid, anal and vaginal secretions, perspiration and semen, of virtually any organism, with mammalian samples being preferred and human samples being particularly preferred.

In some specific embodiments, a multiplexed suspension assay as described herein can be used for diagnosing, and/or treating diseases manifested by abnormal glycosylation. Glycans can regulate different aspects of tumor progression, including proliferation, invasion and metastasis. Changes in glycosylation patterns have been observed in cancers including prostate cancer, colorectal cancer, and breast cancer. Glycoproteins have also provided an ideal source for discovering biomarkers for disease detection. A multiplexed suspension assay as described herein may be useful to identify potential biomarkers in cancer.

In other embodiments, a multiplexed suspension assay as described herein can be used in drug discovery and the evaluation of the biological activity of new glycan-based compounds.

The present invention includes kits including one or more of the compositions described herein, each composition having individually addressable particles; each individually addressable particle having an external surface and having linked to said external surface a separate carbohydrate binding molecule; and each individually addressable particle separately labeled with a detectable label. Each composition may be contained in a separate container or package. A kit may further include one or more secondary binding agents, with a binding specificity for an analyte. A kit may further include one or more reagents for directly labeling the analyte with a detectable label. A kit may further include packaging materials and/or instructions for use. A kit may further include positive and/or negative analyte controls. A kit may be formulated for research, industrial, medical, or veterinary use. A kit may be formulated for flow cytometry analysis. A kit may be formulated for image based analysis. A kit may further include one or more software components to assist in the calculation of relative glycan proportions in a sample.

A software component may assist, for example, in calculations glycan proportions, relative glycan compositions, and/or percentages of a given glycan determinant in a sample. In some embodiments, a software application as described herein is sold separately.

The present invention and/or one or more portions thereof may be implemented in hardware or software, or a combination of both. For example, the functions described herein may be designed in conformance with the principles set forth herein and implemented as one or more integrated circuits using a suitable processing technology, e.g., CMOS. As another example, the present invention may be implemented using one or more computer programs executing on programmable computers, such as computers that include, for example, processing capabilities, data storage (e.g., volatile and nonvolatile memory and/or storage elements), input devices, and output devices. Program code and/or logic described herein is applied to input data to perform functionality described herein and generate desired output information. The output information may be applied as an input to one or more other devices and/or processes, in a known fashion. Any program used to implement the present invention may be provided in a high level procedural and/or object orientated programming language to communicate with a computer system. Further, programs may be implemented in assembly or machine language. In any case, the language may be a compiled or interpreted language. Any such computer programs may preferably be stored on a storage media or device (e.g., ROM or magnetic disk) readable by a general or special purpose program, computer, or a processor apparatus for configuring and operating the computer when the storage media or device is read by the computer to perform the procedures described herein. The system may also be considered to be implemented as a computer readable storage medium, configured with a computer program, where the storage medium so configured causes the computer to operate in a specific and predefined manner to perform functions described herein.

The present invention and/or one or more portions thereof include circuitry that may include a computer system operable to execute software to provide for the determination of glycan composition. Although the circuitry may be implemented using software executable using a computer apparatus, other specialized hardware may also provide the functionality required to provide a user with information as to the physiological state of the individual. As such, the term circuitry as used herein includes specialized hardware in addition to or as an alternative to circuitry such as processors capable of executing various software processes. The computer system may be, for example, any fixed or mobile computer system, e.g., a personal computer or a minicomputer. The exact configuration of the computer system is not limiting and most any device capable of providing suitable computing capabilities may be used according to the present invention. Further, various peripheral devices, such as a computer display, a mouse, a keyboard, memory, a printer, etc., are contemplated to be used in combination with a processing apparatus in the computer system. In view of the above, it will be readily apparent that the functionality as described herein may be implemented in any manner as would be known to one skilled in the art.

The present invention is illustrated by the following examples. It is to be understood that the particular examples, materials, amounts, and procedures are to be interpreted broadly in accordance with the scope and spirit of the invention as set forth herein.

### EXAMPLES

### Example 1

### Target Enzymes for Conversion to Lectenz

### Target Enzymes for Conversion to Carbohydrate-Biosensors (Lectenz)

Presented in Table 1 are three initial glycosidases that can be subjected to redesign as lectenz. Lectenz 1 will find broad use in all aspects of glycomics analysis. Lectenz 2 will be vital to furthering the analysis of glycans in diabetes, and lectenz 3 will be useful in characterizing human versus avian influenza receptors.

TABLE 1
Initial target enzymes for conversion to carbohydrate-biosensors (Lectenz)

| | | | Source/ | |
|---|---|---|---|---|
| | | | Recombinant | |
| Lectenz | | | Expression | Available |
| ID | Enzyme | Specificity | Vector | Structure |
| 1 | PNGase F, Peptide-N4- | N-linked | F. meningosepticum/ | X-ray (b) |
| | (acetyl-β-D- | oligosaccharides (a)[ | E. coli | |
| | glucosaminyl)- | | | |
| | asparagine amidase | | | |
| 2 | β-O-GlcNAcase, N- | O-linked β-GlcNAc, | B. thetaiotaomicron/ | X-ray (d) |
| | acetyl-β-D- | monosaccharide (c) | E. coli | |
| | glucosaminidase | | | |
| 3 | Neuraminidase, N- | Terminal non- | C. perfringens/ | Comparative |
| | acetyl-neuraminate | branched α-(2,3) and | E. coli | model (f) |
| | glycohydrolase | α-(2,6)-Neu5Ac (e) | | |

Additional Targets

| | | | | |
|---|---|---|---|---|
| 4 | α-(1-2)-Mannosidase | α-(1-2)-Man on High | Human, | X-ray (g) |
| | | mannose N-glycans | mouse, S. cerevisiae/ | |
| | | | P. pastoris | |
| 5 | α-Galactosidase | α-Gal on | Human/ | X-ray (h) |
| | | glycoproteins and | human cells | |
| | | glycolipids | | |
| 6 | β-Galactosidase | β-Gal on | E. coli/E. coli | X-ray (i) |
| | | glycoproteins and | | |
| | | glycolipids | | |
| 7 | α-1,2/3/6-Mannosidase | α-1,2/3/6 Man on | Human, mouse/ | X-ray (j) |
| | | High mannose N- | P. pastoris | |
| | | glycans | | |

(a) Haslamet al., Core fucosylation of honeybee venom phospholipase A2. Glycobiology, 1994. 4(2): p. 105-6.
(b) Kuhn et al., Crystal-Structure of Peptide-N-4-(N-Acetyl-Beta-D-Glucosaminyl) Asparagine Amidase-F at 2.2-Angstrom Resolution. Biochemistry, 1994. 33(39): p. 11699-11706.
(c) Gao et al., Dynamic O-glycosylation of nuclear and cytosolic proteins: cloning and characterization of a neutral, cytosolic beta-N-acetylglucosaminidase from human brain. J Biol Chem, 2001. 276(13): p. 9838-45.
(d) Dennis et al., Structure and mechanism of a bacterial beta-glucosaminidase having O-GlcNAcase activity. Nat. Struct. Mol. Biol., 2006. 13(4): p. 365-71.
(e) Mizan et al., Cloning and characterization of sialidases with 2-6′ and 2-3′ sialyl lactose specificity from Pasteurella multocida. J. Bacteriol., 2000. 182(24): p. 6874-83.
(f) Pieper et al., MODBASE, a database of annotated comparative protein structure models, and associated resources. Nucleic Acids Res, 2004. 32(Database issue): p. D217-22.
(g) Tempel et al., Structure of Mouse Golgi a-Mannosidase IA Reveals the Molecular Basis for Substrate Specificity among Class 1 (Family 47 Glycosylhydrolase) a1,2-Mannosidases. J. Biol. Chem., 2004. 279(28): p. 29774-29786.
(h) Garman and Garboczi, The molecular defect leading to Fabry disease: structure of human alpha-galactosidase. J Mol Biol, 2004. 337(2): p. 319-35.
(i) Jacobson et al., Three-dimensional structure of beta-galactosidase from E. coli. Nature, 1994. 369(6483): p. 761-6.
(j) Heikinheimo et al., The structure of bovine lysosomal alpha-mannosidase suggests a novel mechanism for low-pH activation. J Mol Biol, 2003. 327(3): p. 631-44.

### Lectenz 1: Glycomics Affinity Reagent for Enrichment of N-Linked Glycans (Specific for N-Linked Oligosaccharides not Containing Core Fucose)

In many applications, particularly those that involve analysis of clinical tissue and fluids, there is great interest in identifying changes in protein and glycoprotein expression as a function of disease progression. If changes in the expression of specific molecules can be highly correlated with disease state, then they can be exploited to develop potential diagnostics and, perhaps, reagents to image diseased cells and tissues. A difficulty that underlies all of these analyses is because these mixtures are so complex, it is extremely challenging to develop separation methods that allow subsequent identification and quantification of the majority of individual species present in a sample using mass spectrometry. Furthermore, convincing evidence demonstrates that changes in the glycans expressed on proteins can serve as additional markers for disease. There is a great need, therefore, to be able to rapidly separate proteins from glycoproteins and peptides from glycopeptides to enable identification and quantification for correlation with disease states. In serum, for example, half of the components are estimated to be non-glycosylated, with albumin by far the most abundant. If these proteins could be separated from the glycoproteins, then the complexity falls by a factor of 2.

Moreover, glycomics sequencing is focused on the analysis of trypsin-produced glycopeptides in order to map particular N-linked glycan structures to each glycosylation site on each peptide. In this type of analysis, the majority of species produced by the proteolytic digest by far are non-glycosylated peptides. The ability to separate these from the glycopeptides of interest would greatly simplify the analysis to yield site-specific glycan information. The choice of PNGase F for the scaffold is based on the fact that this enzyme is widely used to remove N-glycans from glycoproteins and glycopeptides for further analysis. It is robust, and its action is universal, as long as peptides do not contain core α-1-3 fucosylation, which is not expressed in vertebrates. Further, it is fully active on species that express the α-1-6 fucosylation, such as vertebrates. The PNGase F lectenz would be extremely useful for separating glycoproteins and glycopeptides from extracts, fluids, and even purified glycoproteins for further detailed structural analysis by mass spectrometry. This reagent could be used to extract the needle (glycopeptides) from the haystack (proteolytic peptides) in a proteomics/glycomics analysis.

The initial PNGase lectenz scaffold will be generated from the enzyme produced by Flavobacterium meningosepticum, for which a crystal structure has been reported in complex with substrate. The presence of a crystal structure is significant for the computational optimization of lectenz-glycan affinity. In addition, preliminary point mutagenesis studies have identified a single mutation (D60N) that renders PNGase F completely inactive.

### Lectenz 2: Biosensor for Diabetes-Related Protein Hyper-GlcNacylation (Specific for β-O-GlcNAc)

A neutral, nucleocytoplasmic hexosaminidase was first described in 1975 and characterized as O-GlcNAcase in 1994. It has only recently been cloned (by Dr. L. Wells at the CCRC) and identified as a nucleocytoplasmic, neutral β-N-acetylglucosaminidase (O-GlcNAcase, OGA, EC 3.2.1.52). Unlike hexosaminidase A or B, OGA is localized to the cytosol and to a lesser degree the nucleus, has a neutral pH optimum, and does not catalyze the removal of nor is inhibited by GalNAc. The role of O-GlcNAcase in apoptosis has yet to be elucidated; however, cleavage of the enzyme near the middle of the polypeptide has no effect on enzyme activity, suggesting that the N-terminal “hyaluronidase-like” domain is sufficient for activity. Very recently crystal structures of O-GlcNAcase from Clostridium perfringens, both free and complexed with inhibitors, have been reported. They have high homology with the human protein and provide an excellent basis for computational studies.

### Lectenz 3: Biosensor for the Human Influenza Receptors α-2,6-Linked Neuraminic Acid (Specific for α-Neu5Ac-(2,6)-β-Gal)

The human cell-surface carbohydrate (neuraminic or sialic acid) targeted by the hemagglutinin protein of influenza virus. Hemagglutinin mediates attachment to and entry of the virus into host cells by binding to sialic acid receptors at the cell surface. Human influenza viruses preferentially bind to sialic acid linked to galactose by α-2,6 linkages; the main type found on the epithelial cells of the human upper respiratory tract. Avian viruses tend to bind to α-2,3 linkages that are found on avian intestinal epithelium and, to a lesser extent, the lower human respiratory tract. Thus, this receptor is a key determinant of host specificity and an important target for potential prevention and treatment of influenza. Our initial lectenz scaffold will be the 2,6-sialidase nanB, isolated from P. multocida, cloned and expressed in E. coli by colleagues at UGA (Dr. M. Lee, J Bacteriol. 2000 December; 182(24): 6874-6883.) This is so far the only neuraminidase that has a marked preference for the human 2-6 linkage. A 3D structure (Q27701) for this enzyme has been generated by comparative modeling, from PDB template 2SLI (a hydrolase in the same family) and deposited in the Modbase database, which will serve as a basis for the computational analysis. The ligand can be docked into the presumed active site using AutoDock with the GLYCAM parameters. Concurrently, characterization of the structure can occur experimentally using protein crystallography, NMR spectroscopy, and site-directed mutagenesis.

### MD Simulation of the PNGase F-Chitobiose Complex

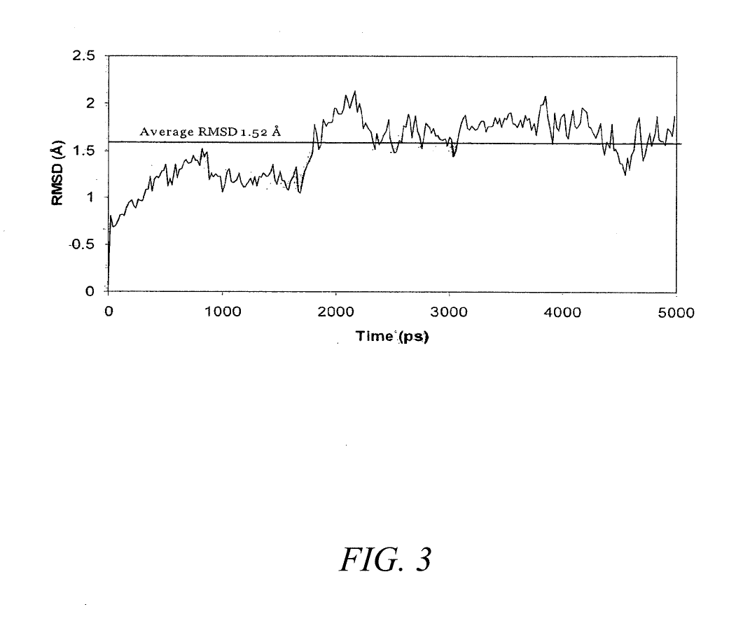

A 5 ns fully solvated MD simulation of the PNGase F-chitobiose complex was performed under nPT conditions (300 K, 1 atm) employing the AMBER/GLYCAM protein/carbohydrate force field and the experimental X-ray structure for the complex. Prior to energy analysis, the root mean squared difference (RMSD) in the positions of the Cα atoms was determined as a function of the simulation time (FIG. 3) to determine the stability of the simulation and the level of conformational equilibration. On the basis of that data, it was determined that the average RMSD (1.5 A) was reasonable; however, the simulation was slow to equilibrate. Consequently, the first 1 ns of data were not included in subsequent analyses.

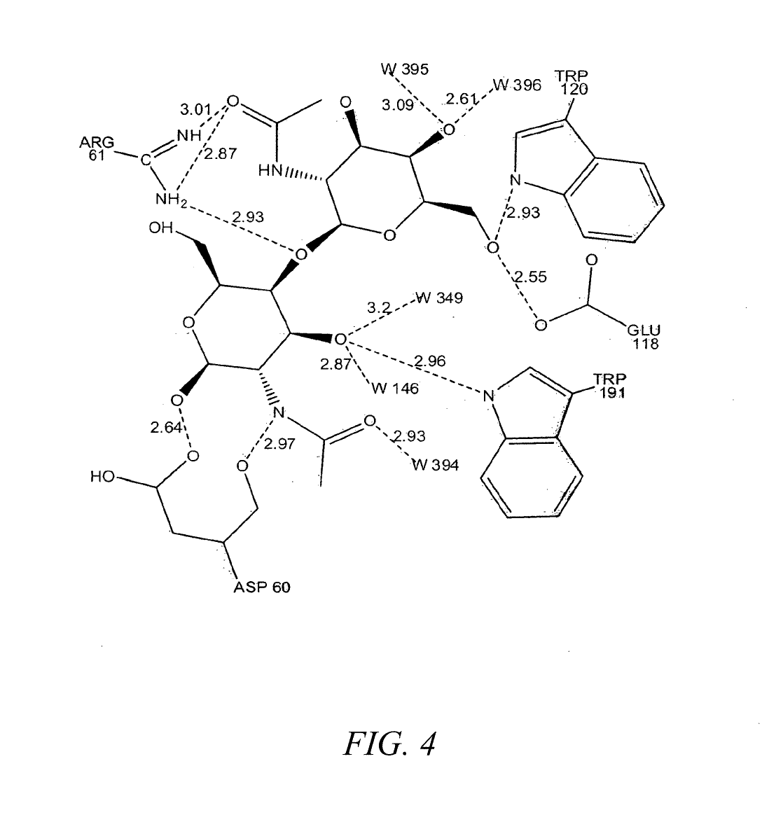

Ligand stability in the binding site was assessed by monitoring intermolecular hydrogen bonds between the chitobiose and the protein (FIG. 4). Average values for the hydrogen bonds and their percentage occupancies are presented in Table 2 along with the crystallographically determined values.

TABLE 2
Selected hydrogen bond lengths and occupancies
computed between chitobiose and PNGase F

| | | Average | |
|---|---|---|---|
| | | from MD | |
| Hydrogen bonds | X-ray(b) | Simulation | Occupancy |
| D60-Oδ - GlcNAc316 O1 | 3.02 | 2.76 ± 0.1 | 100% |
| D60-O - GlcNAc316 NAc | 2.97 | 2.84 ± 0.1 | 100% |
| R61-NH - GlcNAc317 OAc | 2.84 | 2.91 ± 0.1 | 93% |
| R61-NH - GlcNAc316-O4 | 2.92 | 2.90 ± 0.1 | 93% |
| R61-NH2 - GlcNAc317 OAc | 3.03 | 2.90 ± 0.1 | 76% |
| W120-Nε - GlcNAc317-O6 | 2.93 | 2.98 ± 0.1 | 39% |
| W191-Nε - GlcNAc316-O3 | 2.96 | 3.06 ± 0.1 | 10% |
| E118-Oε - GlcNAc317-O6 | 3.25 | —^{a} | |

^{a}Occupancies determined using a standard 3.3 Å cutoff.
(b)Kuhn et al., Crystal-Structure of Peptide-N-4-(N-Acetyl-Beta-D-Glucosaminyl) Asparagine Amidase-F at 2.2-Angstrom Resolution. Biochemistry, 1994. 33(39): p. 11699-11706.

Having confirmed that the MD simulation is stable and able to reproduce the experimental interactions between the ligand and the protein, it was then employed in subsequent binding energy and alanine scanning analyses.

### Identification of Hotspots and Key Residues by Interaction Energy Analysis

We employed the generalized Born (GB) continuum solvent model as implemented in AMBER, with solvent parameters developed for protein-ligand interactions. We have previously reported that these solvent parameters perform well for carbohydrate-antibody and carbohydrate-lectin MM-GB binding energy predictions. The contributions to the binding energy were computed for the 313 amino acids in PNGase F. The interaction energies are presented in Table 3 for all residues that are within 4.5 Å of the ligand (within a contact zone), as well as for any others that contributed at least less than −0.5 kcal/mol to either the total MM (van der Waals and electrostatic) interaction energy (ΔE_{MM}) or the total binding free energy (ΔG_{Binding}). Also included are any residues that contributed unfavorably to substrate binding, such as D60 and E206.

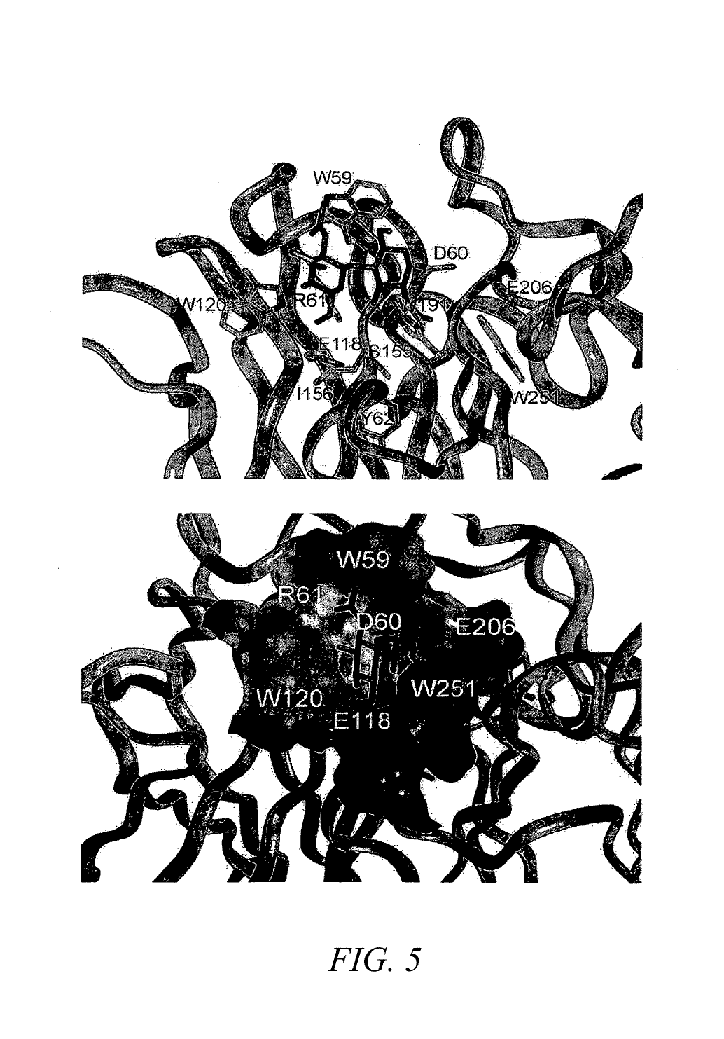

The total interaction energy (−13.7 kcal/mol) is comparable to data computed for the similar size galectin-1 LacNAc complex, and as in the case of galectin-1, overestimates the experimental affinity due to the omission of conformational and configurational entropy. Nevertheless, the per-residue interaction energies can be used to identify key amino acid residues. The majority of the per-residue net binding energies were favorable, with the notable exceptions of D60 and E206 (FIG. 5). D60 is the primary catalytic residue, while E206 and E118 are thought to be important for stabilization of reaction intermediates. Thus, it is significant, but perhaps not surprising, that these two residues were identified as the most destabilizing to the disaccharide product of the hydrolysis reaction.

A possibly unfavorable interaction was also exhibited by D57, which has an unfavorable ΔE_{MM} that is offset by a negative solvation free energy ΔG_{GB}. As has been observed for the galectin-1-LacNAc complex, the quasi-enthalpic contributions (ΔE_{MM}) and the entropy-related desolvation ΔG_{GB} terms are often similar in magnitude and opposite in sign, making the selection of the solvation model critical. In general, all such potentially key sites are included in the display library, rather than rely exclusively on the accuracy of the theoretical computations.

FIG. 5 represents, in the left image: residues within 4.5 Å of the disaccharide ligand (red) in the binding site of PNGase F. In the right image: the solvent accessible surface with the residues identified as most significant for binding labeled.

Direct comparisons with the data in Table 4 are possible with two similar carbohydrate-protein complexes (galectin-1—LacNAc and Con A—trimannoside), both of which employed the AMBER/GLYCAM force field and the GB solvation approximation. In Table 3, as in the MM-GB analysis of galectin-1 and Con A, the majority of the molecular mechanical energy (−33.8 kcal/mol) arises from electrostatic interactions (−20.8). However, both the van der Waals and the electrostatic contributions (−12.9 kcal/mol and −20.8 kcal/mol, respectively) are lower than those observed for related disaccharides bound to galectin-1 (−17 to −24 kcal/mol for van der Waals and −30.8 to −67.5 kcal/mol for electrostatics), strongly suggesting that there is room for affinity enhancement via side chain optimization in PNGase F. Further, it can be seen that electrostatic contribution is approximately cancelled by desolvation free energy. This phenomenon has been observed in both previous studies and can be a manifestation of entropy-enthalpy compensation. An advantage can therefore arise from cancellation of errors in the GB calculation.

On the basis of the energies in Table 3, the known inactive D60A mutant was generated and the energies recomputed. The binding energy markedly improved in the D60A mutation (total ΔE_{MM}=−35.5, ΔG_{GB}=19.6, and ΔG_{Binding}=−15.9, see Table 5) for a net gain in affinity of approximately 2 kcal/mol.

TABLE 3
Residue contributions (kcal/mol) to the binding free energy
for wild type PNGase F bound to substrate, chitobiose
(β-GlcNAc-(1,4)-β-GlcNAc-OH)

| Contact Zone Residues | ΔE_{VDW} | ΔE_{ELE} | ΔE_{MM} | ΔG_{GB} | ΔG_{Binding} |
|---|---|---|---|---|---|
| R61 | −1.5 | −15.1 | −16.7 | 12.3 | −4.4 |
| W120 | −3.1 | −2.3 | −5.4 | 1.9 | −3.5 |
| D60 (nucleophile in | −0.9 | −3.9 | −4.8 | 5.2 | 0.4 |
| enzyme) | | | | | |
| W59 | −3.1 | −0.2 | −3.3 | 0.3 | −3.0 |
| W191 | −1.3 | −1.6 | −2.9 | 1.3 | −1.6 |
| W251 | −0.7 | −0.3 | −1.0 | 0.1 | −0.9 |
| Y62 | −0.6 | −0.1 | −0.6 | 0.0 | −0.6 |
| E118 | −0.5 | −0.1 | −0.5 | 0.6 | 0.1 |
| I156 | −0.2 | 0.1 | −0.2 | −0.1 | −0.3 |
| S155 | −0.3 | 0.2 | −0.1 | −0.1 | −0.1 |
| G192 | 0.0 | 0.1 | 0.0 | 0.0 | 0.0 |
| E206 | −0.3 | 2.1 | 1.8 | −1.1 | 0.7 |
| Other potentially | | | | | |
| important residues | | | | | |
| identified from | | | | | |
| 313 total residues | | | | | |
| (\|ΔE_{MM}\| or | | | | | |
| \|ΔG_{Binding}\| ≥0.5) | | | ΔE_{MM} | ΔG_{GB} | E_{Total} |
| T119 | −0.2 | −0.6 | −0.7 | 0.8 | 0.1 |
| R248 | −0.1 | −1.2 | −1.4 | 1.2 | −0.1 |
| K123 | 0.0 | −0.5 | −0.5 | 0.6 | 0.1 |
| R125 | 0.0 | −0.4 | −0.4 | 0.6 | 0.1 |
| D57 | −0.1 | 3.0 | 2.9 | −3.5 | −0.6 |
| Total Binding Energy | −12.9 | −20.8 | −33.8 | 20.1 | −13.6 |
| ΔG_{Binding} | | | | | |

TABLE 4
Total interaction energies (kcal/mol) for favorable mutants identified
by alanine and electrostatic scanning of PNGase F bound to chitobiose

| Contact Zone Residues | ΔE_{MM} | ΔG_{GB} | ΔG_{Binding} |
|---|---|---|---|
| Wild type enzyme (D60) | −37.1 | 23.5 | −13.7 |
| D60A | −35.5 | 19.6 | −15.9 |
| E206A | −40.7 | 23.6 | −17.2 |
| D60A/E206A | −37.2 | 21.0 | −16.4 |

Subsequently, alanine scanning was performed on the D60A mutant to look for possible further key residues (Table 5). One double mutant was subsequently identified (D60A/E206A) with any enhanced affinity (−0.5 kcal/mol), while five residues were confirmed as being critical to ligand binding (W251, W191, W120, W59, and R61).

TABLE 5
Relative^{a} interaction energies (kcal/mol) predicted from
alanine scanning for the D60N mutant of PNGase F bound to chitobiose

| Mutation | ΔΔE_{MM} | ΔΔG_{GB} | ΔΔG_{Binding} |
|---|---|---|---|
| D60A | 1.8 | −4.0 | −2.2 |
| E206A | −1.9 | 1.2 | −0.7 |
| D60A/E206A^{b} | −1.9 | 1.4 | −0.5 |
| D60A/S155A | −0.1 | 0.0 | −0.1 |
| D60A/I82A | 0.3 | 0.1 | 0.4 |
| D60A/Y62A | 0.1 | 0.0 | 0.1 |
| D60A/I156A | 0.1 | 0.1 | 0.2 |
| D60A/E118A | −0.2 | 0.2 | 0.0 |
| D60A/W251A | 1.0 | 0.0 | 1.0 |
| D60A/W191A | 2.6 | −1.0 | 1.5 |
| D60A/W59A | 3.0 | −0.4 | 2.6 |
| D60A/W120A | 5.1 | −2.1 | 3.0 |
| D60A/R61A | 15.5 | −11.7 | 3.8 |

^{a}Relative to wild type sequence (D60).
^{b}Double mutants are relative to initial mutant (D60A).

Rather than performing side chain repacking experiments initially, scanning the inactive mutant for positions that could lead to favorable electrostatic interactions was performed. This scanning was performed with both theoretical positive and negative probe residues (see experimental design) over all of the residues in the immediate contact zone (Table 6).

Although no mutations to charged residues were predicted to lead to enhanced total binding energies, several possible mutations were suggested to lead to improved molecular mechanical interactions. Thus, residues E206, 5155, E118, and Y62 can each be mutated to Arg and Lys and the energies recomputed. The resulting binding free energies can be used to select any further specific point mutants to clone and over-express. This perhaps is not surprising given that carbohydrate-protein interactions are characterized by an intricate network of hydrogen bonds, and perturbations of that network might rarely be favorable. It is again significant that E206 and E118, which have both been implicated in the enzyme mechanism, have been identified as potential key residues for affinity optimization. It is also notable for the design of the display library that no mutations to negatively charged residues were predicted to lead to improved affinities.

TABLE 6
Interaction energies relative to D60A mutant for key residues
predicted from electrostatic alanine scanning.

| Contact | | ALA⁺ | |
|---|---|---|---|
| Zone Residue | ΔΔE_{MM} | ΔΔG_{GB} | ΔΔG_{Binding} |
| E206A⁺ | −3.5 | 3.7 | 0.2 |
| S155A⁺ | −1.0 | 2.7 | 1.6 |
| E118A⁺ | −0.7 | 2.9 | 2.1 |
| Y62A⁺ | −0.4 | 4.2 | 3.8 |

Based on the computational affinity data, several mutants were selected for cloning and have been over-expressed in E. coli. The results of experimental affinity analyses are presented in the following section.

### Experimental Binding Affinity Measurements for PNGase F Lectenz

Presented in Table 7 are the dissociation constants measured using surface Plasmon resonance (SPR) for the interaction between denatured RNase B, which contains a single N-glycosylation site predominantly occupied by high mannose oligosaccharides and mutants of PNGase F.

TABLE 7
Dissociation constants measured for the interaction between denatured
glycoprotein RNase B and lectenz mutants of PNGase F.

| | | Relative^{c} | | Relative^{c} | |
|---|---|---|---|---|---|
| Lectenz | K_{d} | Enhancement | Lectenz | Enhancement | K_{d} |
| D60 (wild type)^{a} | 6.4 × 10⁻³ | 1 | D60A/E206K | 360 | 1.8 × 10⁻⁵ |
| D60A | 1.1 × 10⁻⁵ | 580 | D60A/R125A | 360 | 1.8 × 10⁻⁵ |
| D60N^{b} | 2.1 × 10⁻⁵ | 290 | D60A/E206R | 240 | 2.7 × 10⁻⁵ |
| E206A | 1.1 × 10⁻⁵ | 580 | D60A/E206Q | 360 | 1.8 × 10⁻⁵ |
| D60A/E206A | 2.0 × 10⁻⁵ | 320 | D60A/D57A | 910 | 7.0 × 10⁻⁶ |

^{a}Kₘ.
^{b}Reported inactive mutant.
^{c}Relative to wild type.

As predicted computationally, both the E206A and D60A mutants have markedly enhanced binding. Also, as suggested from electrostatic scanning, a positive charge (K or R) at E206 provides a modest further increase in affinity. At present, without the benefit of side chain repacking experiments or saturation mutagenesis, the first generation lectenz has micromolar affinity and with only two point mutations, has reached the micromolar level. It is worth noting that these preliminary mutations have enhanced the affinity of the PNGase lectenz nearly to that exhibited by the lectin Con A for high mannose oligosaccharides (K_{d}≈1×10⁻⁶M).

As mentioned in section B, high affinity is only one desirable property for a biosensor. High affinity will permit the reagent to be employed in affinity chromatography. However, it is also important to achieve a slow off-rate (k_{off}) if the biosensor is to be used successfully in such applications as tissue staining.

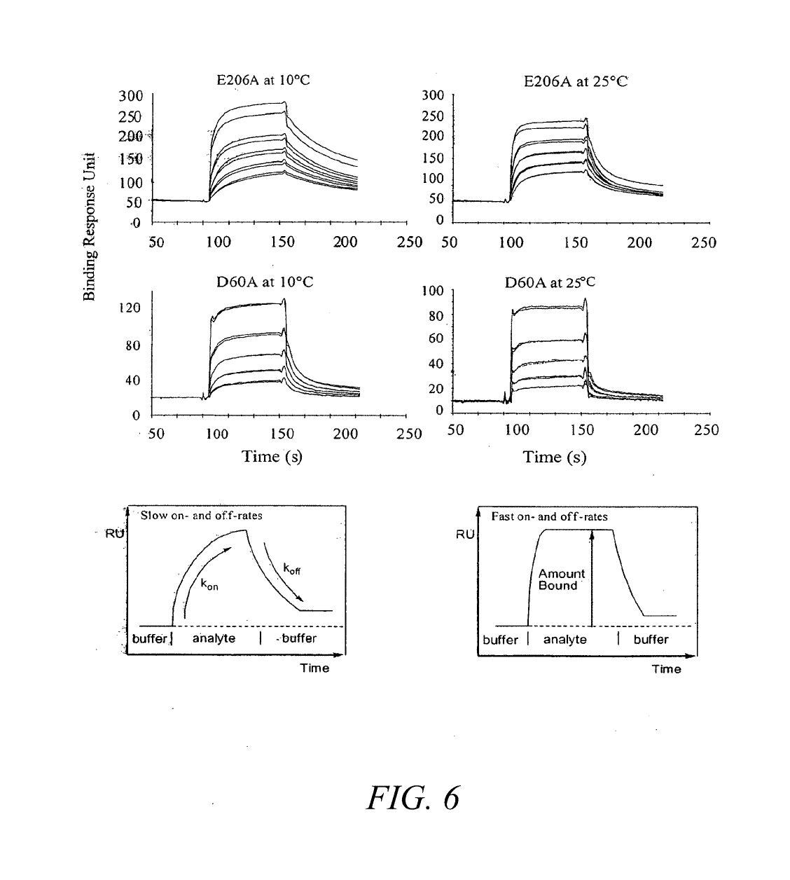

SPR provides a convenient method for assessing variations in k_{off}. An examination of FIG. 6 indicates significantly different kinetic behavior between mutants D60A and E206A. Both mutants have similar values for K_{d} (Table 7), but D60A displays rapid on and off rates (at both 10 and 25° C.), while E206A presents substantially decreased off-rates at both temperatures. This is an extremely significant feature as it suggests that the kinetics of binding will be tunable to achieve a range of properties.

### Further Affinity Enhancement

On the basis of the computational data, a focused yeast display library for the inactive D60A mutant containing the following 7 residues: D57, Y62, E118, S155, 1156, G192, and E206 was developed. These 7 positions were randomized to all 20 amino acids, resulting in a theoretical diversity of 20⁷ (≈10⁹) clones at the amino acid level.

In addition, computational side chain repacking experiments on a subset of the same residues Y62, E118, 1156, S155, G192, E206, and D57 employing the D60A mutant can be performed. By performing computational mutagenesis on the same set of key residues, we expect to be able to discover the extent to which the computational analysis is able to reproduce the optimized mutagenesis data. The effects of modifications of the computational method (such as implicit solvation model) so as to enhance its accuracy can be performed.

### Example 2

### Directed Evolution of Lectenz

A DNA library was created based on the inactive D60A mutant of the PNGase F enzyme. The residues D57, Y62, E118, S155, I156, G192, and E206 identified from computational analysis were randomized at the DNA level to encode for all twenty amino acids. The library was cloned into the yeast display vector pPNL6 and transformed into yeast.

The library was panned against dRNAse B captured on magnetic beads for two rounds then sorted for c-myc positive yeast by flow cytometry in the third round. The three rounds were repeated once for a total of six rounds. Table 8 shows the enrichment of yeast clones by sequencing the DNA of 18 clones from round six.

TABLE 8
Enrichment of clones from round six.

| | Clone |
|---|---|
| Round 6 Clones | Enrichment |
| R6.1.7 | 3/18 |
| R6.1.12 | 4/18 |
| R6.1.13 | 3/18 |

Clone R6.1.13 was selected for functional analysis using a competition assay and was expressed in bacteria and purified. In the assay, 50 μL of a 1 μM solution of R6.1.13 was preincubated with dRNAse B beads. Similarly, 50 μL of a 1 μM solution of the inactive enzyme D60A mutant was preincubated with dRNAse B beads. To each pre-incubated solution, Con A lectin (fluorescently labeled with DyLight 488) was added to a final concentration of 100 nM. Labeled Con A was also added to beads with and without dRNAse B as controls to a final concentration of 100 nM. The fluorescence of the beads was measured by flow cytometry. The fraction of Con A bound was normalized to the fluorescence of beads with and without dRNAse B (see FIG. 9).

Clone R6.1.13 protein showed approximately a 36% increased inhibition of Con A binding to dRNase beads, compared to the inactive enzyme D60A mutant, indicating affinity enhancement. This clone has not been fully optimized, as indicated by the modest clone enrichment of 3/18 (Table 8), and so further affinity improvements can be obtained by further rounds of enrichment.

### Example 3

### Preparation of Beads

Multiplex beads were purchased from Spherotech (Lake Forest, Ill.). Lectins were purchased from Vector Labs and EYLabs and conjugated to the beads using standard coupling chemistry with EDC (1-ethyl-3-[3-dimethylaminopropyl] carbodimide hydrochloride) and Sulfo-NHS (N-hydroxysulfosuccinimide). Glycans will be obtained from commercially available sources. In a typical assay, 200 nM carbohydrate solutions are preincubated with 50 nM SA-Alexa Fluor 488 for 30 minutes in 50 μL total volume. 20,000 of each bead is added and incubated for 30 minutes. The beads are then washed and fluorescence intensity measured by flow cytometry. Binding analyses will be performed as described previously (Nolan et al., 2006, Curr Protoc Cytom; Chapter 13: Unit 13.8; Yang and Nolan, 2007, Cytometry A; 71(8):625-31; Nolan and Yang, 2007, Brief Funct Genomic Proteomic; 6(2):81-90).

For standardization of bead preparations the performance of each batch of MSA reagents will be confirmed by using reference glycans, such as those presented in Table 9. The minimum signal/noise (S/N) ratio that permits reliable identification of the binding event will be determined and employed as a lower limit for MSA batch acceptability.

TABLE 9
Array elements and associated reference glycan.

| MSA Reagent | Biotinylated glycans |
|---|---|
| Sambucus nigra lectin I (SNA-I) | Neu5Acα2-6[Galβ1- |
| Polyporus squamosus (PSL) | 4GlcNAc β1-3]₂β- |
| Maackia amurensis lectin II (MAL II) | Neu5Acα2-3[Galβ1- |
| Maackia amurensis lectin (MAA) | 4GlcNAc β1-3]₂β- |
| Griffonia simplicifolia lectin II (GS II) | GlcNAcβ- |
| Conconavalin A (ConA) | Manα- |
| Erythrina cristagalli lectin (ECA) | [Galβ1-4GlcNAc β1-3]₂β- |
| Cholera toxin B subunit (CTB) | Neu5Acα2-3[Galβ1- |
| | 3GlcNAcβ1-4]Galβ1-4Glcβ- |

To demonstrate the ability of the glycoprofiling MSA approach to quantify glycan binding affinity in terms of equilibrium binding constants, titration of the standard glycans will be performed in the Glycoprofiling Multiplex Suspension Array to generate binding curves from which apparent dissociation constants for the glycans will be determined.

### Example 4

### Glycoprofiling with Multiplexed Suspension Arrays to Distinguish Between Two Glycosylation Sequences

This example assessed the performance of the Glycoprofiling Multiplexed Suspension Array (MSA) with standardized samples of glycans. A multiplexed suspension array (MSA) was prepared by conjugating a subset of five lectins (See Table 10) with known specificities to multiplex microspheres (FIG. 13). Glycans with known structures were obtained from the CFG and assayed for binding to the MSA lectins employing flow cytometry. Unconjugated microspheres or microsphere conjugated to a nonspecific protein may also be used as negative controls.

TABLE 10
Carbohydrate-specific reagents

| Microsphere | MSA Reagent | Specificity |
|---|---|---|
| 1 | Ethanolamine quenched | Negative control |
| 2 | Sambucus nigra lectin I (SNA-I) | Neu5Acα2-6Gal |
| 3 | Maackia amurensis lectin II (MAL II) | Neu5Acα2-3Gal |
| 4 | Griffonia simplicifolia lectin II (GS II) | Terminal GlcNAc |
| 5 | Concanavalin A (ConA) | Terminal Man |
| 6 | Erythrina cristagalli lectin (ECA) | Galβ1-4GlcNAc |

The ability of the MSA glycoprofiling arrays to distinguish between two glycosylation sequences was compared, and, in addition, both direct and secondary detection methods were tested (Table 11).

TABLE 11

| Glycans | | | | |
|---|---|---|---|---|
| Glycan | Glycan Analyte | Spacer | Fluorophore | Role |
| 1 | GlcNAcβ1-4GlcNAcβ-Sp- | Multivalent | Fluorescein | Positive Control |
| | | NHCOCH₂NH | | |
| | | PAA | | |
| 2 | GlcNAcβ1-4GlcNAcβ-Sp-Biotin | Multivalent | Streptavidin-Alexa | Positive Control |
| | | NHCOCH₂NH | Fluor 488 | |
| | | PAA | | |
| 3 | Neu5Acα2-6[Galβ1-4GlcNAcβ1- | Monovalent | Streptavidin-Alexa | Positive Control |
| | 3]2β-Sp-Biotin, also known as 6′S- | Sp-NH-LC-LC | Fluor 488 | |
| | Di-LN | | | |
| 4 | Neu5Acα2-3[Galβ1-3GlcNAcβ1- | Monovalent | Streptavidin-Alexa | Positive Control |
| | 4]Galβ1-4Glcβ-Sp-Biotin, also | Sp-NH-LC-LC | Fluor 488 | |
| | known as GM1 | | | |

Microspheres exist with sufficient fluorescence dynamic range to permit the routine multiplexed analysis of up to approximately 100 unique elements. Illustrated in FIG. 13 is a typical data set from the multiplexed cytometric analysis of the six component MSA Glycoprofiling assay, showing the free and bound bead states.

Direct detection of PAA-conjugates (a model for the analysis of directly-labeled high avidity glycoproteins). GlcNAcβ1-4GlcNAcβ-PAA-fluorescein (Table 11, Glycan 1) is a synthetic polymer, in which the carbohydrate is displayed in a multivalent format that is similar to a high-avidity biological context. The amide groups of the polymer chain were N-substituted with the sugar in a 4:1 ratio, and with fluorescein in a ratio of 100:1. By virtue of it being chemically conjugated to fluorescein, beads that bind to this polymer may be directly detected in the cytometer. Direct labeling could similarly be employed for the analysis of purified glycoprotein samples, but might not be suitable for in-process monitoring, in which the laborious step of isolation and purification should be avoided.

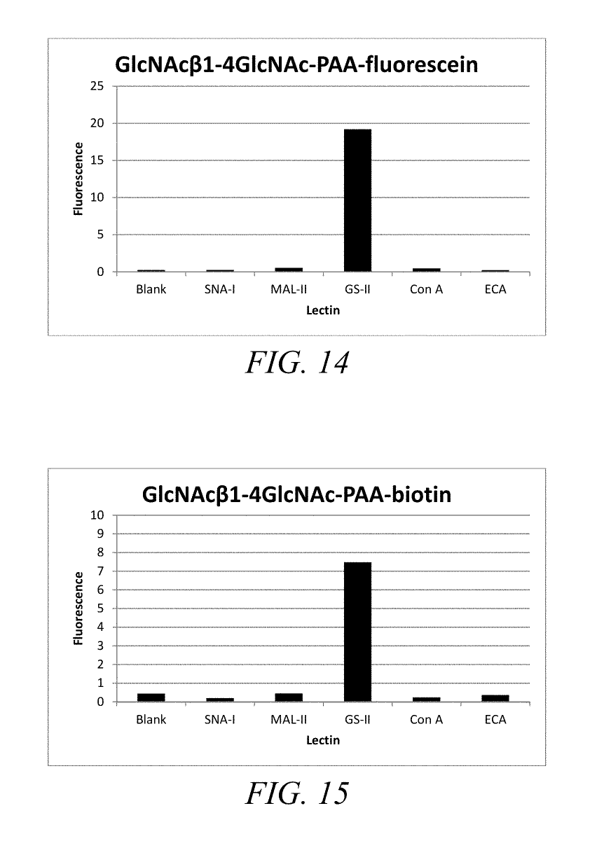

As seen in FIG. 14, the multiplexed analysis gave an excellent signal to noise ratio (S/N >20:1) for all of the detected elements. The GlcNAcβ1-4GlcNAcβ-PAA-conjugate bound to the MSA bead conjugated to GS II, which is a lectin specific for terminal GlcNAc. None of the other MSA elements, including the negative control bound to this glycan. Due to the relatively high concentration of glycans, the PAA conjugates represent a biological context that might be present for example on a mammalian or bacterial cell surface.

Secondary detection of PAA-conjugates (a model for the analysis of unlabeled high avidity glycoproteins). GlcNAcβ1-4GlcNAcβ-PAA-biotin (Table 11, Glycan 2) is also a synthetic polymer. As in the PAA-fluorescein system, the amide groups of the polymer chain were N-substituted with the sugar in a 4:1 ratio, although with biotin in a ratio of 20:1. In contrast to the case of PAA-fluorescein, the biotinylated polymer is used together with a streptavidin Alexa Fluor 488 conjugate for detection. The biotinylated carbohydrate polymer was preincubated with streptavidin-Alexa Fluor 488 in a 4:1 ratio and subjected to analysis (FIG. 15).

A secondary detection step was employed to mimic the application to unlabeled glycoproteins, as in the application of in-process glycoprofile monitoring. In the more general bioprocess case, secondary detection would be performed with an antibody specific for the target glycoprotein. If such an antibody were not be available, direct labeling would be an alternative. However in the commercial production of recombinant glycoproteins, specific antibodies are routinely employed for characterization.

As in the case of direct detection, MSA Glycoprofiling employing secondary detection with labeled-streptavidin correctly identified the glycan as terminating in GlcNAc. It is notable that the signal to noise was again excellent (S/N >10:1). Based on the PAA studies, either direct or secondary detection methods appear to be effective.

Secondary detection of biotinylated glycans (a model for the analysis of unlabeled low abundance glycoproteins). Unlike the PAA-conjugates, most glycoproteins will have lower levels of glycosylation, for example the therapeutic glycoprotein erythropoietin has three N-linked and one O-linked glycosylation positions. Terminal sialylation is critical to the activity and serum half life of therapeutic recombinant glycoproteins, such as human erythropoietin (EPO; the 3D structure of EPO can be found, for example, on the World Wide Web at glycam.org), and so we selected a glycan (Table 11, Glycan 3) that contained a terminal Neu5Acα2-6Gal sequence for analysis.

In addition, in order to assess the performance of the MSA glycoprofiling assay with glycans in a low avidity interaction typical of this type of glycoprotein, the use of the PAA polymer was eliminated. Instead, the monomeric-biotinylated glycan (the SpLCLC spacer is monomeric) was employed. And to mimic the case of bioprocess glycoprofiling, the streptavidin secondary detection system was retained.

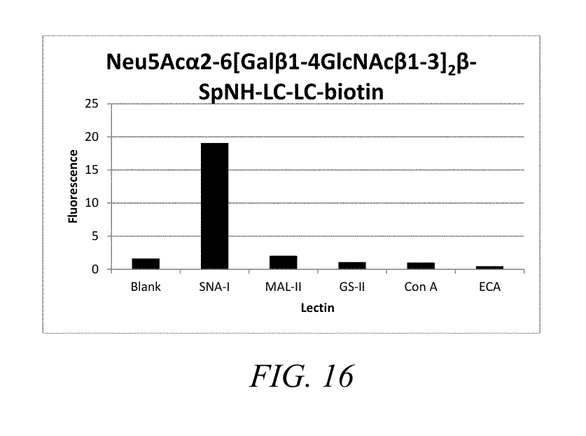

The results for Glycan 3 (FIG. 16) indicate that 6'S-Di-LN bound specifically to MSA bead SNA I, which is specific for Neu5Acα2-6Gal (Table 10). Negligible binding to any of the other MSA elements, including the ethanolamine quenched (blank) control beads, was seen. The signal to noise was again in the range of S/N 10:1.

### Example 5

### Multiplexed Suspension Array

### Materials

Activation buffer: 0.1 MES, 0.5 M NaCl, pH. 6.0
Coupling buffer: 0.1 M Sodium phosphate, 0.15 M NaCl, pH 7.4
Wash buffer: PBS/0.02% Tween20

### Ice Bucket, Ice

SPHERO™ Carboxyl Flow Cytometry Multiplex Bead Assay Particles (1×10⁸/ml)
Lectin (1 mg/ml)
EDC (1-ethyl-3-[3-dimethylaminopropyl]carbodimide hydrochloride) (191.7 g/mol)
Sulfo-NHS (N-hydroxysulfosuccinimide) (217.14 g/mol)

### Carbohydrates:

100 μM GM1-biotin

100 μM 3'S-Di-LN-LC-LC-biotin (2,3)

100 μM 6'S-Di-LN-LC-LC-biotin (2,6)

### Lectin Solutions

SNA-I (2 mg) was resuspended in 2 mL of solution having 0.01 M phosphate, 0.15M NaCl, and 0.05% sodium azide at pH7.4.

GS-II (1 mg) was resuspended in 1 mL of solution having 0.01 M phosphate, 0.15M NaCl, 0.5 mM CaCl₂, and 0.05% sodium azide at pH 7.4.

### Conjugation of Protein to Microsphere

In brief, the bead conjugation was performed using standard EDC/NHS chemistry. Alternatively, the carboxyl groups of proteins can be conjugated to amino microspheres using the same chemistry.

100 μl of microspheres in PBS were placed in a microfuge tube with 325 μL of activation buffer. EDC was dissolved at 100 mg/ml, 522 mM (20 mg in 0.2 mL) in activation buffer. Sulfo-NHS was dissolved at 100 mg/ml, 460 mM (20 mg in 0.2 mL) in activation buffer. 20 μl EDC and 55 μl Sulfo-NHS were then added to each tube. The tubes were incubated for 15 minutes at room temperature.

Following the incubation, the tubes were washed with 1× coupling buffer by spinning at 10000×g for 5 minutes then removing supernatant.

0.1 mL (100 μg) of lectin was added to each tube and 0.4 mL coupling buffer was added to each tube. The tubes were incubated 1 hour at 4° C. with mixing.

The tubes were washed 2× with wash buffer as described above. The remaining pellet was resuspended in 500 μL (2×10⁷/mL) PBS.

### Binding of Carbohydrates to the Protein-Conjugated Microspheres

In brief, biotinylated glycans were incubated with the lectin-conjugated beads, washed, and then detected by a fluorophore labeled streptavidin. Alternatively, the biotinylated glycans can be preincubated with the streptavidin-fluorophore conjugate in a 4:1 ratio.

Standard glycoproteins were biotinylated and measured the same way. Directly labeled glycoproteins or fluorescent antibodies against glycoproteins can also be used.

1 μL of each bead was mixed together. A total bead mixture equivalent to 1 μL of each type of beads were added to 50 μL 50 nM biotinylated carbohydrate. Samples were incubated for 1 hour with occasional vortexing. The supernatant was removed. Beads were washed 1× with 0.5 mL buffer by spinning at 3000 rcf/3 minutes/25° C. and removing supernatant. The beads were resuspended in 50 μL Streptavidin-Phycoerythrin (SA-PE) (1:200 dilution of a 1 mg/1 ml solution) in PBS. In later experiments SA-Alexa Fluor 488 was used. Samples were incubated for 1 hour with occasional vortexing. The supernatant was removed. The beads were washed 1× with 0.5 mL buffer by spinning at 3000 rcf/3 minutes/25° C. and removing supernatant. Beads were resuspended in 200 μL PBS buffer immediately before flow cytometry.

### Results

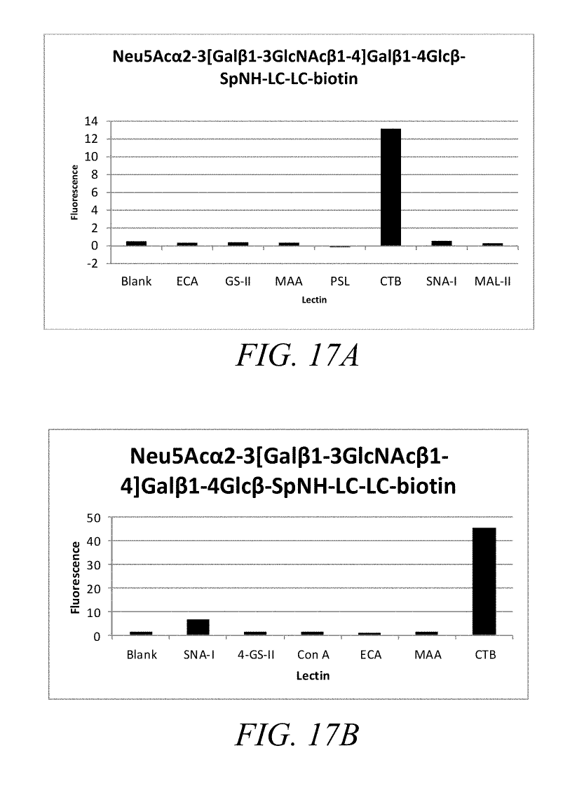

Specificity of the reagents in a multiplexed glycoprofiling suspension array is indicated by the data in FIGS. 17A and 17B, which shows that a glycan containing Neu5Acα2-3[Galβ1-3GlcNAβ1-4]Galβ1-4Glcβ-(GM1) bound only to the protein cholera toxin B subunit (CTB). CTB is known to be specific for glycans containing GM1. None of the other proteins included in this initial array bound to the GM1 glycan.

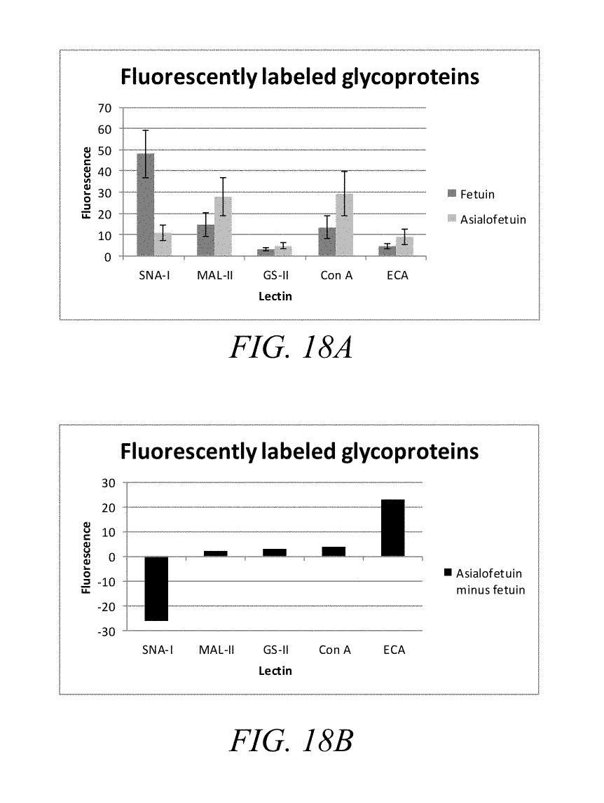

The ability of the reagents to detect glycosylation in glycoproteins (fetuin and asialofetuin) is demonstrated in FIGS. 18A and 18B, which demonstrates that treatment of the glycoprotein (fetuin) with neuraminidase (also known as sialidase) results in the formation of asialofetuin. Treatment with sialidase decreases the amount of sialic acid (also known as Neu5Ac) present in the glycoprotein, revealing terminal galactose. The loss of terminal sialic acid upon treatment of fetuin with sialidase is indicated by the decrease in the binding signal from the protein SNA I, which is specific for glycans containing terminal 2,6 linked sialic acid. The resulting exposure of terminal galactose is indicated by an increase in the binding signal from the protein ECA, which is specific for terminal galactose.

### Example 6

### Glycoprofiling

Glycoprofiling of Isolated Glycoproteins. MESF (Molecules of Equivalent Soluble Fluorochrome) microspheres quantitate the level of glycosylation in microspheres. Commercially available microspheres (for example, purchased from Bangs Labs; Fishers, Ind.) may be used. The MESF value of a bead equals the fluorescence intensity of a given number of pure fluorochrome molecules in solution. For example, an Alexa Fluor 488 microsphere with an MESF value of 10,000 has the same fluorescence intensity as a solution containing 10,000 Alexa Fluor 488 molecules. An MESF kit contains a set of microspheres with discrete levels of fluorochrome. By plotting each population's fluorescence intensity versus the MESF, a standard curve is generated. Such a relationship enables the linearity of the instrument to be confirmed, and the MESF value of the MSA bead can be extrapolated based on this standard curve. Using the MESF value of the MSA bead and the degree of labeling of the glycoprotein, the absolute number of glycoprotein molecules bound to each MSA bead can be determined.

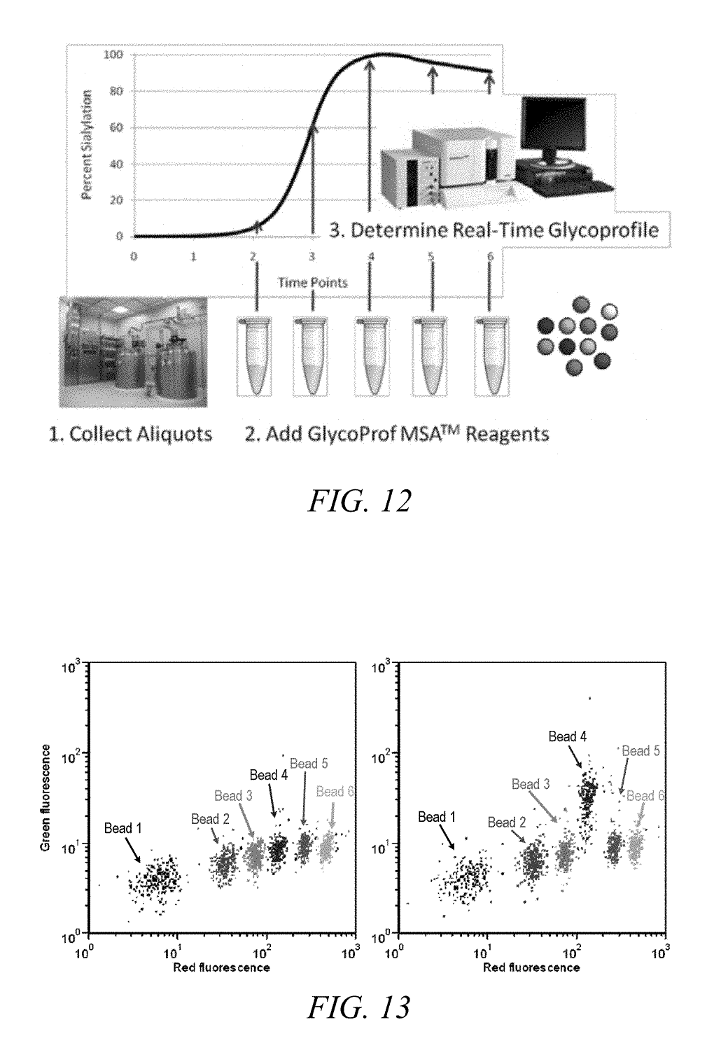

Glycoprofiling during Glycoprotein Expression. The process for in-process glycoprofiling is presented in FIG. 12. In order to maximize the turnaround time for this application, a secondary reagent, such as a labeled antibody or antibody fragment that is specific for the target glycoprotein, is employed for detection, eliminating the need to isolate and purify the expressed glycoprotein. N-Glycanase-PLUS (Prozyme) will be used to deglycosylate the glycoprotein specific antibody, prior to employing it in the assay to avoid interference. As it is not necessary to quantify the glycoprotein levels in order to determine the point at which the glycosylation profile reaches optimal levels, the use of a calibration curve, while possible, is not required.

### Example 7

### Confirmation

The accuracy of the glycoprofiles determined using the Glycoprofiling Multiplex Suspension Array method described herein will be confirmed by assaying glycoprotein samples whose glycoprofiles have already been determined or will be determined independently by complementary methods. Further, the glycoprofiles of biomedically relevant glycoproteins will be determined.

### Example 8

### Glycosidase Treatment

The Glycoprofiling Multiplexed Suspension Array described herein will be used to assay the effect on glycoprotein glycosylation profiles arising from glycosidase treatment with at least three glycosidases. Glycoprotein standards will be treated with glycosidases to generate altered glycosylation states, enabling an assessment of the sensitivity and accuracy of the Glycoprofiling Multiplexed Suspension Array when applied to glycoprotein samples. The necessary glycosidases are readily available and are routinely employed for glycan re-modeling. The glycosidases may be employed sequentially, for example to remove any terminal sialic acid, then to remove the subsequently-exposed Gal residues, then to remove the subsequently-exposed GlcNAc, etc. These will be applied to commercially available glycoproteins, such as RNase B, fetuin, sialoglycoprotein, glycophorin, etc. that present varying ratios of protein to glycan.

### Example 9

### Further Characterization

To establish standards for confirming batch consistency in the MSA reagents, lectins will be coupled to beads using standard protocols. The amount of unbound lectin will be measured by UV absorption. Additionally, the standardized glycans (Table 9) will be titrated against the beads to determine if the maximum loading capacity is within an acceptable range.

To quantify the ability of MSA reagents employed in a multiplexed analysis to reproduce the relative levels of stoichiometric mixtures of representative glycan structures, stoichiometric mixtures of the standardized glycans will be used to establish normalized fluorescence intensities. The maximum fluorescence intensity for each batch of glycoprofiling reagent beads will be determined by titrating with standardized glycans, such as those presented in Table 9. The glycan concentration at saturation will be employed to determine mixture stoichiometry. Based on this analysis the precision with which the Glycoprofiling MSA can reproduce the known glycan ratios will be determined.

For further testing, a Glycoprofiling MSA with specificity for at least 6 representative glycan structures associated with eukaryotic glycosylation, based on at least 12 glycan-binding reagents, will be extended by including reagents with additional and redundant specificities: such as the cholera toxin B subunit (CTB), as well as lectins from Canavalia ensiformis (ConA), Lens culinaris (LCH), Galanthus nivalis (GNA), peanut (PNA), Erythrina cristagalli (ECA), Phaseolus vulgaris (PHA), wheat germ (WGA), Sambucus nigra I (SNA-I), Maackia amurensis II (MAL II), Aleuria aurantia (AAL), Ulex europaeus (UEA), Polyporus squamosus (PSL), Griffonia simplicifolia II (GS II). Any of the wide variety of commercially available lectins and carbohydrate-binding antibodies, including, but not limited to, any of those described herein, may be used. In addition, engineered carbohydrate-binding proteins may be employed.

Reagents for incorporation into a glycoprofiling MSA will be selected that have specificity for at least six of the following glycosylation sequences: Neu5Acα2-6Gal, Neu5Acα2-3Galβ, terminal Galβ1-4GlcNAc, terminal GlcNAcβ, bisecting GlcNAcβ, terminal Manα, and terminal Fucα. In addition, wherever possible, selected reagents will have had their glycan binding patterns determined from specificity data generated by screening against over 300 glycans as reported by the Consortium for Functional Glycomics (CFG) (see the world wide web at functionalglycomics.org).

In addition to demonstrating the capabilities of the Glycoprofiling MSA method described herein to distinguish between standardized samples of glycans relevant to protein glycosylation patterns and to characterize glycosylation profiles with standardized samples of glycoproteins, the Glycoprofiling MSA method described herein will be used to monitor glycosylation profiles during bioprocessing. The glycosylation pattern of glycoproteins isolated at various time points during glycoprotein expression will be determined. Glycosylation profiles for purified glycoprotein samples typical of those in biopharmaceutical or research laboratory environments will be determined. The accuracy of the data obtained will be independently confirmed using complementary analytical methods. The performance of the glycoprofiling MSA products, kits, and method described herein will be evaluated in commercially available flow cytometer systems from at least three established vendors.

### Example 10

### Lectin MSA Reagents

As additional MSA glycoprofiling reagents, lectins, including, but are not limited to, any of those listed below, will be coupled to beads using standard protocols.

| Lectin | Specificity |
|---|---|
| Concanavalin A from Canavalia | α-Man; α-Glc (to a lesser extent); α-GlcNAc; α- |
| ensiformis (Jack bean) (Con A) | linked mannose; and succinyl Con A: α-Man, α-Glc |
| Datura stramonium (DSA) | β-GlcNAc,4GlcNAc oligomers; LacNAc; (β-1,4) |
| | linked N-acetylglucosamine oligomers, preferring |
| | chitobiose or chitotriose over a single N- |
| | acetylglucosamine residue, N-acetyllactosamine and |
| | oligomers containing repeating N-acetyllactosamine |
| | sequences |
| Dolichos biflorus agglutinin (DBA) | Terminal α-GalNAc; Blood Group A |
| Garden pea, Pisum sativum agglutinin | α-Man; α-Glc; α-GlcNAc; Biantennary and |
| (PSA) | triantennary oligosaccharides with core fucose; |
| | Fucα1,6-GlcNAc important in recognition; α-linked |
| | mannose-containing oligosaccharides, with an N- |
| | acetylchitobiose-linked α-fucose residue included in |
| | the receptor sequence |
| Jacalin, Artocarpus integrifolia | α-Gal; α-GalNAc; Core β1,3GalNAc (T Antigen); |
| | α-Gal-OMe; O-glycosidically linked |
| | oligosaccharides, preferring the structure galactosyl |
| | (β-1,3) N-acetylgalactosamine. will bind this |
| | structure even in a mono- or disialylated form |
| Lentil, Lens culinaris agglutinin (LCA | α-Man; α-Glc; α-GlcNAc; fucose linked to |
| or LcH) | chitobiose core of N-linked oligosaccharide |
| | enhances binding; α-linked mannose residues, by |
| | recognizing additional sugars as part of the receptor |
| | structure LCA has a narrower specificity than Con |
| | A. For example, an α-linked fucose residue attached |
| | to the N-acetylchitobiose portion of the core |
| | oligosaccharide markedly enhances affinity |
| Lotus, Lotus tetragonolobus lectin, | α-Fuc; alpha-linked L-fucose containing |
| winged or asparagus pea (LTL) | oligosaccharides; α-L-Fuc |
| Maackia amurensis (MAA) Lectin I | Neu5Acα2,3Galβ1,4GlcNAc; Sialic Acid; α-Neu |
| (MAL I) and Lectin II (MAL II) | NAc (2→3)Gal; MAL I: galactosyl (β-1,4) N- |
| | acetylglucosamine structures. Maackia amurensis |
| | lectin I seems to tolerate substitution of N- |
| | acetyllactosamine with sialic acid at the 3 position |
| | of galactose however, MAL I does not appear to |
| | bind this structure when substitution with sialic acid |
| | is on the 6 position of galactose; MAL II: appears to |
| | bind sialic acid in an (α-2,3) linkage |
| Peanut, Arachis hypogaea (PNA) | β-Gal; β-Gal(1→3)GalNAc; Galβ1,3GalNAc (T |
| | antigen); Galβ1,3GalNAcα-O—Me (T antigen, α- |
| | Methyl Glycoside); galactosyl (β-1,3) N- |
| | acetylgalactosamine |
| Red kidney bean, Phaseolus vulgaris | α-GalNAc; β-GalNAc; Complex biantennary |
| Erythroagglutinin (PHA-E) | oligosaccharides with outer galactose and bisecting |
| | GlcNAc |
| Red kidney bean, Phaseolus vulgaris | α-GalNAc; β-GalNAc; Triantennary and |
| Leucoagglutinin (PHA-L) | tetraantennary complex oligosaccharides |
| Potato, Solanum tuberosum (STA) | β-GlcNAc; GlcNAcβ1,4-R; oligomers of N- |
| | acetylglucosamine and some bacterial cell wall |
| | oligosaccharides containing N-acetylglucosamine |
| | and N-acetylmuramic acid |
| Sambucus nigra (SNA or EBL) | Neu5Acα2,6Gal; Neu5Acα2,6GalNAc; β-Gal; |
| | Sialic Acid; α-NeuNAc(2→6) Gal/GalNAc; sialic |
| | acid attached to terminal galactose in (α-2,6), and to |
| | a lesser degree, (α-2,3), linkage |
| Slug, Limax flavus (LFA) | Neu5Ac; NeuGc; Sialic Acid |
| Soybean, Glycine soja or Glycine max | α- or β-GalNAc; GalNAc; Gal (to a lesser extent); |
| (SBA) | oligosaccharide structures with terminal α- or β- |
| | linked N-acetylgalactosamine, and to a lesser extent, |
| | galactose residues |
| Tomato, Lycopersicon esculentum | GlcNAcβ1,4GlcNAc oligomers; β-GlcNAc; N- |
| (LEA or LEL or TL) | acetylglucosamine oligomers, tomato lectin prefers |
| | trimers and tetramers of this sugar |
| Tritrichomonas mobilensis | Neu5Ac; NeuGc (to a lesser extent) |
| Ulex europaeus I (UEA I) | Blood Group H oligosaccharides, |
| | Fucα1,2Galβ1,4GlcNAc; α-Fucose; α-linked fucose |
| | residues; α-L-Fuc |
| Vicia villosa (VVA or VVL) | Tn antigen; GalNAcα1-O-Serine; mannose; α-Man? |
| | β-Man?; α-GalNAc; alpha- or beta-linked terminal |
| | N-acetylgalactosamine, especially a single alpha N- |
| | acetylgalactosamine residue linked to serine or |
| | threonine in a polypeptide (the “Tn antigen”) |
| Wheat Germ agglutinin, Triticum | (GlcNAc)2; (GlcNAc)3; Neu5Ac; β-GlcNAc; Sialic |
| vulgaris (WGA) | Acid; NeuNAc; N-acetylglucosamine, with |
| | preferential binding to dimers and trimers of this |
| | sugar. WGA can bind oligosaccharides containing |
| | terminal N-acetylglucosamine or chitobiose; |
| | succinylated WGA does not bind to sialic acid |
| | residues, unlike the native form, but retains its |
| | specificity toward N-acetylglucosamine |
| Wisteria floribunda (WFA or WFL) | Terminal GalNAcβ1,4->> Terminal GalNAcα1,3- |
| | or Terminal GalNAcβ1,3-; α-GalNAc; β-GalNAc; |
| | GalNAc; carbohydrate structures terminating in N- |
| | acetylgalactosamine linked alpha or beta to the 3 or |
| | 6 position of galactose |
| Galanthus nivalis (GNA or GNL) | α-Man; non-reduc. D-Man; (α-1,3) mannose |
| | residues; will not bind alpha linked glucose; |
| Vicia faba (VFA) | α-Man; α-Glc; α-GlcNAc; |
| Narcissus pseudonarcissus (NPA or | α-Man? β-Man?; alpha linked mannose, preferring |
| NPL) | polymannose structures containing (α-1,6) linkages |
| Chick pea, Cicer arietinum (CPA) | α-Man? β-Man?; Fetuin |
| Griffonia (Bandeiraea) simplicifolia II | α-GlcNAc; β-GlcNAc; alpha- or beta-linked N- |
| (GS II or GSL II) | acetylglucosamine residues, increasing the number |
| | of N-acetylglucosamine residues beyond two does |
| | not improve affinity; recognize exclusively alpha- |
| | or beta-linked N-acetylglucosamine residues on the |
| | nonreducing terminal of oligosaccharides |
| Laburnum alpinum (LAA) | β-GlcNAc |
| Oryza sativa (OSA) | β-GlcNAc |
| Ulex europaeus II (UEA II) | β-GlcNAc |
| Urtica dioica (UDA) | β-GlcNAc |
| Vigna radiate (VRA) | α-Gal |
| Psophocarpus tetragonolobus, Winged | α-Gal? β-Gal; GalNAc, Gal; PTL I: alpha linked |
| bean (PTA) Lectin I (PTL I) or Lectin | galactosamine; PTL II: binds preferentially to |
| II (PTL II) | galactosides, with N-acetylgalactosamine being the |
| | most inhibitory monosaccharide. However, in |
| | contrast to PTL I, this lectin prefers the beta |
| | anomeric configuration. PTL II shows a high |
| | affinity toward blood group H structures and the T- |
| | antigen |
| Garden snail, Helix aspersa (HAA) | α-GlcNAc; α-GalNAc; GalNAc |
| Griffonia (Bandeiraea) simplicifolia I | α-Gal; α-GalNAc; mixture of the five isolectins: A- |
| (GS I or BS I or GSL I) | rich lectin specific for α-N-acetylgalactosamine |
| | residues, while the B-rich lectin specific for α- |
| | galactose residues; Isolectin B4 (GS I-B4 or BS I- |
| | B4): α-Gal; Isolectin A4 (GS I-A4 or Bs I-A4): α- |
| | GalNAc |
| Edible snail, Helix pomatia (HPA) | α-GalNAc; GalNAc |
| Maclura pomifera (MPA or MPL) | α-Gal; α-GalNAc; alpha linked N- |
| | acetylgalactosamine structures |
| Colchicum autumnale (CA) | α-Gal? β-Gal? α-GalNAc? B-GalNAc? |
| mistletoe, Viscum album (VAA) | β-Gal |
| Allomyrina dochotoma (Allo A) | β-Gal |
| mushroom, Agaricus bisporus (ABA) | β-Gal; β-Gal(1→3)GalNac |
| Abrus precatorius (APA) | β-Gal |
| Cytisus scoparius (CSA) | β-Gal |
| Trichosanthes kirilowii (TKA) | β-Gal |
| castor bean, Ricinus communis I | β-Gal; oligosaccharides ending in galactose but may |
| (RCA I); RCA₁₂₀ | also interact with N-acetylgalactosamine |
| castor bean, Ricinus communis II | β-Gal; β-GalNAc; galactose or N- |
| (RCA II); RCA₆₀, Ricin, A chain | acetylgalactosamine residues |
| coral tree, Erythrina cristagalli (ECA | α-Gal; β-Gal; α-GalNAc; β-GalNAc; β- |
| or ECL) | Gal(1→4)GlcNAc; galactose residues and appears |
| | to have the highest binding activity toward |
| | galactosyl (β-1,4) N-acetylglucosamine |
| Siberian pea tree, Caragana | α-Gal; β-Gal; α-GalNAc; β-GalNAc; GalNAc |
| arborescens (CAA) | |
| Phaseolus lunatus (LBA) | α-GalNAc |
| Bauhinia purpurea (BPA or BPL) | α-GalNAc; β-GalNAc; galactosyl (β-1,3) N- |
| | acetylgalactosamine structures but oligosaccharides |
| | with a terminal alpha linked N-acetylgalactosamine |
| | can also bind |
| Aegopodium podagraria (APP) | α-GalNAc; β-GalNAc |
| Bryonia dioica (BDA) | α-GalNAc; β-GalNAc |
| Tulip lectin (TL) | α-GalNAc; β-GalNAc |
| Sophora japonica (SJA) | β-GalNAc; carbohydrate structures terminating in |
| | N-acetylgalactosamine and galactose residues, with |
| | preferential binding to β anomers |
| Anguilla Anguilla (AAA) | α-Fucose |
| horseshoe crab, Limulus polyphemus | Sialic Acid; NeuNAc; (Neu5Ac)•2,6- |
| (LPA) | GalNAc group |
| Homarus americanus (HMA) | α-GalNAc; α-Fucose; Sialic Acid |
| Cancer antennarius (CCA) | Sialic Acid |
| Vicia graminea (VGA) | |
| Euonymus europaeus (EEL) | type 1 or type 2 chain blood group B structures but |
| | will bind other oligosaccharides containing |
| | galactosyl (α-1,3) galactose; type 1 chain blood |
| | group H structures; |
| Robinia pseudoaccacia (RPA) | |
| Salvia horminum (SHA) | |
| Salvia sclarea (SSA) | |
| Perseau Americana (PAA) | |
| Mangifera indica (MIA) | |
| Iberis amara (IAA) | |
| Sarothamnus scoparius (SRA) | |
| Trifolium repens (RTA) | |
| Green marine algae, Codium fragile | GalNAc |
| Human Galectin-1 (Gal-1) | β-Gal |
| Human Galectin-3 (Gal-3) | β-Gal |
| Human Galectin-3C | β-Gal |
| red kidney bean, Phaseolus Vulgaris | |
| Agglutinin (PHA-E + L) | |
| red kidney bean, Phaseolus vulgaris | |
| Phytohemagglutinin (PHA-P) | |
| red kidney bean, Phaseolus vulgaris | |
| Mucoprotein (PHA-M) | |
| Pokeweed, Phytolacca americana | (GlcNAc)3 |
| (PWM) | |
| Pseudomonas aeruginosa (PA-I) | Gal |
| Rat Galectin-8 (Gal-8) | β-Gal |
| Aleuria Aurantia Lectin (AAL) | fucose linked (α-1,6) to N-acetylglucosamine or to |
| | fucose linked (α-1,3) to N-acetyllactosamine related |
| | structures |
| Amaranthus Caudatus Lectin (ACL or | galactosyl (β-1,3) N-acetylgalactosamine structure |
| ACA) | (“T-antigen”), tolerate sialic acid substitution at the |
| | 3 position of galactose in the “T” antigen |
| Hippeastrum Hybrid Lectin (HHL or | only alpha mannose residues, not alpha glucosyl |
| AL) | structures. an extended binding site for |
| | polymannose structures, not requiring mannose to |
| | be at the non-reducing terminus. binds both (α-1,3) |
| | and (α-1,6) linked mannose structures, as well as |
| | some yeast galactomannans |
| Ricin B Chain | |

### Example 11

### Anticarbohydrate Antibody MSA Reagents

As additional MSA glycoprofiling reagents, anti-carbohydrate antibodies will be coupled to beads using standard protocols. For example, antibody-bearing beads may be prepared by incubating 20 μL of carboxylated microspheres (5−7.2×10⁷/mL) with 20 μL antibody (1 mg/mL) in PBS for 15 min. Two microliters of NHS (50 mg/mL) and 2 μL of EDAC (50 mg/mL) were added, and the beads incubated for one hour at 4° C. Microspheres are washed twice with PBS plus 0.02% Tween20 (PBST) and resuspended to a concentration of 5×10⁷/mL.

Anti-carbohydrate antibodies include, but are not limited to, any of the following. Blood Group H n/ab antigen (86-M) Antibody (Abcam No. ab24776; Santa Cruz Biotechnology No. sc-52372); Blood Group A antigen (9A) Antibody (Abeam No. ab20131; GeneTx No. GTX40131; Santa Cruz Biotechnology No. sc-53180); Blood Group A antigen (HE-193) Antibody (Abeam No. ab2521; GeneTx No. GTX22521; Santa Cruz Biotechnology No. sc-59460); Blood Group A antigen (HE-195) Antibody (Abeam No. ab2522; GeneTx No. GTX22522); Blood Group A antigen (T36) Antibody (Abeam No. ab3353; GeneTx No. GTX23353); Blood Group A, B and H antigens (RE-10) Antibody (Abeam No. ab2523; GeneTx No. GTX22523; Santa Cruz Biotechnology No. sc-59459); Blood Group A1B antigen (HE-24) Antibody (Abeam No. ab2525; GeneTx No. GTX22525); Blood Group AB antigen (Z5H-2/Z2A) Antibody (Abeam No. ab24223); Blood Group antigen Precursor (K21) Antibody (Abeam No. ab3352; GeneTx No. GTX23352); Blood Group B antigen (CLCP-19B) Antibody (Abeam No. ab3354); Blood Group B antigen (HEB-29) Antibody (Abeam No. ab2524; GeneTx No. GTX22524; Santa Cruz Biotechnology No. sc-59463); Blood Group B antigen (Z5H-2) Antibody (Abeam No. ab24224); Blood Group H ab antigen (87-N) Antibody (Abeam No. ab24222; Santa Cruz Biotechnology No. sc-52369); Blood Group A1, A2 antigen (87-G) Antibody (Santa Cruz Biotechnology No. sc-52368); Blood Group H1 (O) antigen (17-206) Antibody (Abeam No. ab3355; GeneTx No. GTX23355); Blood Group H1+Blood Group H2 (0.BG.5) Antibody (Abeam No. ab31754); Blood Group H2 (0.BG.6) Antibody (Santa Cruz Biotechnology No. sc-59466); Blood Group Kell antigen (0.BG.7) Antibody (Abeam No. ab31771); Blood Group H2 antigen (BRIC231) Antibody (Abeam No. ab33404); Blood Group Kell Antigen (BRIC 203) Antibody (Abeam No. ab11463); Sialyl Tn (BRIC111) Antibody (Abeam No. ab24005); Blood Group Wrb (BRIC14) Antibody (Santa Cruz Biotechnology No. sc-59476); Blood Group H2 (BRIC231) Antibody (Santa Cruz Biotechnology No. sc-59467); Blood Group Kell antigen (MM0435-12X3) Antibody (Abeam No. ab90456); CD239 (MM0107-1M39) Antibody (Abeam No. ab89142); Blood Group Kell Antigen (RM0118-7L32) Antibody (Abeam No. ab86793); Blood Group Lewis (2Q398) Antibody (Abeam No. ab68390); Blood Group Lewis a (7LE) Antibody (Abeam No. ab3967; GeneTx No. GTX23967; Santa Cruz Biotechnology No. sc-51512); Blood Group Lewis a (PR 5C5) Antibody (Abeam No. ab70473); Blood Group Lewis a (PR 4D2) Antibody (Santa Cruz Biotechnology No. sc-53181); Blood Group Lewis a (SPM522) Antibody (Abeam No. ab64099; Santa Cruz Biotechnology No. sc-135725); CA19-9 (SPM110) Antibody (Abeam No. ab15146; Santa Cruz Biotechnology No. sc-56506); Blood Group Lewis a (SPM279) Antibody (Santa Cruz Biotechnology No. sc-52988); Blood Group Lewis a (T174) Antibody (Abeam No. ab3356; GeneTx No. GTX23356; Santa Cruz Biotechnology No. sc-59469); Blood Group Lewis b (2-25LE) Antibody (Abeam No. ab3968; GeneTx No. GTX23968; Santa Cruz Biotechnology No. sc-51513); Blood Group Lewis b antibody (LWB01; same as 2-25LE) Antibody (Abeam No. ab44959; GeneTx No. GTX72378); Blood Group Lewis b (T218) Antibody (Abeam No. ab3357; Santa Cruz Biotechnology No. sc-59470); Blood Group Lewis x (4C9) Antibody (Abeam No. ab52321; Santa Cruz Biotechnology No. sc-69905); Blood Group Lewis x (P12) Antibody (Abeam No. ab3358; GeneTx No. GTX23358; Santa Cruz Biotechnology No. sc-59471); Blood Group Lewis y (A70-C/C8) Antibody (Abeam No. ab23911; Santa Cruz Biotechnology No. sc-59472); Blood Group Lewis y (F3) antibody (Abeam No. ab3359; GeneTx No. GTX23359); Blood Group N antigen (DRF-8) Antibody (Abeam No. ab24217; Santa Cruz Biotechnology No. sc-52374); Blood Group Tn antigen (Tn 218) Antibody (Abeam No. ab76752); Blood Group Wrb (E6) Antibody (Abeam No. ab50293; Santa Cruz Biotechnology No. sc-81763); Blood group H inhibitor (97-I) Antibody (Abeam No. ab24213); CA19-9 (0.N.36) Antibody (Abeam No. ab33181); CA19-9 (121SLE) Antibody (Abeam No. ab3982); Sialyl Lewis a (121SLE) Antibody (Santa Cruz Biotechnology No. sc-51696); CA19-9 (BC/121SLE) Antibody (Abeam No. ab2707); CA19-9 (192) Antibody (Abeam No. ab25802; Santa Cruz Biotechnology No. sc-59480); CA19-9 (241) Antibody (Santa Cruz Biotechnology No. sc-59481); CA19-9 (8.F.26) Antibody (Santa Cruz Biotechnology No. sc-73411); CD77 (38-13) Antibody (Abeam No. ab19795); Blood Group M antigen (GH-9) Antibody (Abeam No. ab24215; Santa Cruz Biotechnology No. sc-52373); Sialyl Tn (STn 219) Antibody (Abeam No. ab76754); CD15 (28) Antibody (Abeam No. ab20137); CD15 (DU-HL60-3) Antibody (Abeam No. ab13453); CD15 murine monoclonal (MC480) (Abeam No. ab16285); CD15 (MY-1) Antibody (Abeam No. ab754); Blood Group B antigen (Z5H-2) Antibody (GeneTx No. GTX44224; Santa Cruz Biotechnology No. sc-69952); Blood Group AB antigen (Z5H-2/Z2A) Antibody (GeneTx No. GTX44223; Santa Cruz Biotechnology No. sc-52370); Blood Group Lewis a/b (HEA164) Antibody (Santa Cruz Biotechnology No. sc-73368); Blood Group Lewis a (B369) Antibody (Santa Cruz Biotechnology No. sc-59468); Blood Group A antigen (Z2A) Antibody (Santa Cruz Biotechnology No. sc-69951); Blood Group A antigen (B45.1) Antibody (Santa Cruz Biotechnology No. sc-59457); Blood Group A antigen (B480) Antibody (Santa Cruz Biotechnology No. sc-59458); Blood Group A1, A2, A3 antigen (1V015) Antibody (Santa Cruz Biotechnology No. sc-70427); Blood Group A1, A2, A3 antigen (Z2B-1) Antibody (Santa Cruz Biotechnology No. sc-52367); Blood Group B antigen (89-F) Antibody (Santa Cruz Biotechnology No. sc-52371); Blood Group H2 (A46-B/B10) Antibody (Santa Cruz Biotechnology No. sc-65680); Blood Group H2 (A51-B/A6) Antibody (Santa Cruz Biotechnology No. sc-65682); Blood Group M antigen (1.B.710) Antibody (Santa Cruz Biotechnology No. sc-70428); Forssman Antigen (M1/87) Antibody (Santa Cruz Biotechnology No. sc-23939); Forssman Antigen (M1/87.27.7.HLK) Antibody (Santa Cruz Biotechnology No. sc-81724); CD15s (CHO131) Antibody (Santa Cruz Biotechnology No. sc-32243); and CD15s (5F18) Antibody (Santa Cruz Biotechnology No. sc-70545).

### Example 12

### Disease Targets

The glycoprofiling MSA technology described herein may be applied to the diagnosis of a variety of diseases, including, but not limited to, any of those described below.

| Target | Disease | Current Reagent |
|---|---|---|
| beta(1,6)-branching | Breast Carcinoma: During the oncogenesis of breast | PHA-L |
| of polylactosamine | carcinoma, the glycosyltransferase known as N- | (Kaneda et al., |
| chains | acetylglucosaminyltransferase Va (GnT-Va) | 2002, J Biol |
| GlcNAcb(1-6)Gal | transcript levels and activity are increased due to | Chem; 277: 16928-16935) |
| mostly endo | activated oncogenic signaling pathways. Elevated | |
| | GnT-V levels leads to increased β(1,6)-branched N- | |
| | linked glycan structures on glycoproteins | |
| | (Abbott et al., 2008, J Proteome Res; 7(4): 1470-80) | |
| Polylactosamine | Cold Agglutinin Disease: Auto-antibodies react with | DSL, DSA |
| basis for beta(1,6) | the “i” antigen, can be triggered by infection with M. pneumonia. | |
| branching of “i” | | |
| Blood Group | | |
| Antigen | | |
| [Galb(1- | | |
| 4)GlcNAcb(1-3)]n | | |
| Polylactosamine | Cold Agglutinin Disease | DSL, DSA |
| basis for beta(1,6) | | |
| branching of “i” | | |
| Blood Group | | |
| Antigen | | |
| [GlcNAcb(1- | | |
| 3)Galb(1-4)]n | | |
| Bisecting GlcNAc | Related to antibody effector function, autoimmune | PHA-E |
| GlcNAcb(1-4)Man | disease, antigen binding | (Kaneda et al., |
| | (Arnold et al., 2007, Ann Rev Immunol; 25: 21-50) | 2002, J Biol |
| | | Chem; 277: 16928-16935) |
| Bisecting GlcNAc | Normal liver cells and primary adult hepatocytes are | PHA-E |
| GlcNAcb(1-4)Man | characterized by a very low level of GlcNAc- | (Kaneda et al., |
| | transferase-III activity, whereas human hepatoma | 2002, J Biol |
| | cells exhibited high activities | Chem; 277: 16928-16935) |
| | (Song et al., 2001, Cancer Invest; 19(8): 799-807) | |
| core alpha-1,6- | Hepatocellular carcinoma: woodchucks diagnosed | Array of |
| linked fucose | with HCC have dramatically higher levels of serum- | lectins from Lens |
| Fuca(1-6)GlcNAcb | associated core α-1,6-linked fucose, as compared with | culinaris, Pisum |
| | woodchucks without a diagnosis of HCC | sativum, |
| | (Block et al., 2005, Proc Natl Acad Sci USA; | and Vicia faba. |
| | 102: 779-84) | |
| core alpha-1,6- | Related to antibody effector function, autoimmune | Array of |
| linked fucose | disease, antigen binding | lectins from Lens |
| Fuca(1-6)GlcNAcb | (Arnold et al., 2007, Ann Rev Immunol; 25: 21-50) | culinaris, Pisum |
| | | sativum, |
| | | and Vicia faba. |
| Outer arm | Pancreatic Cancer: Forty-four oligosaccharides were | ConA, UEA-I |
| fucosylation | found to be distinct in the pancreatic cancer serum. | (ConA lectin |
| Fuca(1-2)Gal (A, | Increased branching of N-linked oligosaccharides and | affinity |
| B, H, Le^{y}, Le^{b} | increased fucosylation and sialylation were observed | chromatography, |
| antigen) | in samples from patients with pancreatic cancer | the recovery for |
| | (Zhao et al., 2007, J Proteome Res; 6(3): 1126-1138) | N-linked glycan |
| | | structures with a |
| | | mannose core |
| | | such as complex |
| | | type glycans is |
| | | lower than the |
| | | high mannose |
| | | glycan structure |
| | | proteins.) |
| Fuca(1-2)Galb | Prostate and Colon Cancer: A characteristic feature | PNA |
| | of tumor progression in distal colon and rectum is the | |
| | expression of the blood group determinants Le^{b}, H- | |
| | type 2 and Le^{y}, as well as the glycolipid Globo H, | |
| | which contain the motif Fuca(1-2)Galβ-R | |
| | (Chandrasekaran et al., 2002, Glycobiol; 12: 153-162) | |
| Outer arm | (Chandrasekaran et al., 2002, Glycobiol; 12: 153-162) | Blood Group |
| fucosylation | | Lewis x antibody |
| Fuca(1-3)GlcNAc | | [P12] |
| (Le^{x} antigen) | | |
| Antigen Le^{y} | Aberrant glycosylation has been associated with the | MAb AH6 |
| Fuca(1-2)Galb(1- | malignant phenotype in various tissues, and certain | MAb B3 |
| 4)[Fuca(1- | alterations in oligosaccharides have been associated | Antibody AH6, |
| 3)]GlcNAcb1-R | with the metastatic process and poor patient survival | IgM and TKH2, |
| | in several carcinomas. These include increase in | IgG. |
| | Lewis y (Le^{y}), Sialyl Lewis x (Sle^{x}), Sialyl Tn (STn), | |
| | and Tn expression (Davidson et al., 2000, Hum | |
| | Pathol; 31: 1081-1087). | |
| Le^{x} epitope | Cancer Metastasis: N-linked glycosylation from a | All by MS |
| Galb(1-4)[Fuca(1- | nonmetastatic brain tumor cell line and two different | |
| 3)]GlcNAcb(1- | metastatic brain tumor cells were compared (Prien et | |
| 3)Gal | al., 2008, Glycobiol; 18: 353-366) | |
| Outer arm | (Prien et al., 2008, Glycobiol; 18: 353-366) | Blood Group |
| fucosylation | | Lewis a antibody |
| Fuca(1-4)GlcNAc | | [SPM522] |
| (Le^{a}, Le^{b} antigen) | | |
| terminal Neu5Ac | Related to antibody effector function, autoimmune | SNA |
| Neu5Aca(2-6)Gal | disease, antigen binding (Arnold et al., 2007, Ann Rev | |
| | Immunol; 25: 21-50) | |
| Terminal Neu5Ac | Pancreatic Cancer: Forty-four oligosaccharides were | ConA |
| Neu5Aca(2-6)Gal | found to be distinct in the pancreatic cancer serum. | (lectin affinity |
| | Increased branching of N-linked oligosaccharides and | chromatography, |
| | increased fucosylation and sialylation observed in | the recovery for |
| | samples from patients with pancreatic cancer | N-linked glycan |
| | (Zhao et al., 2007, J Proteome Res; 6: 1126-1138) | structures with a |
| | | mannose core |
| | | such as complex |
| | | type glycans is |
| | | lower than the |
| | | high mannose |
| | | glycan structure |
| | | proteins.) |
| Terminal Neu5Ac | (Zhao et al., 2007, J Proteome Res; 6: 1126-1138) | MAA |
| Neu5Aca(2-3)Gal | | |
| Sialyl-Lewis X | (Zhao et al., 2007, J Proteome Res; 6: 1126-1138) | MAb 2H5 |
| Neu5Aca(2- | | Antibody 2H5, |
| 3)Galb(1-4)[Fuca1- | | IgM (PharMingen, |
| 3]GlcNAc-R | | Becton Dickinson, |
| | | San Jose, CA) |
| Neu5Aca(2-3)Gal | Influenza receptor (Horimoto and Kawaoka, 2005, | MAA |
| | Nat Rev Microbiol; 3: 591-600) | |
| Neu5Aca(2-6)Gal | Influenza receptor (Horimoto and Kawaoka, 2005, | LPA, SNA |
| | Nat Rev Microbiol; 3: 591-600) | |
| terminal Neu5Ac | IgA nephropathy: the IgA glycoform from IgAN | MAA |
| Neu5Aca(2-3)Gal | patients highly expressing GalNAc or Neu5Ac- | |
| | 2,6,GalNAc significantly depressed the Mesangial | |
| | Cell proliferation rate (Coppo and Amore, 2004, | |
| | Kidney International 65: 1544-1547) | |
| terminal Neu5Ac | IgA nephropathy: N-linked (Coppo and Amore, 2004, | SNA |
| Neu5Aca(2- | Kidney International; 65: 1544-1547) | |
| 6)GalNAc | | |
| Sialyl-Tn | Common feature in mucins associated with | MAb TKH2 |
| Neu5Aca(2- | carcinomas | |
| 6)GalNAca1-O- | | |
| Ser/Thr | | |
| Found on MUC1 | | |
| terminal Gal | IgA nephropathy: N-linked (Coppo and Amore, 2004, | WGA, Jacalin |
| Galb1-3GalNAc | Kidney International; 65: 1544-1547) | |
| TF-antigen | Associated with carcinomas (colon cancer): The | PNA, ABA |
| Galb(1-3)GalNAc | glycosylation changes include increased expression of | |
| found on MUC1 | onco-fetal carbohydrates, such as the galactose- | |
| | terminated Thomsen-Friedenreich antigen | |
| | (Galβ1,3GalNAcα-), increased sialylation of terminal | |
| | structures and reduced sulphation | |
| terminal | Related to antibody effector function, autoimmune | MAL I |
| galactosylation | disease, antigen binding (Arnold et al., 2007, Ann Rev | |
| Galb(1-4)GlcNAc | Immunol; 25: 21-50) | |
| Terminal GalNAc | IgA nephropathy: | SBA |
| GalNAc-OSer/Thr | (Amore and Coppo, 2000 Nephron 86: 255-259) | |
| Tn Antigen | Common feature in mucins associated with | MAb HB-Tn1 |
| GalNAca-O- | carcinomas | Antibody HB-Tn1, |
| Ser/Thr | | IgM (Dako, |
| Found on MUC1 | | Glostrup, |
| | | Denmark) VVL, |
| | | VVA |
| terminal GlcNAc | Related to antibody effector function, autoimmune | PHA-L |
| GlcNAcb(1-2)Man | disease, antigen binding (Arnold et al., 2007, Ann Rev | |
| | Immunol; 25: 21-50) | |
| terminal GlcNAc | (Arnold et al., 2007, Ann Rev Immunol; 25: 21-50) | STA |
| GlcNAcb(1-4)Man | | |
| terminal GlcNAc | (Arnold et al., 2007, Ann Rev Immunol; 25: 21-50) | |
| GlcNAcb(1-6)Man | | |
| N-glycolyl GM3 | This epitope is a molecular marker of certain tumor | MAb 14F7 |
| Neu5Gca(2-3)Gal | cells and not expressed in normal human tissues | |
| | (Arnold et al., 2007, Ann Rev Immunol; 25: 21-50) | |
| Terminal GlcNAc | Type II Diabetes: increased intracellular glycosylation | anti-O-GlcNAc |
| GlcNAcb-O- | of proteins via O-GlcNAc can induce insulin | antibody RL-2 and |
| Ser/Thr | resistance and that a rodent model with genetically | ERK-2, MAb |
| | elevated O-GlcNAc levels in muscle and fat displays | CTD110.6, |
| | hyperleptinemia (Lim et al., 2008, J Proteome Res; | |
| | 7(3): 1251-63; Comer et al., 2001, Anal Biochem; | |
| | 293: 169-177) | |
| tumor-associated | Tumor associated antigen: Antigen initially detected | The monoclonal |
| antigen 19-9 | in a human colorectal cell line | antibody CO 19-9 |
| Neu5Aca(2- | | is specific for the |
| 3)Galb(1- | | 19-9 |
| 3)[Fuca(1- | | antigen and does |
| 4)]GlcNAc | | not cross-react |
| Galb(1-3)GlcNAc | | with Le^{a} |
| Le^{a} blood group | Tumor associated antigen: | The monoclonal |
| antigen component, | (Bechtel et al., 1990, J Biol Chem; 265: 2028-2037) | antibody CO 19-9 |
| Galb(1-3)[Fuca(1- | | is specific for the |
| 4)]GlcNAc | | 19-9 |
| | | antigen and does |
| | | not cross-react |
| | | with Le^{a} |
| Globo H | Breast Cancer: The cell-surface glycosphingolipid | MBr1 (IgM, |
| Fuca(1-2)Galb(1- | Globo H is a member of a family of antigenic | Alexis |
| 3)GlcNAcb(1- | carbohydrates that are highly expressed on a range of | Biochemicals, |
| 3)Gala(1-4)Galb(1- | cancer cell lines, especially breast cancer cells | Lausen, |
| 4)Glcb | (Huang et al., 2006, Proc Natl Acad Sci USA; 103: 15-20; | Switzerland) and |
| | Wang et al., 2008, Proc Natl Acad Sci USA; | VK-9 (IgG). |
| | 105: 11661-11666) | |
| Globo H | Breast Cancer: | Glycoope antibody |
| Fuca(1-2)Galb(1- | (Huang et al., 2006, Proc Natl Acad Sci USA; 103: 15-20; | to Globo H A69- |
| 3)GlcNAcb(1- | Wang et al., 2008, Proc Natl Acad Sci USA; | A/E8 |
| 3)Gala(1-4)Galb(1- | 105: 11661-11666) | |
| 4)Glcb | | |
| Gb3 | The trisaccharide glycolipid Gb-3 is a receptor for | Anti-Gb3 Isotype |
| Gala(1-4)Glcb(1- | Shiga-like toxins and has recently been implicated in | IgM (1A4) |
| 4)Glcb-Cer | the entry of HIV-1 into cells (Werz et al., 2007, J Am | |
| | Chem Soc; 129: 2770-2771) | |
| Forssman Antigen | Various cancer tissues (Hakomori, 1984, Ann Rev | Forssman Antigen |
| GalNAca(1- | Immunol; 2: 103-26) | (M1/87) Antibody |
| 3)GalNAcb(1-3)- | | |
| Gala(1-4)Galb(1- | | |
| 4)Glcb(1- | | |
| Forssman Antigen | Various cancer tissues (Hakomori, 1984, Ann Rev | Forssman Antigen |
| GalNAca(1- | Immunol; 2: 103-26) | (M1/87) Antibody |
| 3)GalNAcb(1-3)- | | |
| Gala(1-4)Galb(1- | | |
| 4)Glcb(1- | | |
| GlcNAcb(1- | Common to all N-linked glycans and fundamental to | N/A |
| 4)GlcNAcb-N-Asn | many of the glycans in this table | |
| GlcNAcb(1- | Common to all N-linked glycans and fundamental to | N/A |
| 4)GlcNAcb-N-Asn | many of the glycans in this table | |
| GlcNAcb(1- | Hepatocellular carcinoma: woodchucks diagnosed | N/A |
| 4)[Fuca(1- | with HCC have dramatically higher levels of serum- | |
| 6)GlcNAcb-N-Asn | associated core α-1,6-linked fucose, as compared with | |
| | woodchucks without a diagnosis of HCC (Block et | |
| | al., 2005, Proc Natl Acad Sci USA; 102: 779-84) | |
| GlcNAcb(1- | Hepatocellular carcinoma: woodchucks diagnosed | N/A |
| 4)[Fuca(1- | with HCC have dramatically higher levels of serum- | |
| 6)GlcNAcb-N-Asn | associated core α-1,6-linked fucose, as compared with | |
| | woodchucks without a diagnosis of HCC (Block et | |
| | al., 2005, Proc Natl Acad Sci USA; 102: 779-84) | |
| [6S]GlcNS- | Glycosaminoglycans include heparin and are | N/A |
| [2S]IdoA | associated with viral adhesion (herpes) and some | |
| | cancers | |
| GlcNS-IdoA | Glycosaminoglycans include heparin and are | N/A |
| | associated with viral adhesion (herpes) and some | |
| | cancers | |
| GlcNS-GlcA | Glycosaminoglycans include heparin and are | N/A |
| | associated with viral adhesion (herpes) and some | |
| | cancers | |

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

1-93. (canceled)

94. An inactivated mutated carbohydrate-processing enzyme having enhanced affinity for its substrate compared to a corresponding wild-type carbohydrate-processing enzyme, wherein the mutated carbohydrate-processing enzyme comprises at least one inactivating mutation that eliminates catalytic activity of the enzyme and further independently comprises at least one affinity-enhancing mutation selected from
(a) a mutation of an amino acid residue located within 5 Å of the substrate in an enzyme-substrate complex, wherein the per-residue contribution of the amino acid residue to at least one of the total interaction energy (ΔE_{MM}) or the total binding free energy (ΔG_{Binding}) for amino acid residues in the enzyme-substrate complex is ≥−0.7 kcal/mol; or
(b) a mutation of an amino acid residue located more than 5 Å from the substrate in an enzyme-substrate complex, wherein the per-residue contribution of the amino acid residue to at least one of ΔE_{MM} or ΔG_{Binding} for amino acid residues in the enzyme-substrate complex is ≥0.0 kcal/mol.

95. The inactivated mutated carbohydrate-processing enzyme of claim 94, wherein the carbohydrate-processing enzyme is selected from the group consisting of a glycosidase, a glycosyltransferase, a polysaccharide lyase, a carbohydrate esterase, a sulfatase, a sulfotransferase, a ligase, and epimerase, and any other enzyme that acts on a carbohydrate substrate.

96. The inactivated mutated carbohydrate-processing enzyme of claim 94, wherein the carbohydrate processing enzyme is encoded by a gene from a prokaryotic organism.

97. The inactivated mutated carbohydrate-processing enzyme of claim 94, wherein the carbohydrate processing enzyme is encoded by a gene from a eukaryotic organism.

98. The inactivated mutated carbohydrate-processing enzyme of claim 94, wherein the carbohydrate processing enzyme is PNGase F, β-O-GlcNAcase, or neuraminidase.

99. The inactivated mutated carbohydrate-processing enzyme of claim 98, wherein the neuraminidase is encoded by a gene from Clostridium perfringens.

100. A composition comprising a mixture of two or more sets of individually addressable particles, each set of individually addressable particles comprising an external surface and having linked to said external surface a different carbohydrate binding molecule,
wherein at least one carbohydrate binding molecule comprises an inactivated mutated carbohydrate-processing enzyme of claim 94,
wherein each set of individually addressable particles is differently labeled with a detectable label.

101. The composition of claim 100, wherein the individually addressable particle comprises a bead or a nanoparticle.

102. The composition of claim 100, wherein the detectable label comprises an optically encoded fluorescent dye.

103. The composition of claim 100, comprising at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, or at least twenty different sets of individually addressable particles.

104. The composition of claim 100, comprising at least ten, at least twenty, at least thirty, at least forty, at least fifty, at least sixty, at least seventy, at least eighty, at least ninety, or at least one hundred different sets of individually addressable particles.

105. A kit comprising a composition of claim 100.

106. The kit of claim 105, further comprising a secondary detection reagent for detectably labelling an analyte.

107. A multiplex detection method for detecting a carbohydrate or a carbohydrate containing compound in a sample comprising:
contacting the sample with a composition of claim 100; and
detecting the binding of the carbohydrate or carbohydrate containing compound to one more individually addressable particles;
wherein the carbohydrate or carbohydrate containing compound bound to one more individually addressable particles remains in suspension.

108. The method of claim 107, wherein the individually addressable particle comprises a bead or a nanoparticle.

109. The method of claim 107, wherein the detectable label comprises an optically encoded fluorescent dye.

110. The method of claim 109, wherein the detection is by flow cytometry analysis.

111. The method of claim 107, wherein the sample is obtained during the production of a recombinant glycoprotein in the pharmaceutical or research industries.

112. The method of claim 107, monitoring glycosylation profiles during bioprocessing.

113. The method of claim 107, wherein the sample is an environmental or biological sample.
